(19) United States
(12) Patent Application Publication
(10) Pub. No.: US 2002/0155460 A1
(43) Pub. Date: Oct. 24, 2002

(54) Information rich libraries

(75) Inventors: Volker Schellenberger, Palo Alto, CA (US); Donald P. Naki, San Diego, CA (US); Thomas B. Morrison, Winchester, MA (US)

Correspondence Address: DAVID W. MAHER, McCutchen, Doyle, Brown & Enersen, LLP, Suite 1800, Three Embarcadero Center, San Francisco, CA, 94111, US

(73) Assignee: Genencor International Inc.

(21) Appl. No.: 09/975,139

(22) Filed: Oct. 10, 2001

Related U.S. Application Data
(60) Provisional application No. 60/239,476, filed on Oct. 10, 2000.

(51) Int. Cl. C12Q 1/68; C12P 19/34; C12N 9/04; C12N 9/16; C12N 9/24; C12N 9/50

(52) U.S. Cl. 435/6; 435/190; 435/196; 435/200; 435/219; 435/91.2

(57) ABSTRACT

Methods of creating libraries of biological polymers are provided. The construction of a library employs a probability matrix for a reference sequence, and a constraint vector for which is applied to the probability matrix to produce a substitution scheme. The substitution scheme is then used to generate a library comprising substitutions recommended by the substitution scheme. The library members, or host cells comprising and/or expressing them, can be screened for desired changes in a property of interest in the biological polymers in the library.

### CROSS-REFERENCES TO RELATED APPLICATIONS

This application claims the benefit of priority to U.S. Provisional Patent Application No. 60/239,476, filed Oct. 10, 2000.

### STATEMENT AS TO RIGHTS TO INVENTIONS MADE UNDER FEDERALLY SPONSORED RESEARCH AND DEVELOPMENT

Not Applicable.

### TECHNICAL FIELD

This invention relates to methods for producing information rich polynucleotide libraries and articles and compositions useful therein and produced thereby.

### BACKGROUND OF THE INVENTION

There is currently no effective way to systematically screen all possible permutations of a polymeric biological molecule such as a polynucleotide or protein for a property of interest where the molecule is of significant length. To test four nucleotides and 20 amino acids at each position in a polynucleotide or protein, respectively, rapidly leads to a geometric increase in the number of molecules to be tested such that available methods of synthesis, and even available volumes for testing, are quickly exceeded for even a small length of such a polymer. Furthermore, even if it were physically possible to screen all permutations of a sequence of a given length, the brute force nature of such an approach would result in a great deal of the effort expended being wasted in producing and characterizing molecules lacking the desired activity.

As a compromise, a number of different approaches have arisen to sample some of the diversity available in such polymeric biological molecules.

There are two well known methods for attempting to improve the function of a protein. In random mutagenesis, one introduces random mutations and then screens for mutants with a desirable change. Although introducing more mutations per gene increases the chances of finding genes with interesting functions, each mutation potentially leads to a non-functional protein (for instance by interfering with folding). Thus, if in creating a protein variant library, one increases the average number of mutations per gene, one then also increases the fraction of genes in the library that encode proteins which lack function.

Another method utilizes recombination between homologous coding sequences. The key advantage of recombination over random mutagenesis is that it introduces mutations known to function in a homologous protein. As a result, one generates libraries which have a relatively large diversity yet still contain a large fraction of functional mutants. In other words, recombination uses the information contained in homologous sequences to introduce diversity into a protein of interest. However, diversity in recombination is limited by the kind of information it can utilize (i.e., it uses only homologous sequences) and recombination is limited in the way it utilizes that information. For example, one has limited control over the selection of crossover points. In another example, recombination usually moves regions of a gene (10-1000 bp). It rarely moves an individual residue from one sequence into a homologous position in another sequence.

Systematic approaches to altering residues in biological polymers have been made. See, for example, the “SELEX” procedures described in Tuerk et al., Proc Natl Acad Sci U S A 1992 Aug 1, 89(15):6988-92, and the screening for aptamers as described in Bock et al., Nature 1992 Feb 6, 355(6360):564-6. Pools of degenerate molecules are tested for a desired activity and the molecules possessing the greatest level of such activity can be propagated and subjected to further rounds of mutagenesis and selection. Again, however, it is not possible to test all permutations of a sequence of any significant length, so such techniques are limited by a type of “founder effect” controlled by the number of different molecules actually present in the starting population.

Systematic approaches to mutating every position in a protein have also been performed. However, the diversity at any given position is typically limited to a single change. Furthermore, such changes are typically made and assayed individually, are not made in the form of a library, and therefore do not test for multiple mutations which may be required for any given mutation to exhibit its potential activity. In some cases, a number of multiple mutants have been made at different positions throughout a protein. However, these are again typically predefined, and do not result in the production of a library of different polymers.

Thus, there remains a need in the art for a mechanism to increase the diversity of polymeric biological molecules present in a library and to increase the proportion of members of that library having a desired activity.

### SUMMARY OF THE INVENTION

Methods to create information rich libraries, that is libraries that contain a high fraction of biological polymers having a desired activity are disclosed. The information used to create these libraries can include: multiple sequence alignments, substitution matrices, three dimensional structure, and prior knowledge about the structure and/or function of the reference sequence from which the library is to be produced of from a homologous sequence in a related molecule.

Generally speaking, the steps towards the manufacture of the libraries of this invention include generating a probability matrix, generating a constraint vector, designing a substitution scheme based on the probability matrix and constraint vector. The substitution scheme has utility as produced, and can be used to construct a library based thereon. The library can then be screened and the members of the library characterized. Data mining techniques can be employed to characterizing the functional clones. Optionally, the characterization data can be used as information in a subsequent iteration of the method to obtain a molecule with even more desirable properties.

Additionally, combinations of the methods described herein can be made with other techniques such as family shuffling and/or systematic scanning approaches can be performed in any order and for any number of iterations to produce the products described herein; such combinations are within the scope of the invention. Also provided are vectors containing polynucleotides produced by the disclosed methods, host cells comprising such vectors, proteins encoded by such polynucleotides, and libraries of members so generated.

### BRIEF DESCRIPTION OF THE DRAWINGS

FIG. 1 is a graphical representation of the relationship between a probability matrix and a constraint vector of this invention. After a probability matrix is generated, a constraint vector can be applied to the matrix to determine which amino acid substitutions will be selected to test for their effect on a desired functionality. In this graphical representation, the residues for which values calculated by the matrix rise above the constraint put on by the vector are candidates for the library.

FIG. 2 is an alignment of the sequence of ampC proteins from seven different organisms.

### DETAILED DESCRIPTION OF THE INVENTION

The prior art is replete with examples of techniques intended to improve the function of proteins and polynucleotides under defined conditions. One of the most well known examples utilizes crossover recombination or DNA shuffling. Diversity produced by DNA shuffling is limited to the parent sequences and random mutations.

The invention described herein can be used to introduce residues that are not contained in the parent reference sequence but that are still likely to preserve structure and function. Because a constraint of functionality is placed on the possible mutations, the fraction of inactivating mutations is minimized. This allows one to test higher mutation frequencies and increases the chance of finding useful double and triple mutations. For example, in a library of double mutants there is one chance per member to find interacting mutations. However, if one can generate a library of members of which 100% are active and contain 20 mutations per member then there are 190 possible pair-wise interactions between these mutations per member. In addition, the library will contain a large number of functional proteins with triple and higher mutations.

DNA shuffling recombines linear blocks of sequence. This places many amino acids into new environments at the same time because residues which are close in linear sequence are not necessarily close in three dimensional space. Conversely, computer shuffling techniques allow one to recombine residues which are close in three dimensional space. Thus, one can effect mutations in subdomains of the protein which are distant in linear sequence but close in structure, thus further increasing the chance to find interacting mutations.

Because DNA shuffling recombines linear blocks of sequence, beneficial mutations at one locus may be masked by detrimental mutations nearby. For illustration purposes only, Ballinger found that recruiting a furin residue into position 104 of Bacillus amyloliquefaciens subtilisin improved performance of the enzyme. However, recruiting a furin residue at position 107 abolished expression of the protein. Because these residues are very close, the chances of having a crossover event between them using DNA shuffling is remote and the resultant protein would not be active (if present at all) even though it contained a useful mutation. Ballinger, Biochemistry 34:13312 (1995); Ballinger, Biochemistry 35:13579 (1996).

Benefits of the invention described herein include greater control of the complexity of the library. For example, if a large number of functional proteins are desired, the constraint matrix can be constructed to include fewer substitutions likely to lead to non-functional proteins. If more diversity is desired, the constraint matrix can be constructed to provide a lower constraint on the probability matrix.

Because a library that has a higher percentage of mutated and functional proteins can be constructed, fewer members of the library are needed to achieve a suitable number of possible useful proteins. In a particular embodiment, one may characterize the sequence and function of most or all members of a population, including non-functional proteins. Thus, in addition to obtaining useful proteins with a minimal number of screening assays, one is able to obtain information as to which mutations are detrimental to a protein. This information can then be used in a new constraint matrix, for example for another iteration.

Knowledge-based approaches can incorporate information from mutation of the reference sequence into the substitution scheme. Such information can be derived from intentional mutagenesis, either sporadic or systematic, or can incorporate information from naturally occurring mutations. Systematic approaches can include saturation scans where each residue of a protein is individually changed to each of the other 19 genetically coded amino acids and the resulting single mutants screened for the desired property, as well as deletion mutagenesis scans where one or more residues are deleted from the protein, insertion mutagenesis scans where one or more residues are inserted in the protein, and alanine scanning mutagenesis where each residue of the protein is systematically replaced with an alanine. Although systematic approaches provide the most information, any mutation which provides information about the protein's ability to tolerate a mutation affecting the desired property can be used.

Before the present invention is described in detail, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a polynucleotide” includes a plurality of polynucleotides, reference to “a substrate” includes a plurality of such substrates, reference to “a variant” includes a plurality of capture probes, and the like.

Terms such as “connected,” “attached,” “linked,” and “conjugated” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Unless otherwise indicated, nucleic acids are written left to right in 5′ to 3′ orientation; amino acid sequences are written left to right in amino to carboxy orientation, respectively. The headings provided herein are not limitations on the invention, but exemplify the various aspects of the invention. Accordingly, the terms defined immediately below are more fully defined by reference to the specification as a whole.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

I. DEFINITIONS

The terms “polynucleotide,” “oligonucleotide,” “nucleic acid” and “nucleic acid molecule” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

Where the polynucleotides are to be used to express encoded proteins, nucleotides which can perform that function or which can be modified (e.g., reverse transcribed) to perform that function are used. Where the polynucleotides are to be used in a scheme which requires that a complementary strand be formed to a given polynucleotide, nucleotides are used which permit such formation.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. Abasic sites may be incorporated which do not prevent the function of the polynucleotide. Some or all of the residues in the polynucleotide can optionally be modified in one or more ways.

Standard A-T and G-C base pairs form under conditions which allow the formation of hydrogen bonds between the N3—H and C4-oxy of thymidine and the NI and C6—NH2, respectively, of adenosine and between the C2-oxy, N3 and C4—NH2, of cytidine and the C2—NH2, N′—H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610 to Collins et al. Other nonnatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione. Other such modified nucleotidic units which form unique base pairs are known, such as those described in Leach et al. (1992) J. Am. Chem. Soc. 114:3675-3683 and Switzer et al., supra.

The phrase “DNA sequence” refers to a contiguous nucleic acid sequence. The sequence can be either single stranded or double stranded, DNA or RNA, but double stranded DNA sequences are preferable. The sequence can be an oligonucleotide of 6 to 20 nucleotides in length to a full length genomic sequence of thousands of base pairs.

A “library of DNA sequences” refers to a plurality of DNA sequences. The number of “members of the library” is not critical; it can range from less than ten to greater than 10⁶. Typically in a library of DNA sequences, the library contains many different DNA sequences, all derived from the same parent DNA sequence but containing mutations in the sequence. The phrase “creating a library of DNA sequences” refers to the physical generation of a library of DNA sequences. Techniques used to physically generate a library are well know in the art and are referenced below. Typically, a “phage library” is created. “Phage libraries” comprise a DNA library incorporated into bacteriophage. The library is constructed such that the proteins encoded by the DNA library are expressed on the surface of the phage and thus on the surface of infected bacteria. The bacteria which contains the library is then “screened” for the presence of proteins with desired functionality. A “second library” is a library of DNA sequences based on the results found in the first library of DNA sequences. For example, if a beneficial mutation is found in the screening of a library, the mutation may be incorporated into the protein upon which the second library is based.

The term “IRL” refers to an information-rich library such as produced by a method of the invention.

The term “protein” refers to contiguous “amino acids” or amino acid “residues.” Typically, proteins have a function. However, for purposes of this invention, proteins also encompasses polypeptides and smaller contiguous amino acid sequences that do not have a functional activity. The functional proteins of this invention include, but are not limited to, esterases, dehydrogenases, hydrolases, oxidoreductases, transferases, lyases, and ligases. Useful general classes of enzymes include, but are not limited to, proteases, cellulases, lipases, hemicellulases, laccases, amylases, glucoamylases, esterases, lactases, polygalacturonases, galactosidases, ligninases, oxidases, peroxidases, glucose isomerases and any enzyme for which closely related and less stable homologs exist. In addition to enzymes, the encoded proteins which can be used in this invention include, but are not limited to, transcription factors, antibodies, receptors, growth factors (any of the PDGFs, EGFs, FGFs, SCF, HGF, TGFs, TNFs, insulin, IGFs, LIFs, oncostatins, and CSFs), immunomodulators, peptide hormones, cytokines, integrins, interleukins, adhesion molecules, thrombomodulatory molecules, protease inhibitors, angiostatins, defensins, cluster of differentiation antigens, interferons, chemokines, antigens including those from infectious viruses and organisms, oncogene products, thrombopoietin, erythropoietin, tissue plasminogen activator, and any other biologically active protein which is desired for use in a clinical, diagnostic or veterinary setting. All of these proteins are well defined in the literature and are so defined herein. Also included are deletion mutants of such proteins, individual domains of such proteins, fusion proteins made from such proteins, and mixtures of such proteins; particularly useful are those which have increased half-lives and/or increased activity.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides contain co- and/or post-translational modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations or esterifications of carboxyl groups) and the like are included within the meaning of polypeptide.

“Amino acids” or “amino acid residues” may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Variants of a protein” are those proteins that are related to one another by a common amino acid sequence or “parental protein” but contain minor variations in amino acid sequence from each other. These changes can be conservative substitutions, non-conservative substitutions, deletions, insertions or substitutions with non-naturally occurring amino acids (mimetics). The phrase “optimizing a protein” refers to the process of changing a protein to protein variants so that the desired functionality is improved. One of skill will realize that optimizing a protein could involve selecting a variant with lower functionality than the parental protein if that is desired.

The terms “aptamer” and “nucleic acid antibody” are used herein to refer to a single- or double-stranded polynucleotide that recognizes and binds to a desired target molecule by virtue of its shape. See, e.g., PCT Publication Nos. WO 92/14843, WO 91/19813, and WO 92/05285.

“Conservative residues” are those amino acid residues that have a similar property, such as similar chemistry. Conservative changes can be based, for example, on similar hydrophobicity, similar hydrophilicity, similar charge, similar propensity for adopting a particular secondary structure, similar shape, etc. Conservative substitution tables providing functionally similar amino acids are known in the art. In one scheme, the following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see, e.g., Creighton, Proteins (1984)).

“Amino acid mutations” are substitutions, deletions or insertions in amino acid sequences. For example, if an alanine occurs in an amino acid sequence, the alanine could be substituted to a serine, it could be deleted or another amino acid residue could be inserted on the amino or carboxy side of the residue. Because alanine and serine are members of the same conserved family of amino acids in the scheme described above, such a substitution can be termed a “conservative substitution.” Other schemes can be used.

The term “antibody” as used herein includes antibodies obtained from both polyclonal and monoclonal preparations, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, for example, Riechmann et al. (1988) Nature 332:323-327; Verhoeyan et al. (1988) Science 239:1534-1536; and U.K. Patent Publication No. GB 2,276,169, published Sep. 21, 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human hybridomas or from murine hybridomas made from mice expression human immunoglobulin chain genes or portions thereof. See, e.g., Cote, et al. Monoclonal Antibodies and Cancer Therapy, Alan R. Liss, 1985, p. 77.

The term “sequence alignment” refers to the result when at least two amino acid sequences are compared for maximum correspondence, as measured using one of the following “sequence comparison algorithms.” Optimal alignment of sequences for comparison can be conducted by any technique known or developed in the art, and the invention is not intended to be limited in the alignment technique used. Exemplary alignment methods include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), and by inspection.

The “three dimensional structure” of a protein is also termed the “tertiary structure” or the structure of the protein in three dimensional space. Typically the three dimensional structure of a protein is determined through X-ray crystallography and the coordinates of the atoms of the amino acids determined. The coordinates are then converted through an algorithm into a visual representation of the protein in three dimensional space. From this model, the local “environment” of each residue can be determined and the “solvent accessibility” or exposure of a residue to the extraprotein space can be determined. In addition, the “proximity of a residue to a site of functionality” or active site and more specifically, the “distance of the α or β carbons of the residue to the site of functionality” can be determined. (For glycine residues, which lack a β carbon, the α carbon can be substituted.) Also from the three dimensional structure of a protein, the residues that “contact with residues of interest” can be determined. These would be residues that are close in three dimensional space and would be expected to form bonds or interactions with the residues of interest. And because of the electron interactions across bonds, residues that contact residues in contact with residues of interest can be investigated for possible mutability. Additionally, molecular modeling can be used to determine the structure, and can be based on a homologous structure or ab initio. Energy minimization techniques can also be employed.

Although not dependent on three dimensional space, the “residue chemistry” of each amino acid is influenced by its position in a protein. “Residue chemistry” refers to characteristics that a residue possesses in the context of a protein or by itself. These characteristics include, but are not limited to, polarity, hydrophobicity, net charge, molecular weight, propensity to form a particular secondary structure, and space filling size.

The phrase “probability matrix” refers to a matrix for determining the probability that an amino acid can be substituted with another amino acid. Typically this matrix is in the form of an algorithm that determines the probability of substitution from the amino acid and its position. The individual entries in the matrix give a probability for placing a given amino acid in the preselected reference sequence at that position. The algorithm can be based on maintenance of structure, evolutionary diversity amongst a family of proteins and/or other factors described herein, as well as combinations thereof. The phrase “generating a probability matrix” refers to the process of determining the variable upon which the probability matrix will be based and, if needed, developing the algorithm to determine the substitutions in the matrix. The probability matrix can be “normalized” by setting the probability of a particular substitution in the matrix to “1” and correspondingly adjusting the relative probabilities of the other amino acids. The matrix can be normalized to the substitution most favored at that position by the algorithm, or to the value in the matrix for the wild type residue in the reference sequence at that position, or in any other desired manner. Normalization can be desirable to increase the degree to which mutations at a given position are sampled in generating the library.

The phrase “constraint vector” refers to a constraint put on or “applied to” the probability matrix to determine whether and the degree to which mutations at a given position in the matrix are to be included in the library. It too is typically an algorithm that determines whether a particular mutation will result in a functional protein. Variables that can be used to determine the constraint vector are also described below.

II. PROBABILITY MATRIX

A probability matrix is generated to provide an estimate that a given residue will provide a desired activity in a biological polymer of interest. The biological polymer can be a polynucleotide having its own activity of interest, or can encode a protein having an activity of interest. Biological polymers can include polynucleotides exhibiting catalytic activity, for example ribozymes, polynucleotides exhibiting binding activity, for example aptamers, polynucleotides exhibiting promoter activity, or polynucleotides exhibiting any other desired activity, alone or in combination with any other molecule.

The matrix comprises rows representing a given position in the biological polymer of interest, and columns for a plurality of different residues which can be incorporated into the reference sequence. The matrix entries give an estimate for the probability that incorporation of the residue in that column at the position in that row will produce a polymer having the desired activity.

A probability matrix can be generated for at least 2, 3, 4, 5, 6, 7, 8, 9, 10, 11, 12, 13, 14, 15, 16, 17, 18, 19, 20, 25, 30, 35, 40, 45, 50, 60, 70, 80, 90 or 100 positions in the reference sequence up to the entire sequence, and can include contiguous residues or noncontiguous residues or mixtures thereof. The matrix can include at least 2, 3, 4, 5, 6, 7, 8, 9, 10, 11, 12, 13, 14, 15, 16, 17, 18, 19, 20, 25, 30, 35, 40, 45 or 50 different residues. Naturally occurring residues can be included in the matrix, as well as unnatural residues for synthetic methods, and combinations thereof.

A profile can be created from the matrix based on probability scores and weighting factors. The probability matrix for a protein is preferably an n×20 matrix that calculates the probability for any point mutation of the target gene that the mutation will result in a protein having the desired function.

In one aspect, a probability matrix is calculated for a given protein library to be produced. To do this, numerical values are assigned to each amino acid that can be substituted into the sequence. One of skill will realize these numbers are arbitrary in that they are relative to each other only for the particular library being produced. It can be useful in some instances to assign the wild type residue at a given position a value of 1, although the wild type residue can be assigned any value. From this initial value, the values of each of the 20 encoded naturally occurring amino acids at each position can be assigned.

In some instances, it can be useful to assume, initially, that the wild type residue is a useful residue and results in a functional molecule. Thus, the value of most other residues should be less than that given to the wild type, therefore in the present example, less than “1”. Furthermore, in assigning values, residues that exhibit a low degree of conservation in homologs can be given large values in the probability matrix. Also, because areas of a protein which allow an insertion should be more tolerant to substitution, higher probabilities can be given to nonnative residues at positions which are close to insertions or deletions in homologs.

An example of a ranking of amino acid for valuation in this invention can be found in Gribskov, Proc Nat'l Acad Sci USA 84:4355 (1987). The degree of conservation for each position can be used to scale the values according to Gribskov.

Other information can be used to generate a probability matrix. For example, structural information has been found to be useful. As is well known, Hidden Markov models calculate the probability of going from one residue to the next based on sequence alignments. These models also include probabilities for gaps and insertions. See, Krogh, “An introduction to Hidden Markov models for biological sequences,” in COMPUTATIONAL METHODS IN MOLECULAR BIOLOGY, Salzberg, et al., eds, Elsevier, Amsterdam.

Other structural information found to be useful is the three dimensional structure of the protein. See for example, Dahiyat & Mayo, Protein Sci. 5:895 (1996). This can be determined crystallographically or from molecular modeling techniques. Energy minimization methods can also be employed.

A variety of different substitution matrices can be used as input for the calculation of a probability matrix. The choice of substitution matrix will impact the probability and ultimately the mutagenesis scheme. Thus, if mutations based on sequence alignment are desired, a sequence alignment substitution matrix should be chosen. Alternatively, if mutations that depend on general mutability are desired, a substitution matrix reflecting this need should be chosen.

Substitution matrices can be calculated based on the environment of a residue, e.g., inside or accessible, in α-helix or in β sheet. See, Overington, et al., Protein Sci 1:216 (1992). Methods to determine solvent accessible residues are known in the art. See, for example, Hubbard, Protein Eng 1:159 (1987).

More complex substitution matrices which consider secondary structure, solvent accessibility, and the residue chemistry are also suitable for use in probability matrices. See, for example, Bowie & Eisenberg, Nature 356:83 (1992).

One of skill will realize that a probability matrix can require quite complex mathematical calculations and therefore an algorithm that determines the matrix can be desired or even required. The development of such an algorithm is within the skill in the art following the teachings herein. Similarly, because of the complex calculations necessary to carry out the algorithm, it can be desirable to generate a computer program and employ it on a computer to calculate the probability matrix. Again, this is within the skill in the art.

III. CONSTRAINT VECTORS

The constraint vector preferably should reflect the likelihood that a specific mutation at each amino acid position of a protein will improve or affect the desired function of that protein. One example of a constraint vector is a correlation matrix. The constraint vector can also include knowledge-based component(s), such as prior knowledge of effects of single mutations, for example from mutagenesis scans or from naturally occurring mutations which affect the function of interest.

Another example is based on proximity. For example, it can be assumed that residues which are close to the active site of an enzyme are more likely to affect enzyme activity and/or specificity than more distant residues and thus, a mutation of a residue near the active site will affect the activity and/or specificity (either positively or negatively) than a mutation further away from the active site. The same proximity argument can be used for other applications: proximity to an epitope, proximity to an area of structural conflict, proximity to a conserved sequence, proximity to a binding site, proximity to a cleft in the protein, proximity to a modification site, etc.

There are a variety of methods available to estimate distance, and any technique known or developed in the art for estimating such distances can be used. For instance, the library can be constrained by distance of α or β-carbons to the active site of an enzyme. In another embodiment, the constraint can be based on the residues that make contact with the residues of interest (═1^{st} shell) and residues which contact the residues in the 1^{st} shell (═2^{nd} shell).

In another example, the simple distance function between β carbons of the enzyme and the β carbon of a bound ligand can be used to constrain a library. A linear function can be used where the threshold of acceptable mutations depends on the distance from the bound ligand. However, one can also utilize a variety of other functional relationships between distance and threshold of mutability, e.g., the square of the distance or the square root of the distance.

The physical distances from a known crystal structure of the reference sequence can be used. Alternatively, molecular modeling approaches can be used. For example, the structure of the reference sequence can be predicted based on its homology to a known structure, and then used to calculate distances. Or the entire structure of the reference sequence can be predicted and distances then calculated from the predicted structure. Energy minimization methods can be used.

Another way to generate constraint vectors is through correlation in evolutionary data. It has been observed that the replacement of a residue in a protein or protein family can be correlated with replacements in other positions. See, Lockless & Ranganathan, Science 286:295 (1999); and Gobel, et al., Proteins 18:309 (1994). In such cases it maybe advantageous to design the constraint vector such that all correlated residues are mutated simultaneously.

Conservation Indexes can be used as the elements of a constraint vector. In this capacity, one can avoid mutating residues that are highly conserved, or conversely, focus mutations on conserved regions of the protein. Algorithms for calculating Conservation Indexes at each position in a multiple sequence alignment are known in the art (Novere et al. Biophys. Journal v.76, p. 2329-2345, May 1999).

One of skill will realize that, like a probability matrix, generation of a constraint vector can require quite complex mathematical calculations and therefore an algorithm that determines the vector may be desired or even needed. The development of such an algorithm is within the skill in the art following the teachings herein. Similarly, because of the complex calculations necessary to carry out the algorithm, it can be desirable to generate a computer program and to employ it on a computer to generate the constraint vector. Again, this is within the skill in the art following the teachings herein.

IV. APPLICATION OF THE CONSTRAINT VECTOR TO THE PROBABLILITY MATRIX TO PRODUCE A SUBSTITUTION SCHEME

To determine which positions are to be permuted and which new residues will be tried in those positions, the constraint vector is applied to the probability matrix. This is done to increase the chance of finding improved variants and to decrease the risk of producing mutants with undesired properties, while generating a library of a size which can be effectively screened for a desired property. This application can also determine the degree to which a given change will be represented in the library, or a simpler threshold approach can be used, wherein all changes at a given position which meet the criteria imposed by the constraint vector are equally represented in the library.

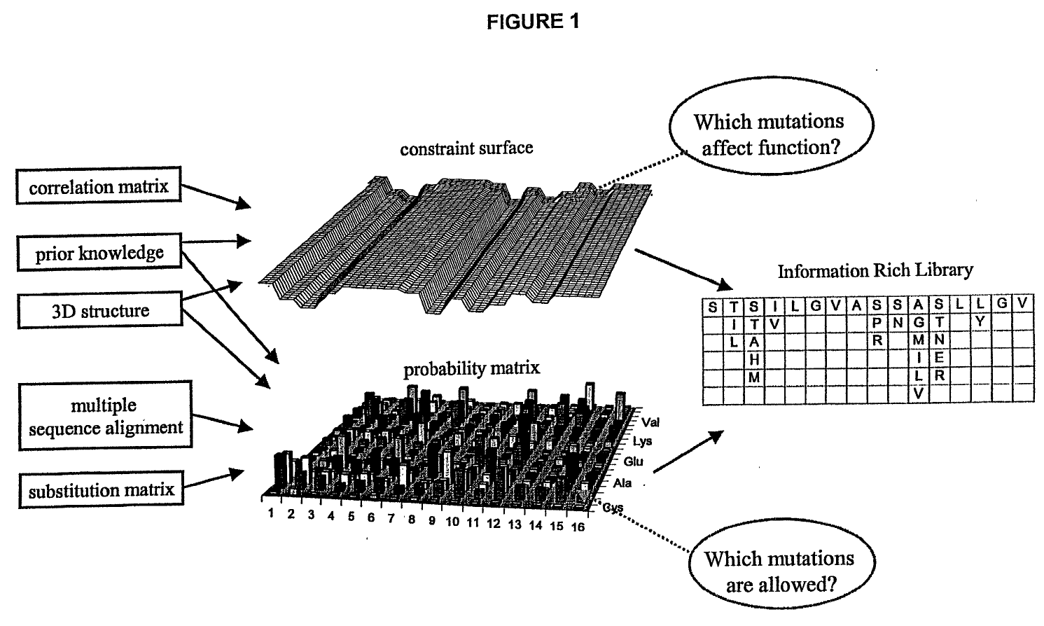

An exemplary algorithm is shown in FIG. 1. As is graphically represented in FIG. 1, the constraint vector can be imagined as being “lowered” onto the probability matrix. Positions in the probability matrix which are higher than the corresponding value in the constraint vector (i.e., which exceed the threshold imposed by the constraint vector) are candidates for mutagenesis. As the constraint vector is lowered, the number of positions to be mutagenized increases, and the number of new substitutions at each position increases. The degree to which the constraint vector is lowered is thus a determining factor in the size of the library which results. Application of the constraint vector can thus itself be constrained by the desired size of the library; a predetermined library size can be used to determine the degree to which the constraint vector allows the probability matrix to be sampled.

The substitution scheme produced by applying the constraint vector to the probability matrix is itself a useful result. The substitution scheme can be provided and used to create a library. The substitution scheme can be subjected to additional constraints prior to being employed in creating a library. For example, knowledge-based approaches can incorporate information about the activity of the polymer of interest and can be used to focus the substitution scheme to identify residues more likely to result in the desired activity when substituted as well as in identifying residues less likely to result in the desired activity.

One of skill will realize that the application of a constraint vector to a probability matrix can require quite complex mathematical calculations and therefore an algorithm that applies these two algorithms may be desired or required. The development of such an algorithm is within the skill in the art following the teachings herein. Similarly, because of the complex calculations necessary to carry out the application algorithm, it can be desirable to generate a computer program and employ it on a computer to do this. Again, this is within the skill in the art following the teachings herein.

V. CONSTRUCTION OF A LIBRARY

The simplest randomization scheme for polynucleotides encoding proteins is codon-based mutagenesis. In other words, after the amino acid residues to be mutated have been identified, the corresponding codons in the corresponding DNA sequence are randomized to create a DNA library. Procedures to randomize codons are known in the art (Huse et al., Int Rev Immunol. 1993;10(2-3):129-37; Kirkham et al., J Mol Biol. 1999 Jan 22;285(3):909-15). As one of skill will appreciate, more complicated randomization schemes can be designed which are more compatible with nucleotide-based mutagenesis.

Codon mutagenesis can be done in equimolar ratios, e.g., for a given site all mutagenic oligomers are added in equimolar ratios, or in ratios that relate to the probability matrix and/or the constraint vector. For example, one can bias a library in favor of mutations which are more likely to result in a functional protein. If desired, wild type oligos can be added to adjust the overall frequency of mutagenesis for a position or a region of the target gene.

In one embodiment, nucleotide-based randomization is used. This method has two advantages over synthesizing individual oligos for each substitution: it is less expensive as fewer oligos are needed; and the library will contain clones where neighboring (in linear sequence) positions have been simultaneously mutated.

Nucleotide-based mutagenesis can be optimized to produce a desired set of amino acids (Goldman & Youvan, Bio/Technology 10:1557 (1992); Huang & Santi, Anal Biochem 218:454 (1994); Jensen, et al., Nucleic Acids Res 26:697 (1998); and Tomandl, et al., J. Comp.-Aided Molec. Design 11: 29 (1997)). These authors did not consider a probability matrix; their focus was on inclusion of a desired set of amino acids. Nucleotide mixtures which encode amino acids mixtures that optimally conform to the calculated probability matrix and constraint vector can be calculated and synthesized.

Alternatively, portions of a coding region or an entire coding region can be chemically synthesized in a codon-by-codon technique using mixtures of activated trinucleotides at the positions to be substituted. In this way, only the desired codons are incorporated, dysfunctional mutations inevitably resulting from nucleotide-based randomization are avoided, and mixtures of adjacent changes can be readily provided. Additionally, controlling the degree of incorporation of a given mutation at a given position can be readily accomplished by varying the amount of the particular activated trinucleotides in the mixture for that position.

Oligonucleotide-driven site-directed mutagenesis can also be used. Suitable site-directed techniques include those in which a template strand is used to prime the synthesis of a complementary strand lacking a modification in the parent strand, such as methylation or incorporation of uracil residues; introduction of the resulting hybrid molecules into a suitable host strain results in degradation of the template strand and replication of the desired mutated strand. See Kunkel, Proc Natl Acad Sci U S A 1985 Jan;82(2):488-92; QuikChange™ kits available from Stratagene, Inc., La Jolla, Calif. Mixtures of individual primers for the substitutions to be introduced can be simultaneously employed in a single reaction to produce the desired combinations of mutations. Simultaneous mutation of adjacent residues can be accomplished by preparing a plurality of oligonucleotides representing the desired combinations. PCR methods for introducing site-directed changes can also be employed.

Oligos synthesized from mixtures of nucleotides can be used. The synthesis of oligonucleotide libraries is well known in the art. In one alternative, degenerate oligos from trinucleotides can be used (Gaytan, et al., Chem Biol 5:519 (1998); Lyttle, et al., Biotechniques 19:274 (1995); Virnekas, et al., Nucl. Acids Res 22:5600 (1994); Sondek & Shortle Proc. Nat'l Acad. Sci. USA 89:3581 (1992)). In another alternative, degenerate oligos can be synthesized by resin splitting (Lahr, et al., Proc. Nat'l Acad. Sci. USA 96:14860 (1999); Chatellier, et al., Anal. Biochem. 229:282 (1995); and Haaparanta & Huse, Mol Divers 1:39 (1995))

After the oligos which incorporate desired protein mutations are constructed, they can be assembled with the DNA that encodes the desired protein. Site-directed mutagenesis using a single stranded DNA template and mutagenic oligos is well known in the art (Ling & Robinson, Anal Biochem 254:157 (1997)). It has also been shown that several oligos can be incorporated at the same time using these methods (Zoller, Curr Opin Biotechnol 3: 348 (1992)). Single stranded DNA templates are synthesized by degrading double stranded DNA (Strandase™ by Novagen). The resulting product after strain digestion can be heated and then directly used for sequencing. Alternatively, the template can be constructed as a phagemid or M13 vector. Other techniques of incorporating mutations into DNA are known and can be found in, e.g., Deng, et al., Anal Biochem 200:81 (1992)). In an alternative embodiment, sequences are assembled by PCR fusion from synthetic oligos (Horton, et al., Gene 77:61 (1989); Shi, et al., PCR Methods Appl. 3:46 (1993); and Cao, Technique 2:109 (1990)). PCR with a mixture of mutagenic oligos can be used to create the DNA sequences that reflect the diversity of the library.

Cassette mutagenesis can also be used in site-directed random mutagenesis. Using this technique, a library can be generated by ligating fragments obtained by oligosynthesis, PCR or combinations thereof. Segments for ligation can, for example, be generated by PCR and subsequent digestion with type II restriction enzymes. This enables introduction of mutations via the PCR primers. Furthermore, type II restriction enzymes generate non-palindromic cohesive ends which significantly reduce the likelihood of ligating fragments in the wrong order. Techniques for ligating many fragments can be found in Berger, et al., Anal Biochem 214:571 (1993); and U.S. patent application Ser. No. 09/566,645, filed May 8, 2000.

A problem encountered in random mutagenesis is the manufacture of stop codons at the site of diversity. In vitro translation can be used to obtain libraries that are free of stop codons or other artifacts (Cho, et al., J Mol Biol 297:309 (2000)).

The particular chemical and/or molecular biological methods used to construct the library are not critical; any method(s) which provide the desired library can be used. For example, oligonucleotides can be inserted into a phage vector so that the phage particle expresses the encoded protein on its surface. Alternatively, one can manufacture a protein array wherein the encoded proteins are immobilized on a suitable surface and functional activity is assessed and the corresponding protein identified. In yet another embodiment, if the ability of a protein to bind to a target is the desired function, a mixture of proteins encoded by the library can be contacted with the desired target and the proteins bound identified and sequenced. For construction of libraries see, U.S. Pat. Nos. 6,114,149; 6,107,059; 5,922,545; 5,830,721; 5,723,323; 5,698,426; 5,571,698; 5,565,332; and PCT Patent Application WO 0046344.

VI. CHARACTERIZING THE LIBRARY MEMBERS

After a library is generated, the members can be characterized and the library screened for members that exhibit the desired activity. In addition to finding the desired functional protein, the information from the screen can be used to design improved probability matrix and constraint vectors for a next iteration of mutagenesis and library construction. For example, the probability matrix can be improved by determining the mutations in the gene that are compatible with expression, folding, and/or stability. Identifying stabilizing mutations or combinations of mutations can be of particular importance if library size is very limited by expense or difficulties in cloning. Under these conditions it can be advantageous to sequence all or most clones in a library. In a subsequent round of evolution the deleterious mutations identified in the prior round can then be avoided altogether. In addition, all of the sequences present in the library can be sequenced if the number of clones to be assayed is small. It can be cost efficient to sequence even clones which have no activity because they help to improve the probability matrix. Sequencing using DNA or RNA arrays (Hyseq, Inc.) can be used.

After screening for a particular function, it can be determined which mutations affect that function. This information would help to understand the underlying mechanism of the functional protein. Furthermore, the next round of library construction can be focused on these positions and neighboring residues which produce the desired activity (i.e., the constraint vector can be modified to better ensure functional proteins). The constraint vector can also be improved by determining the combinations of mutations that occur simultaneously in improved clones. These residues may interact and should be mutated simultaneously in subsequent rounds. Such synergistic mutations can be particularly important because they are almost impossible to identify by simple random mutagenesis.

Analysis of the library can also reveal the mutations that are missing from the unselected libraries. This could indicate toxicity, in addition to technical problems with library construction. If it is determined that an individual clone is toxic, such a polynucleotide or its encoded protein may find use as a drug or compound in which toxicity to bacteria is desired (assuming the library is constructed in E. coli). A related issue is the fitness distribution in the library. This can indicate the optimum mutation frequency for the library. The fitness distribution can also be used to compare various methods of calculating the probability matrix and the constraint vector, i.e., the presence of continuous improvements of these methods.

Other useful products produced by the method of the invention include polynucleotides incorporating mutations identified through construction and screening of such libraries, vectors (including expression vectors) comprising such polynucleotides, host cells comprising such polynucleotides and/or vectors, and libraries of biological polymers, and libraries of host cells comprising and/or expressing such libraries of biological polymers.

VII. CORRELATION BETWEEN STRUCTURE AND FUNCTION OF PROTEIN MUTANTS

Statistical analyses of the correlation between structures and functions of molecules have been widely used to guide the optimization of small molecule drugs (quantitative structure activity relationship, or QSAR). One can differentiate between parameter-free approaches (for example Free, J. Med. Chem. (1964)) and methods which consider various physico-chemical parameters of the various substituents of a molecule (for example Carotti, Chem Biol Interact 67:171 (1988)). See also, Goldman, et al., Drug Development Research 33:125(1994) and Lahr, et al., Proc. Nat'l Acad. Sci. USA 96:14860 (1999). Either approach can be used for the libraries of the instant invention. In addition one can use algorithms based on the 3D structure of the protein of interest.

The amino acid sequence can be determined for variants that exhibit desired properties. The variants may each contain multiple mutations with respect to the parent molecule, and several variants may share one or more identical mutations while having other, nonshared mutations. The data mining task is to assign the degree to which individual mutations or combinations of mutations contribute to the observed improvement in properties, and to identify which pairs or groups of amino acids interact with each other (i.e. the observed measured property for the combined mutations is non-additive compared to the effect of the mutations individually). Methods for performing this data mining are known in the art; computer programs implementing suitable techniques are available (e.g., Spotfire).

VIII. CO-VARIATION AS A TOOL TO SELECT THE REGION TO BE MUTAGENIZED

Co-variation is the tendency of some residues to change simultaneously with other residues, i.e., the residues are linked during evolution. These co-variant residues can be linked by structure and/or they may be linked by function. Once coupled residues have been identified, if one of the residues is found to be a candidate for mutation, the other residue can be assigned a higher probability of being a candidate as well. In this way, mutations which otherwise would not be obvious in a probability matrix or a constraint vector can be included. For further discussion of co-variation, see Gobel, et al., Proteins 18:309 (1994); Jespers, et al., J. Mol. Biol. 290:471 (1999); and Pazos, et al., Comput. Appl. Biosci. 13:319 (1997).

VI. UTILITY OF THE LIBRARIES OF THIS INVENTION

While the utility of the libraries of this invention will be evident to one of skill in the art, the libraries will be particularly useful in preparation of enzymes or ligands with increased activity, enzymes or ligands with modified activity, proteins with increased stability, removal of immunogenic epitopes from useful proteins, improving expression levels of proteins, and improving grafting of domains or loops into proteins.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

### Example 1

### Subtilisin With Novel Substrate Specificity

GG36 (savinase) is a subtilisin protease from Bacillus lentus. The goal of this Example is to generate mutants of the protease that possess a novel substrate specificity.

A published multiple sequence alignment of 124 subtilisin-like serine proteases (Siezen, et al., Protein Science 6:501 (1997)) was recreated from a publicly available database (GENBANK), with the sequence labeled baalkp in the database being substituted with that of GG36. GG36 differs from baalkp by only one residue substitution. In baalkp, residue 87 is an asparagine while in GG36 a serine residue is found at the corresponding position. The GG36 amino acid sequence was used as the reference sequence, and those positions of the alignment for which the GG36 sequence had a gap character were deleted.

A profile for the alignment was generated using the method of Gribskov (Gribskov, Proc. Nat'l Acad. Sci. USA 84:4355 (1987)) except that a mutation probability matrix was used in place of the log-odds matrix used by Gribskov. See Table 1. The mutation probability matrix gives the probabilities that a given amino acid will mutate to any another amino acid in a given evolutionary interval (Dayhoff, et al., Atlas of Protein Sequence and Structure (Natl. Biomed. Res. Found., Washington), Vol. 5, Suppl. 3, pp. 345-358 (1978)). The mutation probability matrix PAM 128, generated from the PAM1 matrix as described by Dayhoff, was used.

In the Gribskov method, the value of the profile for amino acid a at position p is given by

$$M ( p , a ) = {\sum }_{b = 1}^{20} W ( p , b ) \times Y ( a , b )$$

where Y(a,b) is the probability obtained from Dayhoff's mutation probability matrix for the substitution of a for b, and W(p,b) is a weight for amino acid b at position p.

The frequency of an amino acid in the alignment at a particular position was used for its weight:

W(b,p)=n(b,p)/Nᵣ,

where n(b,p) is the number of times b appears at position p, and Nᵣ is the total number of amino acid counts at that position.

The probability matrix, GG36 residues against all 20 substitution residues, was normalized to the largest fraction in each row. See Table 2.

The constraint vector was designed such that mutagenesis would focus on positions which are close to the active site of the enzyme. The calculation was based on two crystal structures which have peptides bound to different regions of the active site: a structure of FN2 (a subtilisin mutant from B. lentus, which is identical to GG36 except for the following substitutions; K27R, V104Y, N123S, and T174A) which contained the peptide Ala-Ala-Pro-Phe bound to the S₄ to S₁ subsites; and a structure of subtilisin BPN′ (from B. amyloliquefaciens) which had the inhibitor Suc-Ala-Phe-Ala bound to the S′₁, to S′₃ subsites. Both structures were aligned using the program “insight II” (MSI, San Diego, Calif.). Subsequently, the coordinates of the inhibitor Suc-Ala-Phe-Pro-Ala were moved into the structure of FN2. The combined coordinates were imported into Excel (Microsoft, Redmond, Wash.). For each residue of the enzyme the distance between the beta carbon atom and the closest beta carbon atom of the two bound peptides was calculated. Where glycine residues, which do not have a beta carbon, occurred, the distance between the alpha carbon of the glycine residue and the beta carbon of the bound peptide was calculated instead.

For each backbone residue, a selection value was calculated using the constraint vector as described below. This value was used to select residues from the sequence profile for inclusion in the substitution table. Profile values greater than or equal to the selection value were added to the substitution list for that position. The lower the value, the increased chance that a substitute residue was selected at that position.

A linear constraint vector of the formula y=mx+b was used to generate the combinatorial selection scheme, where x=Cβ min. The m and b terms were chosen to provide ˜100 substitutions from residues between 1 and 10 Å from the active site as described, yielding m=0.15500 and b=−0.40000. Any y values >1 (which result from a distance of>10 Å) were ignored. Entries in the profile shown in Table 1 which exceeded the y value determined for that position by applying the constraint vector (and <1) were selected for inclusion in the combinatorial library. Application of the constraint vector to the probability matrix in this manner produced the substitution table shown in Table 3, containing 105 suggested substitutions.

Visual inspection of the enzyme structure determined that most residues which are close to the bound ligand were included in the mutagenesis scheme. It was decided to avoid mutation of positions H62 and S215 as proposed by the algorithm because these two residues are part of the catalytic triad of subtilisin. Furthermore, V66C was eliminated from the mutagenesis scheme because an unpaired Cys residue is unlikely to lead to a functional GG36. These alterations represent contribution of a knowledge-based constraint to the results produced by applying the constraint vector to the probability matrix. As the consensus sequence derived from alignment of the large family was quite different from that of savinase, the most prevalent residue at several positions in the profile was not the residue in the savinase backbone. Additionally, in some cases the wild type residue was suggested to be substituted with itself. In cases where only a single substitution of a residue was suggested, the technique used to form the library could be doped with the wild type residue to prevent inclusion of a possibly debilitating residue in all members of the library.

### Example 2

### Alteration of β-lactamase Specificity Using a Scoring Profile

This example demonstrates the application of a distance-based constraint vector to a position-specific scoring matrix generated using a multiple sequence alignment of seven members of the ampC family of proteins and a PAM32 substitution matrix.

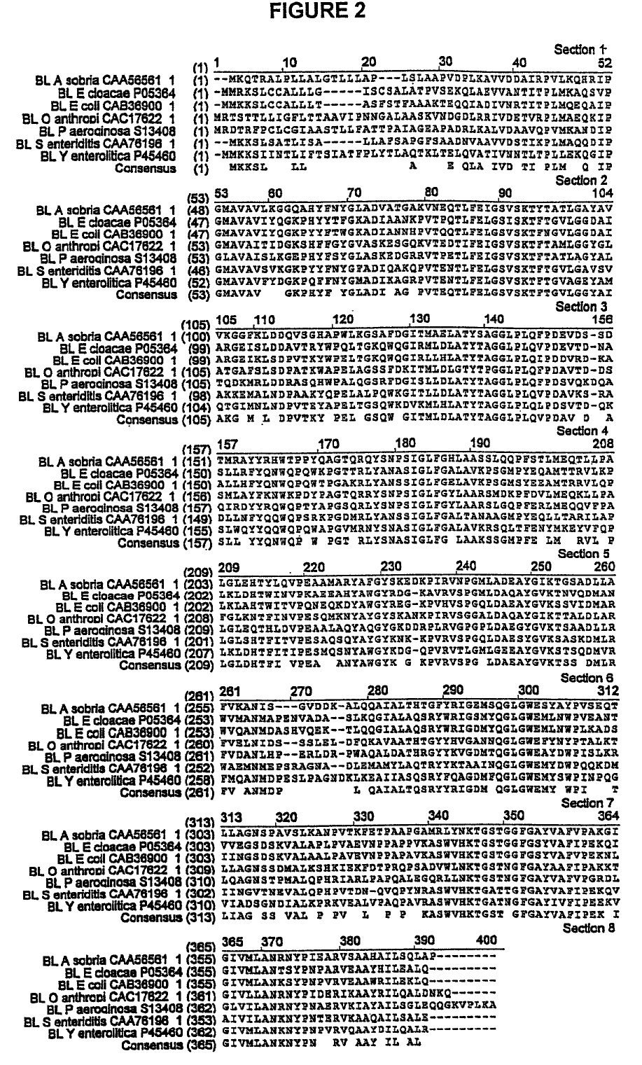

To create the IRL produced in this example, 7 beta lactamase ampC protein sequences (those from A. sobria, E. coli, O. anthropi, P. aeroginosa, S. enteriditis and Y. enterolitica) were aligned using the default parameters of the program AlignX (a component of Vector NTI Suite 6.0 from Informax, Inc.), which is an implementation of the ClustalW alignment algorithm [Thompson, J. D., D. G. Higgins, et al. (1994). Nucleic Acids Res 22(22): 4673-80.]. See FIG. 2. The sections of the alignment for which the reference sequence (E. cloaceae) had a gap character were discarded, as only positions at which the reference sequence contained an amino acid were used.

The multiple sequence alignment of ampC was used to generate a profile using the method of Gribskov as described above except that a mutation probability matrix was used instead of the log-odds substitution matrix form used by Gribskov. The mutation probability matrix gives the probabilities that any given amino acid will mutate to each of the other amino acids in a given evolutionary interval. The mutation probability matrix PAM 32, which was generated from the PAM1 matrix as described [Dayhoff, M. et al. (1978) Atlas of Protein Sequence and Structure (Natl. Biomed. Res. Found., Washington), Vol. 5, Suppl. 3, pp. 345-358)], was used.

A distance-based constraint was applied to the scoring matrix to limit mutations to residues that are surface exposed and within 6 angstroms from the binding site of ligands in the E. cloacae ampC 3D structure. Specifically, the E. cloacae ampC crystal structure (Protein Database Base ID# 1BLS) and 6 E. coli ampC structures containing bound inhibitors or substrates (Protein Database Base structures 1C3B, 1FCM, 1FCN, 1FCO, 1FSW, 1FSY) were first loaded into the program MOE 2000.01 (Chemical Computing Group, Inc., Montreal Canada). Because each structure consists of a homodimer, one of the monomers and its associated ligand was deleted. Next, the main chains of all the structures containing bound ligands were aligned (0.4 angstroms RMS deviation) and all the water molecules were manually deleted. The main chains of all structures except the E. cloacae structure (1BLS) were then removed. The resulting structure consisted of the E. cloacae ampC molecule with all of the superimposed ligands from the other 6 ampC structures. All surface-exposed side chains (i.e, the beta carbon and additional atoms not in the backbone) in ampC with atoms within 6 angstroms of the ligand atoms were then selected for the IRL library. Five of the top substitutions based on the scoring matrix were chosen at each of these sites. This library was termed the ‘profile library’ or IRL1 library.

To create the IRL1 DNA library, 90 mutagenic forward primers containing the different substitutions were designed and used in a PCR reaction containing a single wild type reverse primer and the E. cloacae ampC-containing plasmid pAL20 as template. After digestion of the methylated template DNA using the DpnI enzyme, the PCR product was used to transform E. coli. The transformants were plated on kanamycin plates to determine the number of transformants obtained or kanamycin plates containing different concentrations of moxalactam (mox) to obtain moxalactam resistant clones. The mox-resistant clones were further characterized to determine the fold increase in resistance compared to cells containing the wild type ampC gene. Ten mox-resistant clones were obtained, which had a fold increase in mox-resistance ranging from around 3-fold to 20-fold (0.8-6 μg/mL) above wild type (0.3 μg/mL).

Sequencing of the ampC gene in the plasmids from these variants revealed that each of them contained one to three of the selected library amino acid changes in ampC (Table 4). Two of the variants, IRL1.8.4 and IRL1.8.5 also contained additional mutations introduced during the PCR process (Table 4). The IRL1.6.1 variant, which has a 20-fold increase in mox-resistance was the best variant in this library and had two changes at positions S288 and R348. The substitutions Y220N, A219P and L61M appeared in more than one clone suggesting that they may be important for conferring resistance. Thus, this example shows that the application of a distance-based constraint onto a scoring matrix was successful in producing ampC variants that had a significantly higher resistance to the antibiotic moxalactam.

### Example 3

### Alteration of β-lactamase Specificity Using a Recruitment Matrix

This Example demonstrates the application of a distance-based constraint vector to the E. cloacae ampC molecule and recruitment of amino acids observed in other ampC proteins.

To create the IRL library in this example, first, the sequence of the ampC protein from E. cloacae (reference sequence) was aligned with ampC protein sequences from A. sobria, E. coli, O. anthropi, P. aeroginosa, S. enteriditis and Y. enterolitica using the AlignX program from Vector NTI Suite (Informax Inc. Bethesda, Md.). Those positions in the alignment where amino acids other than those found in the reference sequence were observed were recruited, and a distance-based constraint vector was applied to these positions to limit mutations to residues that were surface exposed and 6 angstroms from the binding site of ligands to the E. cloacae ampC 3-D structure. Specifically, the E. cloacae ampC crystal structure (Protein Database Base ID# 1BLS) and 6 E. coli ampC structures containing bound inhibitors or substrates (Protein Database Base structures 1C3B, 1FCM, 1FCN, 1FCO, 1FSW, 1FSY) were first loaded into the program MOE 2000.01 (Chemical Computing Group, Inc., Montreal Canada). Because each structure consists of a homodimer, one of the monomers and its associated ligand was deleted. Next, the main chains of all the structures containing bound ligands were aligned (0.4 angstroms RMS deviation) and all the water molecules were manually deleted. The main chains of all structures except the E. cloacae structure (1BLS) were then removed. The resulting structure consisted of the E. cloacae ampC molecule with all of the superimposed ligands from the other 6 ampC structures. All surface-exposed side chains (i.e, did not count the backbone, just the beta carbon, and outward atoms) in ampC with atoms within 6 angstroms of the ligand atoms were then selected for the IRL library. Eight positions were selected and substitutions were chosen based on the amino acids observed at those positions in other members of the ampC protein family used in the alignment. This library was termed the ‘recruitment library’ or IRL2 library.

To create the IRL2 DNA library, 15 mutagenic forward primers containing the different substitutions were designed and used in a PCR reaction containing a single wild type reverse primer and the E. cloacae ampC-containing plasmid pAL20 as template. After digestion of the methylated template DNA using the DpnI enzyme, the unmethylated PCR product was used to transform E.coli. The transformants were plated on kanamycin plates to determine the number of transfomants obtained or kanamycin plates containing different concentrations of moxalactam (mox) to obtain moxalactam resistant clones. The mox-resistant clones were further characterized to determine the fold increase in resistance compared to cells containing the wild type ampC gene. Fifteen mox-resistant clones were obtained, which had a fold increase in mox-resistance ranging from around 3 fold to 83 fold (0.8-25 μg/mL) above wild type (0.3 μg/mL) in a single round.

Sequencing of the ampC gene in the plasmids from these variants revealed that 12 variants contained one to three of the desired library amino acid changes in ampC (Table 4). In addition to the desired mutations observed in the winners, some of the winners had additional unexpected mutations which may have contributed to the phenotype in some cases. Four of the variants contained additional unexpected mutations either in the promoter or within the ampC gene due to errors in the PCR process. These included S263P in IRL1.8.4, S17T in the signal sequence in IRL1.8.5, A217V in IRL2.8.4, and T125M in IRL2.3.6. The observation that 3 of the 15 variants contained wild type ampC sequence suggests that mutations elsewhere in the plasmid vector or in the E. coli genome can contribute to the phenotype, which is not unexpected. Silent muations were also seen at position A351 in IRL1.8.10, S286 in IRL2.8.3, and at A152 in IRL2.8.14. Promoter region mutations were seen in IRL2.8.7 (a to g at +168), IRL2.8.12 (c to t at +136), and IRL2.8.13 (c to t at +237 and t to c at +205).

The substitutions V120F and N345I appeared in several clones suggesting their importance for increasing mox resistance. Although it can be argued that these mutations came up several times due to PCR primer bias, the sequencing of random library clones not selected for mox resistance did reveal other positions where a large number of substitutions were seen, but which did not show up in the variants. It is interesting that compared to the IRL1 library, the IRL2 library shows a different profile of substitutions in the variants. Again, this example shows that the use of a distance-based constraint and recruited residues from multiple sequence alignment were successful in producing ampC variants that had a significantly higher resistance to the antibiotic moxalactam.

Molecular Biological Methods

The mutagenic primers used for creating the PCR-based DNA libraries each contained 37 bases with 17 bases flanking the mutant codon on both sides. All mutagenic and wt primers used for creating the DNA libraries or for sequencing were obtained from Operon Technologies (Alameda, Calif.).

A single reverse primer and 90 IRL1 or 15 IRL2 mutagenic forward primers were used in a PCR reaction with a template, plasmid pAL20 containing the E. cloacae ampC gene. Plasmid pAL20 was created by sub-cloning the ampC gene into the TOPOBLUNT vector (kan^{y}) obtained from Invitrogen (Carlsbad, Calif.). The final reaction contained 0.5 μM of the reverse primer and 0.5 μM of all IRL forward primers combined (all primers together were 25 pmols), 16 fmol of pAL20, 15 nmol of each dNTPs, 5 units of the Herculase polymerase (Stratagene, La Jolla, Calif.) and a Herculase-specific buffer also from Stratagene. The total reaction volume was 100 μL. The cycling conditions included an initial cycle at 94° C. for 3 minutes followed by 30 cycles each containing a step at 94° C. for 30 seconds, a 55° C. step for 30 s and a 68° C. step for 5 minutes. A final elongation cycle at 68° C. for 7 minutes was also included. An MJ Research PTC thermal cycler was used for the PCR reaction. After the PCR reaction was carried out, the plasmid template in each of the PCR reactions was digested with the DpnI enzyme, which cleaves the methylated DNA template and not the PCR product.

For each library, 1 μL of the DpnI digested PCR reaction was transformed by electroporation into TOP10 one-shot electrocompetent cells from Invitrogen. The electroporation was conducted using a BIORAD electroporator. A fifth of the transformation mix was plated on LB plates containing 50 μg/mL kanamycin (kan) and the remaining mix was plated on LB plates containing 50 μg/mL kan and 0.5 μg/mL moxalactam (mox; obtained from Sigma). Between 2000 and 4000 transformants were obtained per transformation based on the number of colonies observed on the kan plates. Several transformations were carried out to obtain 21000 and 54000 colonies for the IRL1 and IRL2 libraries respectively. Those transformants that grew on plates containing mox were streaked for single colonies on LB plates containing 50 μg/mL kan and 0.5 μg/mL mox. A single colony from each of the mox-resistant clones was used to inoculate 200 μL of LB containing kan in a 96 well microtiter plate. The plate was grown at 37° C. with shaking for 18 hours, and each of the cultures in the wells was diluted 10,000-fold into 12 microtiter plates containing LB with different concentrations of mox (0 to 100 μg/mL). Kanamycin was also added to the media to maintain selection for the ampC pAL20 plasmid. After incubation at 37° C. with shaking for up to 21 hours, the absorbance of the cells grown in each well was measured at 600 nm. The fold increase in mox resistance was calculated based on the extent of growth of cells containing the wild type ampC gene. Plasmids were extracted for sequencing from all library clones that had a mox resistance of greater than 2.5 fold compared to wild type.

### Example 4

### Generation of a Conservation Index as a Constraint Vector

A conservation index may be defined as a measure of the degree of conservation at each position in a multiple sequence alignment. A conservation index algorithm developed by Novere et al. (Biophys. Journal v.76, p. 2329-2345, May 1999) was used to generate a conservation index based on the alignment of the ampC proteins. A conservation index was assigned at each position in the alignment according to the equation:

$$C I = \frac{{\sum }_{i = 1}^{N} {\sum }_{j = i + 1}^{N} \frac{{s}_{i j}}{{S}_{i j}}}{{\sum }_{i = 1}^{N} {\sum }_{j = i + 1}^{N} {S}_{i j}}$$

where N is the number of sequences in the alignment, Sᵢⱼ are the global similarities of the ith and jth sequences, and sᵢⱼ is the relevant similarity matrix element for the sequences i and j at the given position. The default similarity matrix from the Wisconsin package program GAP (Devereux et al., 1984) can be used, resealed to [0-100]. The resulting values range from 0 to 100. A score of 100 indicates absolute conservation.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

TABLE 1

| | | |
|---|---|---|
| residue | backbone | Profile |

| | | | | | | | | | | | | | | | | | | | | | |
|---|---|---|---|---|---|---|---|---|---|---|---|---|---|---|---|---|---|---|---|---|---|
| number | residue | A | C | D | E | F | G | H | I | K | L | M | N | P | Q | R | S | T | V | W | Y |

| | | | | | | | | | | | | | | | | | | | | | | |
|---|---|---|---|---|---|---|---|---|---|---|---|---|---|---|---|---|---|---|---|---|---|---|
| 1 | A | 0.1027 | 0.0119 | 0.0633 | 0.0521 | 0.0232 | 0.079 | 0.0367 | 0.0196 | 0.089 | 0.0412 | 0.0101 | 0.0764 | 0.063 | 0.0356 | 0.0428 | 0.1053 | 0.0797 | 0.0347 | 0.0249 | 0.0139 | |
| 2 | Q | 0.079 | 0.0055 | 0.1408 | 0.1131 | 0.0079 | 0.1013 | 0.0435 | 0.0154 | 0.0716 | 0.035 | 0.0083 | 0.0684 | 0.0502 | 0.0765 | 0.0402 | 0.0649 | 0.0428 | 0.0291 | 0.0015 | 0.007 | |
| 3 | S | 0.1357 | 0.0129 | 0.0443 | 0.0445 | 0.018 | 0.0742 | 0.021 | 0.0273 | 0.0468 | 0.0546 | 0.0089 | 0.0378 | 0.168 | 0.0343 | 0.0236 | 0.0964 | 0.0903 | 0.0557 | 0.0017 | 0.0138 | |
| 4 | V | 0.0766 | 0.0105 | 0.0342 | 0.0394 | 0.0596 | 0.0503 | 0.0298 | 0.0447 | 0.0889 | 0.1207 | 0.0202 | 0.0356 | 0.0399 | 0.035 | 0.0483 | 0.0611 | 0.0621 | 0.0864 | 0.0121 | 0.0488 | |
| 5 | P | 0.0891 | 0.0086 | 0.0468 | 0.0681 | 0.0104 | 0.0557 | 0.0408 | 0.023 | 0.0744 | 0.0471 | 0.02 | 0.0374 | 0.1602 | 0.1011 | 0.0395 | 0.0695 | 0.0619 | 0.0428 | 0.0014 | 0.0103 | |
| 6 | W | 0.0299 | 0.0058 | 0.017 | 0.0158 | 0.0392 | 0.0182 | 0.0164 | 0.0105 | 0.0314 | 0.037 | 0.0054 | 0.0193 | 0.0276 | 0.0135 | 0.0531 | 0.0407 | 0.0259 | 0.015 | 0.5164 | 0.0621 | |
| 7 | G | 0.0845 | 0.0105 | 0.0502 | 0.044 | 0.0458 | 0.228 | 0.0463 | 0.0154 | 0.0428 | 0.0382 | 0.0053 | 0.0441 | 0.0371 | 0.0283 | 0.0245 | 0.0668 | 0.0404 | 0.0341 | 0.003 | 0.1089 | |
| 8 | I | 0.0539 | 0.0079 | 0.0164 | 0.0203 | 0.073 | 0.0263 | 0.0135 | 0.083 | 0.0299 | 0.315 | 0.0365 | 0.0173 | 0.0363 | 0.0202 | 0.0156 | 0.0325 | 0.0401 | 0.1074 | 0.0019 | 0.056 | |
| 9 | S | 0.0873 | 0.0081 | 0.061 | 0.0632 | 0.0326 | 0.1097 | 0.0495 | 0.0235 | 0.0925 | 0.0692 | 0.0101 | 0.0532 | 0.0592 | 0.0521 | 0.0466 | 0.0709 | 0.0521 | 0.0391 | 0.0023 | 0.0218 | |
| 10 | R | 0.084 | 0.021 | 0.0502 | 0.0496 | 0.0215 | 0.0837 | 0.0406 | 0.0269 | 0.1008 | 0.055 | 0.0177 | 0.0481 | 0.0433 | 0.0487 | 0.1123 | 0.0724 | 0.0544 | 0.044 | 0.0049 | 0.0221 | |
| 11 | V | 0.0808 | 0.0113 | 0.0259 | 0.0306 | 0.034 | 0.0767 | 0.0126 | 0.1329 | 0.0416 | 0.1261 | 0.0199 | 0.0265 | 0.0322 | 0.0191 | 0.0257 | 0.0538 | 0.0729 | 0.1655 | 0.0014 | 0.0167 | |
| 12 | Q | 0.088 | 0.0119 | 0.0682 | 0.0668 | 0.0228 | 0.0955 | 0.0427 | 0.0205 | 0.0894 | 0.0373 | 0.0082 | 0.0601 | 0.0424 | 0.0609 | 0.0544 | 0.0905 | 0.0632 | 0.0331 | 0.0035 | 0.0426 | |
| 13 | A | 0.1268 | 0.0177 | 0.0389 | 0.04 | 0.042 | 0.0836 | 0.0174 | 0.0542 | 0.0451 | 0.0835 | 0.0167 | 0.0395 | 0.0388 | 0.0223 | 0.0201 | 0.0776 | 0.0874 | 0.125 | 0.0077 | 0.0208 | |
| 14 | P | 0.0825 | 0.0084 | 0.0788 | 0.0698 | 0.0189 | 0.0692 | 0.0278 | 0.0326 | 0.0962 0.0712 | 0.0156 | 0.0523 | 0.0765 | 0.0406 | 0.043 | 0.0719 | 0.066 | 0.0517 | 0.0197 | 0.0136 | | |
| 15 | A | 0.0829 | 0.018 | 0.0472 | 0.0609 | 0.0542 | 0.0718 | 0.0303 | 0.0252 | 0.0739 | 0.0366 | 0.0092 | 0.0353 | 0.0593 | 0.0481 | 0.0568 | 0.0631 | 0.0481 | 0.0441 | 0.0221 | 0.1131 | |
| 16 | A | 0.1291 | 0.0115 | 0.0391 | 0.0441 | 0.0234 | 0.0672 | 0.021 | 0.0577 | 0.051 | 0.1271 | 0.0177 | 0.0354 | 0.0437 | 0.0291 | 0.0299 | 0.0702 | 0.0659 | 0.1149 | 0.0018 | 0.0229 | |
| 17 | H | 0.0286 | 0.0159 | 0.019 | 0.0201 | 0.0791 | 0.0274 | 0.0529 | 0.013 | 0.0251 | 0.0448 | 0.0042 | 0.0242 | 0.017 | 0.0253 | 0.0382 | 0.033 | 0.0182 | 0.0181 | 0.3259 | 0.1655 | |
| 18 | N | 0.0958 | 0.0139 | 0.1085 | 0.097 | 0.01 | 0.0819 | 0.0294 | 0.0188 | 0.1015 | 0.0341 | 0.008 | 0.0592 | 0.0521 | 0.0528 | 0.0562 | 0.0742 | 0.06 | 0.0366 | 0.0024 | 0.0117 | |
| 19 | R | 0.0826 | 0.0086 | 0.0556 | 0.0619 | 0.0262 | 0.0552 | 0.0323 | 0.032 | 0.1122 | 0.0837 | 0.0313 | 0.0402 | 0.0394 | 0.06 | 0.0784 | 0.066 | 0.0606 | 0.0547 | 0.0034 | 0.0189 | |
| 20 | G | 0.108 | 0.0109 | 0.0559 | 0.0539 | 0.014 | 0.2357 | 0.019 | 0.0196 | 0.0633 | 0.0249 | 0.0063 | 0.0513 | 0.0532 | 0.0269 | 0.031 | 0.102 | 0.0716 | 0.038 | 0.0024 | 0.016 | |
| 21 | L | 0.0606 | 0.0156 | 0.0197 | 0.0233 | 0.1225 | 0.0411 | 0.0225 | 0.059 | 0.041 | 0.099 | 0.0126 | 0.026 | 0.026 | 0.0237 | 0.0262 | 0.0446 | 0.0409 | 0.0844 | 0.0053 | 0.2044 | |
| 22 | T | 0.1222 | 0.0117 | 0.0514 | 0.0475 | 0.0315 | 0.0969 | 0.0197 | 0.0268 | 0.083 | 0.0457 | 0.0102 | 0.0445 | 0.0448 | 0.03 | 0.0426 | 0.0984 | 0.1261 | 0.0511 | 0.0027 | 0.0209 | |
| 23 | G | 0.1124 | 0.0063 | 0.046 | 0.0405 | 0.0114 | 0.4286 | 0.0103 | 0.0102 | 0.0349 | 0.0176 | 0.0036 | 0.0391 | 0.0345 | 0.0178 | 0.0156 | 0.0881 | 0.0447 | 0.0299 | 0.0013 | 0.0086 | |
| 24 | S | 0.0882 | 0.01 | 0.0558 | 0.0655 | 0.0138 | 0.066 | 0.0335 | 0.0195 | 0.1622 | 0.0283 | 0.0102 | 0.0515 | 0.0425 | 0.0542 | 0.0928 | 0.0824 | 0.0668 | 0.0322 | 0.0099 | 0.0199 | |
| 25 | G | 0.102 | 0.0058 | 0.0645 | 0.0496 | 0.01 | 0.3527 | 0.0197 | 0.0116 | 0.0556 | 0.019 | 0.0043 | 0.0588 | 0.0307 | 0.0244 | 0.0227 | 0.0858 | 0.0484 | 0.0285 | 0.0013 | 0.0063 | |
| 26 | V | 0.0932 | 0.0139 | 0.0249 | 0.0302 | 0.0208 | 0.0468 | 0.0144 | 0.1192 | 0.0396 | 0.0964 | 0.0212 | 0.0249 | 0.0295 | 0.0273 | 0.0185 | 0.0563 | 0.0893 | 0.2275 | 0.0009 | 0.0108 | |
| 27 | K | 0.0749 | 0.0525 | 0.0273 | 0.0278 | 0.0273 | 0.0495 | 0.0231 | 0.0642 | 0.0882 | 0.0718 | 0.0149 | 0.0333 | 0.0295 | 0.0239 | 0.0741 | 0.0635 | 0.0793 | 0.1394 | 0.0036 | 0.0359 | |
| 28 | V | 0.104 | 0.0141 | 0.0211 | 0.026 | 0.0233 | 0.051 | 0.0106 | 0.1283 | 0.0312 | 0.1198 | 0.0211 | 0.0212 | 0.0303 | 0.0164 | 0.0159 | 0.0506 | 0.0649 | 0.02424 | 0.0009 | 0.0113 | |
| 29 | A | 0.1555 | 0.0268 | 0.0338 | 0.0357 | 0.047 | 0.1271 | 0.0165 | 0.023 | 0.0361 | 0.0364 | 0.0072 | 0.0346 | 0.0453 | 0.019 | 0.0148 | 0.0915 | 0.0811 | 0.0533 | 0.0032 | 0.1121 | |
| 30 | V | 0.0757 | 0.0138 | 0.0164 | 0.0212 | 0.0384 | 0.0374 | 0.0098 | 0.1522 | 0.0291 | 0.1328 | 0.0249 | 0.0175 | 0.0225 | 0.0144 | 0.0157 | 0.0367 | 0.0556 | 0.275 | 0.0009 | 0.0148 | |
| 31 | L | 0.0594 | 0.0086 | 0.0147 | 0.0193 | 0.0533 | 0.0285 | 0.0109 | 0.1265 | 0.0313 | 0.2777 | 0.0377 | 0.0174 | 0.021 | 0.017 | 0.0161 | 0.0306 | 0.0432 | 0.1725 | 0.001 | 0.0171 | |
| 32 | D | 0.0759 | 0.0036 | 0.2411 | 0.1604 | 0.0048 | 0.0814 | 0.0307 | 0.0126 | 0.0621 | 0.0142 | 0.0035 | 0.091 | 0.0245 | 0.0503 | 0.0142 | 0.0621 | 0.0418 | 0.0205 | 0.0006 | 0.0061 | |
| 33 | T | 0.1144 | 0.0128 | 0.0884 | 0.0694 | 0.0112 | 0.0734 | 0.0214 | 0.0274 | 0.0661 | 0.0297 | 0.0089 | 0.0581 | 0.0411 | 0.0348 | 0.0215 | 0.114 | 0.1589 | 0.0467 | 0.0019 | 0.0091 | |
| 34 | G | 0.1139 | 0.0052 | 0.0447 | 0.0386 | 0.0099 | 0.4572 | 0.0093 | 0.0095 | 0.031 | 0.0171 | 0.0033 | 0.0389 | 0.0363 | 0.0166 | 0.0092 | 0.0849 | 0.0412 | 0.0298 | 0.0008 | 0.0042 | |
| 35 | I | 0.0629 | 0.0458 | 0.0145 | 0.0194 | 0.0454 | 0.0293 | 0.0099 | 0.1607 | 0.0283 | 0.1721 | 0.0243 | 0.0165 | 0.02 | 0.0141 | 0.016 | 0.0332 | 0.0486 | 0.2149 | 0.0011 | 0.0279 | |
| 36 | S | 0.0793 | 0.0064 | 0.1306 | 0.1238 | 0.018 | 0.0667 | 0.0322 | 0.0192 | 0.0693 | 0.0601 | 0.0111 | 0.0679 | 0.0313 | 0.0588 | 0.0353 | 0.0672 | 0.048 | 0.0325 | 0.0255 | 0.0182 | |
| 37 | T | 0.0961 | 0.0116 | 0.0905 | 0.0759 | 0.0119 | 0.0811 | 0.0351 | 0.0203 | 0.0846 | 0.0431 | 0.0098 | 0.0736 | 0.0479 | 0.0375 | 0.0439 | 0.1036 | 0.0902 | 0.0351 | 0.0027 | 0.0101 | |
| 38 | H | 0.0476 | 0.0098 | 0.0434 | 0.0408 | 0.019 | 0.0339 | 0.3067 | 0.0162 | 0.0502 | 0.0455 | 0.0058 | 0.0685 | 0.0387 | 0.0837 | 0.0518 | 0.0447 | 0.0301 | 0.0319 | 0.0015 | 0.0245 | |
| 39 | P | 0.1048 | 0.0224 | 0.0466 | 0.0594 | 0.009 | 0.0554 | 0.0263 | 0.021 | 0.0583 | 0.037 | 0.0068 | 0.037 | 0.2406 | 0.0448 | 0.0408 | 0.0802 | 0.0597 | 0.0489 | 0.0017 | 0.0105 | |
| 40 | D | 0.0812 | 0.0053 | 0.1846 | 0.1582 | 0.0243 | 0.0885 | 0.0317 | 0.0143 | 0.0598 | 0.02 | 0.0044 | 0.0749 | 0.0289 | 0.0571 | 0.0191 | 0.0663 | 0.0431 | 0.0234 | 0.0013 | 0.0154 | |
| 41 | L | 0.0354 | 0.0044 | 0.0086 | 0.0125 | 0.1952 | 0.0176 | 0.0181 | 0.0651 | 0.0211 | 0.3493 | 0.032 | 0.013 | 0.0174 | 0.0162 | 0.0139 | 0.022 | 0.025 | 0.0735 | 0.0094 | 0.0567 | |
| 42 | N | 0.0574 | 0.0249 | 0.078 | 0.0595 | 0.018 | 0.0543 | 0.0445 | 0.0244 | 0.1485 | 0.0288 | 0.0092 | 0.0579 | 0.0349 | 0.0469 | 0.1542 | 0.0629 | 0.0438 | 0.038 | 0.006 | 0.0097 | |
| 43 | I | 0.1259 | 0.0124 | 0.0358 | 0.0449 | 0.0274 | 0.0672 | 0.0175 | 0.0755 | 0.0439 | 0.082 | 0.0155 | 0.032 | 0.0927 | 0.0291 | 0.0231 | 0.0711 | 0.0638 | 0.1289 | 0.0015 | 0.0159 | |
| 44 | R | 0.0855 | 0.0107 | 0.0501 | 0.0591 | 0.0311 | 0.0792 | 0.0201 | 0.0653 | 0.0899 | 0.0837 | 0.0144 | 0.0383 | 0.0379 | 0.0343 | 0.0477 | 0.0655 | 0.0581 | 0.1033 | 0.0026 | 0.0277 | |
| 45 | G | 0.1156 | 0.008 | 0.0552 | 0.0543 | 0.0283 | 0.1858 | 0.0228 | 0.0194 | 0.0596 | 0.0561 | 0.0113 | 0.0407 | 0.0368 | 0.0387 | 0.0274 | 0.0752 | 0.0583 | 0.0409 | 0.0374 | 0.0306 | |
| 46 | G | 0.1134 | 0.0299 | 0.0419 | 0.0386 | 0.0203 | 0.2158 | 0.0142 | 0.0292 | 0.0485 | 0.048 | 0.0095 | 0.0394 | 0.0421 | 0.0203 | 0.0252 | 0.1032 | 0.0605 | 0.0507 | 0.0263 | 0.025 | |
| 47 | A | 0.0629 | 0.0198 | 0.0262 | 0.026 | 0.1072 | 0.0347 | 0.035 | 0.0346 | 0.0871 | 0.0445 | 0.0089 | 0.0319 | 0.028 | 0.0244 | 0.0656 | 0.0493 | 0.0455 | 0.048 | 0.0357 | 0.1838 | |
| 48 | S | 0.0856 | 0.0153 | 0.1346 | 0.0919 | 0.0307 | 0.0791 | 0.0335 | 0.0193 | 0.0692 | 0.0298 | 0.0063 | 0.0824 | 0.0354 | 0.0365 | 0.0234 | 0.0902 | 0.0724 | 0.0336 | 0.0081 | 0.0262 | |
| 49 | F | 0.0653 | 0.0212 | 0.0216 | 0.0212 | 0.2424 | 0.0395 | 0.0219 | 0.0523 | 0.0297 | 0.1072 | 0.0142 | 0.027 | 0.0231 | 0.0164 | 0.0195 | 0.0443 | 0.043 | 0.0794 | 0.0054 | 0.1124 | |
| 50 | V | 0.1018 | 0.0127 | 004860.0418 0.0270.0722 | 00316 | 00525 | 0.0565 | 0.0621 | 0.0118 | 0.0591 | 0.0482 | 0.0286 | 0.0257 | 0.0844 | 0.0793 | 0.1114 | 0.0264 | 0.0225 | | | | |
| 51 | P | 0.1009 | 0.0095 | 0.1079 | 0.0842 | 0.0169 | 0.1492 | 0.0252 | 0.0203 | 0.0626 | 0.0332 | 0.0066 | 0.0598 | 0.0501 | 0.0348 | 0.0297 | 0.0882 | 0.0652 | 0.0402 | 0.0021 | 0.0168 | |
| 52 | G | 0.0856 | 0.0427 | 0.0757 | 0.0605 | 0.0151 | 0.1636 | 0.0437 | 0.0186 | 0.077 | 0.0269 | 0.0077 | 0.0742 | 0.041 | 0.0359 | 0.0341 | 0.0836 | 0.0562 | 0.035 | 0.0019 | 0.0231 | |
| 53 | E | 0.0902 | 0.0068 | 0.129 | 0.1137 | 0.0119 | 0.1285 | 0.0315 | 0.0216 | 0.0642 | 0.029 | 0.0063 | 0.0663 | 0.0322 | 0.0555 | 0.0249 | 0.0746 | 0.0653 | 0.0337 | 0.0014 | 0.016 | |
| 54 | P | 0.0917 | 0.0155 | 0.0738 | 0.0687 | 0.0291 | 0.1131 | 0.0452 | 0.023 | 0.0591 | 0.0449 | 0.0087 | 0.0568 | 0.0936 | 0.0409 | 0.0321 | 0.0801 | 0.0559 | 0.0384 | 0.0083 | 0.0256 | |
| 55 | S | 0.0865 | 0.01 | 0.1119 | 0.0811 | 0.0446 | 0.0918 | 0.0291 | 0.0297 | 0.0542 | 0.0487 | 0.0078 | 0.0642 | 0.0307 | 0.0342 | 0.0217 | 0.0773 | 0.0613 | 0.0507 | 0.017 | 0.0498 | |
| 56 | T | 0.1068 | 0.0118 | 0.0461 | 0.045 | 0.0347 | 0.0638 | 0.0221 | 0.0297 | 0.0532 | 0.0541 | 0.0082 | 0.0351 | 0.2024 | 0.0323 | 0.0249 | 0.0757 | 0.061 | 0.0527 | 0.002 | 0.048 | |
| 57 | Q | 0.0928 | 0.0112 | 0.0556 | 0.0552 | 0.0203 | 0.0863 | 0.0311 | 0.0336 | 0.0736 | 0.0753 | 0.0179 | 0.0454 | 0.041 | 0.0469 | 0.0548 | 0.0857 | 0.083 | 0.065 | 0.0148 | 0.0143 | |
| 58 | D | 0.0812 | 0.0125 | 0.1662 | 0.1136 | 0.0142 | 0.0822 | 0.0334 | 0.0168 | 0.0684 | 0.0245 | 0.0083 | 0.0834 | 0.032 | 0.0415 | 0.0253 | 0.0759 | 0.0533 | 0.0303 | 0.0252 | 0.0138 | |
| 59 | G | 0.0887 | 0.0378 | 0.0801 | 0.0781 | 0.0447 | 0.1553 | 0.0223 | 0.0195 | 0.0548 | 0.0512 | 0.0073 | 0.0503 | 0.0325 | 0.0359 | 0.0306 | 0.0808 | 0.0533 | 0.0366 | 0.0028 | 0.0395 | |
| 60 | N | 0.0781 | 0.0087 | 0.0913 | 0.0709 | 0.0194 | 0.0836 | 0.0535 | 0.0258 | 0.082 | 0.0578 | 0.0102 | 0.1006 | 0.036 | 0.0466 | 0.0264 | 0.0872 | 0.0617 | 0.0428 | 0.0017 | 0.0183 | |
| 61 | G | 0.0935 | 0.0194 | 0.0428 | 0.0388 | 0.0168 | 0.2763 | 0.022 | 0.0143 | 0.0649 | 0.0235 | 0.0069 | 0.0409 | 0.0383 | 0.0271 | 0.064 | 0.0805 | 0.0448 | 0.03 | 0.0329 | 0.0231 | |
| 62 | H | 0.0365 | 0.0094 | 0.0425 | 0.0407 | 0.0187 | 0.0275 | 0.3485 | 0.01 | 0.0494 | 0.0358 | 0.0045 | 0.0696 | 0.0382 | 0.0924 | 0.0563 | 0.0373 | 0.0241 | 0.024 | 0.0014 | 0.0263 | |
| 63 | G | 0.1131 | 0.005 | 0.0446 | 0.0384 | 0.0099 | 0.47 | 0.0086 | 0.0093 | 0.0302 | 0.0167 | 0.0032 | 0.0382 | 0.0299 | 0.016 | 0.0086 | 0.0846 | 0.0405 | 0.0295 | 0.0008 | 0.0041 | |
| 64 | T | 0.1298 | 0.0141 | 0.0342 | 0.0321 | 0.0132 | 0.063 | 0.0157 | 0.0349 | 0.0683 | 0.0383 | 0.0132 | 0.0443 | 0.0434 | 0.0227 | 0.0215 | 0.1221 | 0.0229 | 0.0625 | 0.0016 | 0.01 | |
| 65 | H | 0.0427 | 0.0097 | 0.0367 | 0.0385 | 0.0218 | 0.0334 | 0.2443 | 0.0143 | 0.0821 | 0.0374 | 0.0064 | 0.056 | 0.0383 | 0.0801 | 0.1236 | 0.0454 | 0.0288 | 0.0263 | 0.0045 | 0.0244 | |
| 66 | V | 0.071 | 0.2457 | 0.0139 | 0.0168 | 0.0129 | 0.0376 | 0.01 | 0.0816 | 0.0223 | 0.0746 | 0.0166 | 0.0154 | 0.0221 | 0.0112 | 0.013 | 0.0484 | 0.0531 | 0.2204 | 0.0007 | 0.015 | |
| 67 | A | 0.2289 | 0.0361 | 0.0414 | 0.0467 | 0.0118 | 0.1126 | 0.0145 | 0.0244 | 0.0387 | 0.0321 | 0.0079 | 0.0362 | 0.0638 | 0.024 | 0.0171 | 0.1078 | 0.0883 | 0.0596 | 0.0015 | 0.0098 | |
| 68 | G | 0.1165 | 0.0068 | 0.0444 | 0.0385 | 0.0103 | 0.4348 | 0.0094 | 0.0103 | 0.033 | 0.0176 | 0.0036 | 0.0395 | 0.0329 | 0.0167 | 0.0104 | 0.0931 | 0.0474 | 0.0306 | 0.0012 | 0.0047 | |
| 69 | T | 0.1029 | 0.0113 | 0.0569 | 0.0803 | 0.0172 | 0.0563 | 0.0174 | 0.0693 | 0.0575 | 0.0617 | 0.0132 | 0.0403 | 0.0387 | 0.0383 | 0.0202 | 0.0859 | 0.1387 | 0.0915 | 0.0013 | 0.0103 | |
| 70 | I | 0.1017 | 0.0126 | 0.0211 | 0.0265 | 0.0296 | 0.0471 | 0.0102 | 0.1548 | 0.0336 | 0.1238 | 0.0246 | 0.0211 | 0.0324 | 0.0167 | 0.0174 | 0.0476 | 0.0607 | 0.2106 | 0.0009 | 0.0121 | |
| 71 | A | 0.197 | 0.0175 | 0.0413 | 0.0439 | 0.012 | 0.1861 | 0.0133 | 0.0234 | 0.0373 | 0.0393 | 0.0078 | 0.0364 | 0.0581 | 0.0223 | 0.0155 | 0.1015 | 0.0787 | 0.0618 | 0.0014 | 0.0084 | |
| 72 | A | 0.1878 | 0.0113 | 0.0425 | 0.0445 | 0.0119 | 0.2181 | 0.0134 | 0.0196 | 0.0398 | 0.0333 | 0.0132 | 0.0377 | 0.0533 | 0.0238 | 0.0161 | 0.1032 | 0.0734 | 0.0502 | 0.0015 | 0.0077 | |
| 73 | L | 0.0876 | 0.0158 | 0.0427 | 0.0447 | 0.022 | 0.0642 | 0.0279 | 0.0447 | 0.1121 | 0.1044 | 0.0171 | 0.0473 | 0.0345 | 0.038 | 0.0559 | 0.0708 | 0.0712 | 0.0868 | 0.0025 | 0.0151 | |
| 74 | N | 0.0863 | 0.0071 | 0.1046 | 0.0784 | 0.0171 | 0.1274 | 0.0466 | 0.0178 | 0.0797 | 0.0304 | 0.0106 | 0.0989 | 0.0495 | 0.0379 | 0.0233 | 0.0844 | 0.0568 | 0.0322 | 0.0013 | 0.013 | |
| 75 | N | 0.0772 | 0.0084 | 0.1015 | 0.0709 | 0.0187 | 0.0786 | 0.0472 | 0.0208 | 0.0863 | 0.0451 | 0.0073 | 0.1178 | 0.0306 | 0.0354 | 0.024 | 0.0897 | 0.0668 | 0.0351 | 0.0099 | 0.031 | |
| 76 | S | 0.1123 | 0.0112 | 0.0671 | 0.0563 | 0.017 | 0.2039 | 0.031 | 0.016 | 0.0598 | 0.024 | 0.0058 | 0.0651 | 0.0481 | 0.029 | 0.0277 | 0.1078 | 0.0751 | 0.0333 | 0.0023 | 0.0105 | |
| 77 | I | 0.0771 | 0.0116 | 0.0371 | 0.0401 | 0.0724 | 0.0624 | 0.024 | 0.076 | 0.064 | 0.0823 | 0.0142 | 0.0363 | 0.0364 | 0.0269 | 0.043 | 0.0597 | 0.0681 | 0.1099 | 0.0033 | 0.06 | |
| 78 | G | 0.0866 | 0.2259 | 0.0354 | 0.0326 | 0.0092 | 0.2594 | 0.0121 | 0.0134 | 0.0427 | 0.0214 | 0.0043 | 0.032 | 0.0277 | 0.0179 | 0.0183 | 0.0794 | 0.0407 | 0.0301 | 0.0012 | 0.0113 | |
| 79 | V | 0.099 | 0.0124 | 0.0272 | 0.029 | 0.0313 | 0.1352 | 0.0171 | 0.0737 | 0.0424 | 0.0805 | 0.0252 | 0.0285 | 0.031 | 0.0208 | 0.0243 | 0.0667 | 0.0735 | 0.1494 | 0.0018 | 0.0338 | |
| 80 | L | 0.0823 | 0.0146 | 0.0311 | 0.0345 | 0.0584 | 0.052 | 0.0277 | 0.0591 | 0.0469 | 0.1066 | 0.0145 | 0.0301 | 0.0286 | 0.0256 | 0.0336 | 0.0537 | 0.0544 | 0.1227 | 0.0036 | 0.1194 | |
| 81 | G | 0.1143 | 0.0051 | 0.0445 | 0.0384 | 0.0099 | 0.4638 | 0.0087 | 0.0096 | 0.0306 | 0.017 | 0.0033 | 0.0382 | 0.0303 | 0.0161 | 0.0088 | 0.085 | 0.0425 | 0.03 | 0.0008 | 0.0042 | |
| 82 | V | 0.0835 | 0.0138 | 0.0176 | 0.0222 | 0.0304 | 0.0486 | 0.0101 | 0.1244 | 0.0301 | 0.137 | 0.0287 | 0.0182 | 0.0247 | 0.0152 | 0.0152 | 0.0407 | 0.0583 | 0.2712 | 0.0008 | 0.0129 | |
| 83 | A | 0.2202 | 0.0127 | 0.0437 | 0.0476 | 0.0133 | 0.1093 | 0.0158 | 0.0257 | 0.0416 | 0.0551 | 0.011 | 0.0384 | 0.0617 | 0.0251 | 0.0199 | 0.1023 | 0.0843 | 0.064 | 0.0015 | 0.0095 | |
| 84 | P | 0.075 | 0.0132 | 0.0214 | 0.0253 | 0.0857 | 0.0373 | 0.026 | 0.0179 | 0.0877 | 0.0362 | 0.0069 | 0.0292 | 0.1918 | 0.0282 | 0.0642 | 0.063 | 0.0442 | 0.0294 | 0.0049 | 0.1204 | |
| 85 | S | 0.0786 | 0.007 | 0.088 | 0.0719 | 0.014 | 0.1453 | 0.0382 | 0.0153 | 0.131 | 0.0276 | 0.0076 | 0.0793 | 0.0318 | 0.0396 | 0.0568 | 0.081 | 0.0527 | 0.0261 | 0.0024 | 0.0099 | |
| 86 | A | 0.1928 | 0.0253 | 0.0383 | 0.0437 | 0.0131 | 0.0976 | 0.0173 | 0.04 | 0.0403 | 0.0483 | 0.0106 | 0.035 | 0.0556 | 0.0252 | 0.0179 | 0.0991 | 0.0938 | 0.0987 | 0.0015 | 0.0099 | |
| 87 | E | 0.0688 | 0.0088 | 0.0701 | 0.0703 | 0.009 | 0.0626 | 0.0432 | 0.0162 | 0.1658 | 0.0261 | 0.0096 | 0.0616 | 0.037 | 0.0649 | 0.109 | 0.0793 | 0.058 | 0.0259 | 0.0103 | 0.0076 | |
| 88 | L | 0.0551 | 0.0084 | 0.0127 | 0.0182 | 0.0401 | 0.0253 | 0.0098 | 0.1377 | 0.028 | 0.3051 | 0.032 | 0.0149 | 0.0201 | 0.0167 | 0.016 | 0.0278 | 0.042 | 0.1739 | 0.0067 | 0.0136 | |
| 89 | Y | 0.0869 | 0.0108 | 0.025 | 0.0267 | 0.0709 | 0.1069 | 0.0259 | 0.0596 | 0.0324 | 0.143 | 0.0232 | 0.0279 | 0.0277 | 0.0208 | 0.0161 | 0.0515 | 0.0458 | 0.0981 | 0.0144 | 0.0871 | |
| 90 | A | 0.1408 | 0.0112 | 0.041 | 0.0421 | 0.0229 | 0.2166 | 0.0138 | 0.0341 | 0.0376 | 0.0463 | 0.0097 | 0.0364 | 0.0749 | 0.0221 | 0.0164 | 0.0925 | 0.0619 | 0.0683 | 0.0017 | 0.0137 | |
| 91 | V | 0.0739 | 0.015 | 0.016 | 0.0203 | 0.0602 | 0.0433 | 0.0123 | 0.1147 | 0.0276 | 0.1188 | 0.0259 | 0.0181 | 0.0217 | 0.0137 | 0.014 | 0.0361 | 0.0502 | 0.2368 | 0.002 | 0.0808 | |
| 92 | K | 0.0427 | 0.0074 | 0.0344 | 0.0341 | 0.0079 | 0.0389 | 0.0359 | 0.0162 | 0.2636 | 0.0239 | 0.0125 | 0.0422 | 0.0329 | 0.0454 | 0.02303 | 0.0579 | 0.0432 | 0.0225 | 0.0082 | 0.0044 | |
| 93 | V | 0.0893 | 0.0126 | 0.02 | 0.0246 | 0.0249 | 0.0459 | 0.0118 | 0.0994 | 0.0449 | 0.1359 | 0.0564 | 0.0212 | 0.0272 | 0.0195 | 0.0187 | 0.0449 | 0.0565 | 0.2364 | 0.0009 | 0.0111 | |
| 94 | L | 0.0464 | 0.0273 | 0.015 | 0.0184 | 0.1074 | 0.0451 | 0.0126 | 0.0483 | 0.0235 | 0.3892 | 0.0306 | 0.0164 | 0.021 | 0.0182 | 0.0133 | 0.0324 | 0.031 | 0.0686 | 0.009 | 0.031 | |
| 95 | G | 0.0893 | 0.009 | 0.1232 | 0.0891 | 0.0141 | 0.1431 | 0.0277 | 0.0175 | 0.0629 | 0.0267 | 0.0056 | 0.069 | 0.036 | 0.0398 | 0.0356 | 0.0863 | 0.0533 | 0.0329 | 0.0203 | 0.0203 | |
| 96 | A | 0.0971 | 0.042 | 0.0775 | 0.0719 | 0.0165 | 0.1056 | 0.033 | 0.0184 | 0.0861 | 0.0435 | 0.0081 | 0.0572 | 0.0554 | 0.0492 | 0.0498 | 0.0864 | 0.0568 | 0.0358 | 0.0026 | 0.0105 | |
| 97 | S | 0.0941 | 0.0115 | 0.0798 | 0.0628 | 0.0357 | 0.164 | 0.0306 | 0.0186 | 0.0682 | 0.0274 | 0.0062 | 0.0728 | 0.0373 | 0.039 | 0.0272 | 0.1038 | 0.0625 | 0.0307 | 0.0028 | 0.0274 | |
| 98 | G | 0.1032 | 0.0361 | 0.0525 | 0.0536 | 0.0167 | 0.3154 | 0.0166 | 0.0163 | 0.0443 | 0.0361 | 0.0072 | 0.0404 | 0.0438 | 0.0259 | 0.0173 | 0.0813 | 0.0466 | 0.0359 | 0.0013 | 0.012 | |
| 99 | S | 0.1025 | 0.0146 | 0.0577 | 0.058 | 0.0417 | 0.1311 | 0.0229 | 0.0262 | 0.0636 | 0.0494 | 0.0133 | 0.046 | 0.0511 | 0.0363 | 0.0288 | 0.1112 | 0.0651 | 0.0455 | 0.0153 | 0.0233 | |
| 100 | G | 0.1015 | 0.0322 | 0.0389 | 0.039 | 0.0191 | 0.2539 | 0.0124 | 0.0429 | 0.0392 | 0.0668 | 0.0163 | 0.0342 | 0.0316 | 0.0211 | 0.0197 | 0.0784 | 0.055 | 0.09 | 0.0015 | 0.0089 | |
| 101 | S | 0.1062 | 0.016 | 0.0489 | 0.0485 | 0.0416 | 0.0731 | 0.023 | 0.0244 | 0.0636 | 0.0405 | 0.0089 | 0.0469 | 0.0498 | 0.0348 | 0.0261 | 0.109 | 0.1235 | 0.044 | 0.0035 | 0.0732 | |
| 102 | V | 0.0887 | 0.0103 | 0.0838 | 0.0641 | 0.0322 | 0.078 | 0.024 | 0.033 | 0.0548 | 0.0907 | 0.0251 | 0.0493 | 0.0366 | 0.0307 | 0.0236 | 0.0725 | 0.08 | 0.0583 | 0.0081 | 0.0585 | |
| 103 | S | 0.1296 | 0.0165 | 0.0581 | 0.0627 | 0.0161 | 0.0896 | 0.0196 | 0.0326 | 0.0642 | 0.0554 | 0.0116 | 0.0467 | 0.0494 | 0.035 | 0.0309 | 0.1212 | 0.0846 | 0.0573 | 0.0091 | 0.0137 | |
| 104 | S | 0.0891 | 0.0093 | 0.0895 | 0.0681 | 0.0158 | 0.1081 | 0.0198 | 0.053 | 0.0458 | 0.0617 | 0.0121 | 0.05 | 0.0294 | 0.0304 | 0.0205 | 0.0695 | 0.0565 | 0.0981 | 0.0664 | 0.0098 | |
| 105 | I | 0.0699 | 0.01 | 0.0483 | 0.0749 | 0.0345 | 0.0426 | 0.0142 | 0.1278 | 0.0393 | 0.1167 | 0.0232 | 0.0274 | 0.0231 | 0.0319 | 0.018 | 0.0397 | 0.0498 | 0.169 | 0.0188 | 0.025 | |
| 106 | A | 0.141 | 0.0112 | 0.0301 | 0.0346 | 0.0259 | 0.071 | 0.0143 | 0.0834 | 0.0409 | 0.1438 | 0.0181 | 0.0298 | 0.0419 | 0.0228 | 0.0205 | 0.0693 | 0.0697 | 0.1188 | 0.0013 | 0.0154 | |
| 107 | Q | 0.1125 | 0.016 | 0.0808 | 0.0824 | 0.0112 | 0.0815 | 0.0335 | 0.0184 | 0.0952 | 0.0357 | 0.0085 | 0.0559 | 0.0444 | 0.0655 | 0.0592 | 0.0885 | 0.0615 | 0.0368 | 0.0029 | 0.0123 | |
| 108 | G | 0.1541 | 0.0111 | 0.0432 | 0.0411 | 0.0116 | 0.2955 | 0.012 | 0.0155 | 0.0384 | 0.0284 | 0.0073 | 0.0402 | 0.046 | 0.0199 | 0.0149 | 0.1082 | 0.0654 | 0.0407 | 0.0017 | 0.0068 | |
| 109 | L | 0.0497 | 0.0082 | 0.0123 | 0.0168 | 0.1072 | 0.0262 | 0.0112 | 0.1252 | 0.037 | 0.2201 | 0.0518 | 0.0157 | 0.0182 | 0.0156 | 0.0209 | 0.0291 | 0.0397 | 0.1401 | 0.0141 | 0.0454 | |
| 110 | E | 0.0804 | 0.014 | 0.0988 | 0.098 | 0.0171 | 0.1086 | 0.0309 | 0.0344 | 0.0784 | 0.0345 | 0.0094 | 0.0634 | 0.032 | 0.0524 | 0.0465 | 0.0735 | 0.0542 | 0.0475 | 0.0024 | 0.0252 | |
| 111 | W | 0.0369 | 0.0085 | 0.0293 | 0.0291 | 0.1137 | 0.0234 | 0.0437 | 0.0241 | 0.0429 | 0.0832 | 0.0114 | 0.0261 | 0.0234 | 0.0256 | 0.045 | 0.0363 | 0.0252 | 0.0291 | 0.2235 | 0.1198 | |
| 112 | A | 0.1607 | 0.0287 | 0.0453 | 0.046 | 0.0141 | 0.0974 | 0.0236 | 0.0374 | 0.0515 | 0.066 | 0.0156 | 0.0469 | 0.0483 | 0.0279 | 0.0294 | 0.0873 | 0.0735 | 0.0904 | 0.0017 | 0.0106 | |
| 113 | G | 0.1274 | 0.0124 | 0.0278 | 0.0329 | 0.0278 | 0.0747 | 0.0159 | 0.076 | 0.0472 | 0.0906 | 0.0193 | 0.0281 | 0.0783 | 0.0255 | 0.0307 | 0.0727 | 0.0749 | 0.1185 | 0.0019 | 0.023 | |
| 114 | N | 0.0898 | 0.0079 | 0.1008 | 0.0945 | 0.0096 | 0.0889 | 0.0465 | 0.0182 | 0.0868 | 0.0315 | 0.0074 | 0.0728 | 0.0386 | 0.077 | 0.0501 | 0.0803 | 0.0596 | 0.0308 | 0.0022 | 0.0091 | |
| 115 | N | 0.081 | 0.0068 | 0.1106 | 0.0853 | 0.0121 | 0.0882 | 0.0637 | 0.0185 | 0.0805 | 0.0477 | 0.0074 | 0.0822 | 0.0371 | 0.0568 | 0.0356 | 0.0731 | 0.0491 | 0.0301 | 0.0245 | 0.0114 | |
| 116 | G | 0.0798 | 0.0082 | 0.0484 | 0.0433 | 0.016 | 0.1798 | 0.0705 | 0.0184 | 0.0958 | 0.0362 | 0.0075 | 0.0552 | 0.0511 | 0.0421 | 0.0674 | 0.0718 | 0.048 | 0.0374 | 0.0029 | 0.0219 | |
| 117 | M | 0.1101 | 0.0161 | 0.0305 | 0.0325 | 0.0321 | 0.0889 | 0.0166 | 0.0806 | 0.0686 | 0.0865 | 0.0337 | 0.0332 | 0.0492 | 0.024 | 0.0363 | 0.0625 | 0.0595 | 0.1291 | 0.0017 | 0.0127 | |
| 118 | H | 0.0825 | 0.0141 | 0.1297 | 0.0962 | 0.0134 | 0.0707 | 0.0601 | 0.0215 | 0.0817 | 0.0318 | 0.0072 | 0.0656 | 0.0368 | 0.051 | 0.0597 | 0.0705 | 0.0493 | 0.0469 | 0.0026 | 0.01 | |
| 119 | V | 0.0869 | 0.0136 | 0.0195 | 0.0254 | 0.0271 | 0.0415 | 0.0099 | 0.1578 | 0.0309 | 0.1215 | 0.0226 | 0.0193 | 0.0252 | 0.0157 | 0.0162 | 0.0408 | 0.0602 | 0.2584 | 0.0007 | 0.0119 | |
| 120 | A | 0.0801 | 0.0142 | 0.0186 | 0.023 | 0.0686 | 0.0392 | 0.0193 | 0.1298 | 0.0303 | 0.1205 | 0.0194 | 0.0221 | 0.024 | 0.0159 | 0.0159 | 0.0409 | 0.0509 | 0.1378 | 0.0028 | 0.1277 | |
| 121 | N | 0.0975 | 0.0212 | 0.0747 | 0.0516 | 0.0136 | 0.093 | 0.0395 | 0.021 | 0.084 | 0.0268 | 0.0069 | 0.1031 | 0.0433 | 0.0298 | 0.0306 | 0.1348 | 0.0829 | 0.0323 | 0.0032 | 0.0134 | |
| 122 | L | 0.0851 | 0.0405 | 0.0279 | 0.0282 | 0.0365 | 0.0441 | 0.0149 0.0563 | 0.0644 | 0.1899 | 0.1067 | 0.0302 | 0.0281 | 0.023 | 0.023 | 0.05 | 0.0485 | 0.0845 | 0.0012 | 0.0169 | | |
| 123 | S | 0.127 | 0.0324 | 0.0423 | 0.0374 | 0.0141 | 0.1074 | 0.0181 | 0.0169 | 0.0617 | 0.023 | 0.0073 | 0.0555 | 0.0634 | 0.0226 | 0.0306 | 0.1909 | 0.1035 | 0.0342 | 0.0054 | 0.0104 | |
| 124 | L | 0.0335 | 0.0039 | 0.009 | 0.0125 | 0.0542 | 0.0203 | 0.0125 | 0.0455 | 0.0217 | 0.321 | 0.0257 | 0.013 | 0.0158 | 0.0164 | 0.0263 | 0.0257 | 0.0222 | 0.0644 | 0.2266 | 0.0309 | |
| 125 | G | 0.1119 | 0.0057 | 0.046 | 0.0392 | 0.0114 | 0.4475 | 0.0096 | 0.0106 | 0.0317 | 0.0179 | 0.0035 | 0.0395 | 0.0302 | 0.0165 | 0.0093 | 0.0857 | 0.0433 | 0.0322 | 0.001 | 0.0086 | |
| 126 | S | 0.1107 | 0.0165 | 0.0423 | 0.0424 | 0.0158 | 0.2418 | 0.0167 | 0.0205 | 0.0408 | 0.0279 | 0.0055 | 0.0396 | 0.129 | 0.0261 | 0.0189 | 0.0966 | 0.059 | 0.0432 | 0.0017 | 0.0113 | |
| 127 | P | 0.1071 | 0.0108 | 0.0669 | 0.0602 | 0.0227 | 0.1725 | 0.0231 | 0.0223 | 0.0539 | 0.0292 | 0.0064 | 0.0492 | 0.0931 | 0.0301 | 0.0277 | 0.0921 | 0.0687 | 0.0453 | 0.0022 | 0.0217 | |
| 128 | S | 0.0907 | 0.0176 | 0.086 | 0.0693 | 0.0302 | 0.1111 | 0.0234 | 0.028 | 0.0696 | 0.048 | 0.0099 | 0.0517 | 0.0396 | 0.0352 | 0.0347 | 0.0831 | 0.0615 | 0.0474 | 0.003 | 0.0616 | |
| 129 | P | 0.0957 | 0.0116 | 0.1026 | 0.0796 | 0.0226 | 0.1378 | 0.0307 | 0.0241 | 0.0533 | 0.0376 | 0.0083 | 0.0613 | 0.0541 | 0.0354 | 0.0207 | 0.0974 | 0.0593 | 0.0436 | 0.0082 | 0.0189 | |
| 130 | S | 0.1101 | 0.0143 | 0.0576 | 0.0547 | 0.0159 | 0.1545 | 0.0302 | 0.0232 | 0.0597 | 0.0378 | 0.0116 | 0. 0498 | 0.0537 | 0.0303 | 0.0422 | 0.1092 | 0.0715 | 0.0537 | 0.0033 | 0.0192 | |
| 131 | A | 0.0944 | 0.0111 | 0.0708 | 0.0689 | 0.0201 | 0.0693 | 0.0331 | 0.0216 | 0.095 | 0.0423 | 0.009 | 0.0498 | 0.0742 | 0.0482 | 0.0614 | 0.0851 | 0.0727 | 0.0434 | 0.0091 | 0.0255 | |
| 132 | T | 0.1235 | 0.0103 | 0.0384 | 0.0455 | 0.024 | 0.0735 | 0.0165 | 0.0434 | 0.0529 | 0.1424 | 0.0204 | 0.0346 | 0.067 | 0.0271 | 0.0256 | 0.0792 | 0.0869 | 0.0759 | 0.0075 | 0.0111 | |
| 133 | L | 0.0929 | 0.0143 | 0.0272 | 0.0364 | 0.0659 | 0.0591 | 0.0141 | 0.0653 | 0.0358 | 0.206 | 0.0282 | 0.0238 | 0.0337 | 0.0252 | 0.0165 | 0.0532 | 0.0653 | 0.1053 | 0.0076 | 0.0288 | |
| 134 | E | 0.0776 | 0.0079 | 0.0721 | 0.0822 | 0.0137 | 0.0597 | 0.0515 | 0.0206 | 0.122 | 0.0505 | 0.0147 | 0.0604 | 0.0369 | 0.0734 | 0.0782 | 0.0691 | 0.0552 | 0.0367 | 0.0031 | 0.0173 | |
| 135 | Q | 0.0783 | 0.0081 | 0.0744 | 0.0789 | 0.022 | 0.0563 | 0.0381 | 0.0366 | 0.1043 | 0.0714 | 0.0167 | 0.0548 | 0.0358 | 0.0708 | 0.0617 | 0.0691 | 0.0595 | 0.0536 | 0.0026 | 0.0109 | |
| 136 | A | 0.2134 | 0.0114 | 0.0437 | 0.0485 | 0.0148 | 0.126 | 0.0143 | 0.0316 | 0.04 | 0.0501 | 0.0155 | 0.0346 | 0.058 | 0.0248 | 0.0188 | 0.0925 | 0.0805 | 0.0699 | 0.0013 | 0.0131 | |
| 137 | V | 0.0868 | 0.0231 | 0.0216 | 0.0241 | 0.0968 | 0.046 | 0.0133 | 0.0921 | 0.035 | 0.1328 | 0.0278 | 0.023 | 0.0282 | 0.0166 | 0.022 | 0.0513 | 0.0534 | 0.1728 | 0.0084 | 0.0298 | |
| 138 | N | 0.0811 | 0.0079 | 0.0904 | 0.0852 | 0.0507 | 0.0598 | 0.0317 | 0.0389 | 0.0885 | 0.0529 | 0.0142 | 0.0645 | 0.0306 | 0.0448 | 0.046 | 0.0632 | 0.0487 | 0.0531 | 0.0087 | 0.0417 | |
| 139 | S | 0.073 | 0.0109 | 0.0554 | 0.0552 | 0.0448 | 0.0564 | 0.0466 | 0.0196 | 0.0995 | 0.0578 | 0.009 | 0.0539 | 0.0322 | 0.0429 | 0.0904 | 0.0626 | 0.0454 | 0.0331 | 0.017 | 0.0932 | |
| 140 | A | 0.1788 | 0.0101 | 0.0373 | 0.0404 | 0.0267 | 0.1774 | 0.0128 0.0303 | 0.0342 | 0.0807 | 0.0119 | 0.0326 | 0.0494 | 0.0213 | 0.0146 | 0.0862 | 0.069 | 0.0626 | 0.0073 | 0.0191 | | |
| 141 | T | 0.0753 | 0.0134 | 0.0249 | 0.0294 | 0.0688 | 0.0394 | 0.0194 | 0.0788 | 0.0539 | 0.0836 | 0.0196 | 0.0289 | 0.026 | 0.0254 | 0.0338 | 0.0525 | 0.0727 | 0.1395 | 0.0269 | 0.0909 | |
| 142 | S | 0.0964 | 0.0093 | 0.0805 | 0.0851 | 0.0119 | 0.0719 | 0.0376 | 0.0205 | 0.1105 | 0.0359 | 0.0087 | 0.0671 | 0.0396 | 0.0566 | 0.0652 | 0.0855 | 0.0709 | 0.0343 | 0.003 | 0.0134 | |
| 143 | R | 0.09 | 0.0108 | 0.0548 | 0.0672 | 0.0126 | 0.062 | 0.0426 | 0.0189 | 0.1231 | 0.0431 | 0.0128 | 0.0515 | 0.0483 | 0.061 | 0.1064 | 0.0841 | 0.0554 | 0.0347 | 0.0106 | 0.0125 | |
| 144 | G | 0.1083 | 0.0052 | 0.0547 | 0.0456 | 0.01 | 0.4187 | 0.0163 | 0.0102 | 0.0374 | 0.0179 | 0.0035 | 0.0479 | 0.0302 | 0.0214 | 0.011 | 0.0842 | 0.0436 | 0.0291 | 0.0008 | 0.0053 | |
| 145 | V | 0.1031 | 0.0219 | 0.0271 | 0.0286 | 0.0291 | 0.0612 | 0.0138 | 0.0991 | 0.0445 | 0.0937 | 0.019 | 0.0317 | 0.0357 | 0.0183 | 0.0228 | 0.0808 | 0.0745 | 0.1846 | 0.0021 | 0.0132 | |
| 146 | L | 0.0653 | 0.01 | 0.0162 | 0.0203 | 0.0581 | 0.033 | 0.0191 | 0.1128 | 0.0319 | 0.247 | 0.0291 | 0.0199 | 0.0241 | 0.0185 | 0.0172 | 0.0418 | 0.057 | 0.1629 | 0.0014 | 0.0192 | |
| 147 | V | 0.0633 | 0.0123 | 0.0167 | 0.0193 | 0.1581 | 0.0347 | 0.0151 | 0.0896 | 0.0283 | 0.1304 | 0.0205 | 0.0214 | 0.0252 | 0.0153 | 0.0155 | 0.044 | 0.0492 | 0.1502 | 0.0097 | 0.0866 | |
| 148 | V | 0.1037 | 0.0257 | 0.0217 | 0.0272 | 0.0196 | 0.0526 | 0.0112 | 0.1077 | 0.0316 | 0.1194 | 0.0234 | 0.0208 | 0.0306 | 0.0171 | 0.0181 | 0.051 | 0.0607 | 0.2493 | 0.0009 | 0.0108 | |
| 149 | A | 0.1019 | 0.0555 | 0.0215 | 0.0244 | 0.0726 | 0.0577 | 0.0138 | 0.0557 | 0.0305 | 0.0658 | 0.0111 | 0.0228 | 0.0312 | 0.0168 | 0.0288 | 0.0601 | 0.0534 | 0.1096 | 0.1445 | 0.0264 | |
| 150 | A | 0.2296 | 0.0148 | 0.0429 | 0.0476 | 0.012 | 0.1135 | 0.015 | 0.0223 | 0.0414 | 0.0314 | 0.0079 | 0.0388 | 0.0659 | 0.0247 | 0.0185 | 0.1172 | 0.091 | 0.0575 | 0.0019 | 0.0094 | |
| 151 | S | 0.2085 | 0.0161 | 0.0423 | 0.0454 | 0.0131 | 0.1091 | 0.0156 | 0.0264 | 0.0446 | 0.0368 | 0.0085 | 0.0412 | 0.0632 | 0.0242 | 0.0201 | 0.124 | 0.0932 | 0.0592 | 0.0023 | 0.0097 | |
| 152 | G | 0.1126 | 0.005 | 0.0443 | 0.0382 | 0.0101 | 0.4664 | 0.0086 | 0.0097 | 0.0307 | 0.0184 | 0.0049 | 0.038 | 0.0298 | 0.0161 | 0.0088 | 0.0842 | 0.0405 | 0.03 | 0.0008 | 0.0041 | |
| 153 | N | 0.075 | 0.0069 | 0.1101 | 0.0687 | 0.0121 | 0.0801 | 0.0583 | 0.0171 | 0.1024 | 0.0263 | 0.0058 | 0.1469 | 0.0305 | 0.037 | 0.0264 | 0.0942 | 0.063 | 0.0247 | 0.0014 | 0.0158 | |
| 154 | S | 0.0982 | 0.0084 | 0.1155 | 0.1144 | 0.0157 | 0.1807 | 0.0265 | 0.014 | 0.0546 | 0.0204 | 0.0047 | 0.064 | 0.0345 | 0.0445 | 0.0173 | 0.0881 | 0.0541 | 0.0278 | 0.0017 | 0.0169 | |
| 155 | G | 0.105 | 0.0068 | 0.0632 | 0.0502 | 0.0136 | 0.3316 | 0.0213 | 0.0132 | 0.0485 | 0.0248 | 0.0045 | 0.0638 | 0.0316 | 0.0228 | 0.0139 | 0.0888 | 0.0488 | 0.0319 | 0.0013 | 0.0156 | |
| 156 | A | 0.0941 | 0.0118 | 0.0543 | 0.0556 | 0.0143 | 0.0713 | 0.0333 | 0.026 | 0.0807 | 0.0651 | 0.0116 | 0.0457 | 0.0947 | 0.0514 | 0.0828 | 0.0881 | 0.0626 | 0.0444 | 0.004 | 0.0122 | |
| 157 | G | 0.0889 | 0.0185 | 0.0666 | 0.0639 | 0.0501 | 0.1171 | 0.0362 | 0.0215 | 0.0623 | 0.0464 | 0.0077 | 0.0567 | 0.04 | 0.0457 | 0.042 | 0.0869 | 0.0598 | 0.0364 | 0.035 | 0.0517 | |
| 158 | S | 0.1188 | 0.025 | 0.0466 | 0.0533 | 0.0333 | 0.0836 | 0.0189 | 0.0343 | 0.0663 | 0.0405 | 0.0125 | 0.0462 | 0.0589 | 0.0278 | 0.0237 | 0.1092 | 0.1159 | 0.0615 | 0.0026 | 0.0286 | |
| 159 | I | 0.0626 | 0.1691 | 0.022 | 0.0222 | 0.0669 | 0.053 | 0.0231 | 0.0642 | 0.0341 | 0.0964 | 0.017 | 0.0258 | 0.031 | 0.0188 | 0.0263 | 0.0529 | 0.0423 | 0.1124 | 0.0026 | 0.0597 | |
| 160 | S | 0.0979 | 0.011 | 0.0942 | 0.0687 | 0.0394 | 0.1415 | 0.0275 | 0.0218 | 0.0517 | 0.0413 | 0.0084 | 0.0586 | 0.0334 | 0.029 | 0.0197 | 0.0868 | 0.0714 | 0.04 | 0.0026 | 0.0597 | |
| 161 | Y | 0.0902 | 0.0221 | 0.0325 | 0.0305 | 0.0685 | 0.1531 | 0.0182 | 0.0271 | 0.038 | 0.048 | 0.0089 | 0.0384 | 0.0381 | 0.017 | 0.0166 | 0.0897 | 0.0614 | 0.0492 | 0.0161 | 0.1358 | |
| 162 | P | 0.0899 | 0.0154 | 0.0211 | 0.0278 | 0.0507 | 0.0445 | 0.0277 | 0.0133 | 0.0336 | 0.0356 | 0.0046 | 0.0255 | 0.2818 | 0.0314 | 0.0241 | 0.0697 | 0.0432 | 0.0316 | 0.0032 | 0.1334 | |
| 163 | A | 0.1843 | 0.0115 | 0.0501 | 0.0494 | 0.0114 | 0.1513 | 0.0172 | 0.0224 | 0.0507 | 0.0301 | 0.0078 | 0.0454 | 0.056 | 0.0265 | 0.018 | 0.1043 | 0.1067 | 0.0521 | 0.0014 | 0.0088 | |
| 164 | R | 0.1057 | 0.019 | 0.0567 | 0.0475 | 0.0198 | 0.0904 | 0.0295 | 0.0269 | 0.0958 | 0.0415 | 0.0109 | 0.0629 | 0.0424 | 0.0333 | 0.0672 | 0.1039 | 0.0762 | 0.0504 | 0.0039 | 0.0192 | |
| 165 | Y | 0.1069 | 0.0465 | 0.0363 | 0.0435 | 0.0652 | 0.0694 | 0.0195 | 0.0312 | 0.0388 | 0.0643 | 0.009 | 0.0362 | 0.0386 | 0.0235 | 0.0178 | 0.0932 | 0.0651 | 0.0535 | 0.0044 | 0.1363 | |
| 166 | A | 0.098 | 0.011 | 0.058 | 0.0604 | 0.0205 | 0.0616 | 0.0224 | 0.0623 | 0.0641 | 0.061 | 0.0187 | 0.0436 | 0.1247 | 0.0339 | 0.0405 | 0.0777 | 0.0624 | 0.0748 | 0.0021 | 0.0098 | |
| 167 | N | 0.0713 | 0.0172 | 0.0604 | 0.0553 | 0.0571 | 0.08 | 0.0307 | 0.0164 | 0.0727 | 0.0384 | 0.0065 | 0.0605 | 0.0295 | 0.0295 | 0.0339 | 0.0816 | 0.0531 | 0.0259 | 0.0984 | 0.0828 | |
| 168 | A | 0.1301 | 0.0201 | 0.0281 | 0.0307 | 0.018 | 0.0685 | 0.013 | 0.0748 | 0.0399 | 0.0809 | 0.0157 | 0.03 | 0.039 | 0.0185 | 0.0176 | 0.0784 | 0.1025 | 0.1719 | 0.0131 | 0.0147 | |
| 169 | M | 0.057 | 0.0486 | 0.0136 | 0.0185 | 0.0541 | 0.0257 | 0.0097 | 0.1495 | 0.0343 | 0.2217 | 0.0411 | 0.0163 | 0.0199 | 0.0157 | 0.0176 | 0.0333 | 0.0476 | 0.1619 | 0.0011 | 0.018 | |
| 170 | A | 0.1527 | 0.0169 | 0.0397 | 0.039 | 0.0131 | 0.0999 | 0.017 | 0.0278 | 0.0613 | 0.0325 | 0.0095 | 0.0461 | 0.0528 | 0.0259 | 0.0235 | 0.1341 | 0.1493 | 0.0545 | 0.0026 | 0.0097 | |
| 171 | V | 0.0855 | 0.015 | 0.0174 | 0.0224 | 0.0216 | 0.0426 | 0.0098 | 0.1419 | 0.0278 | 0.1144 | 0.0226 | 0.0177 | 0.0243 | 0.0145 | 0.0149 | 0.0384 | 0.0581 | 0.3032 | 0.0006 | 0.0111 | |
| 172 | G | 0.1466 | 0.0108 | 0.0453 | 0.0415 | 0.0112 | 0.3 | 0.0136 | 0.0153 | 0.0413 | 0.023 | 0.0054 | 0.0439 | 0.0443 | 0.0203 | 0.0177 | 0.1075 | 0.0651 | 0.0407 | 0.0018 | 0.0069 | |
| 173 | A | 0.2022 | 0.0225 | 0.0447 | 0.0461 | 0.0123 | 0.12 | 0.0159 | 0.0207 | 0.0457 | 0.0289 | 0.0076 | 0.0434 | 0.0631 | 0.0242 | 0.0207 | 0.1308 | 0.0909 | 0.0513 | 0.0026 | 0.0096 | |
| 174 | T | 0.1181 | 0.0196 | 0.0288 | 0.0314 | 0.0338 | 0.0741 | 0.0162 | 0.0728 | 0.0427 | 0.098 | 0.0156 | 0.0318 | 0.038 | 0.0197 | 0.0181 | 0.0835 | 0.107 | 0.1253 | 0.0019 | 0.0296 | |
| 175 | D | 0.1006 | 0.01 | 0.1135 | 0.0881 | 0.011 | 0.0872 | 0.0278 | 0.0295 | 0.0693 | 0.0346 | 0.0082 | 0.0686 | 0.0366 | 0.0435 | 0.0268 | 0.0931 | 0.0979 | 0.0484 | 0.0017 | 0.0089 | |
| 176 | Q | 0.0839 | 0.0111 | 0.0607 | 0.072 | 0.0365 | 0.0642 | 0.0343 | 0.0343 | 0.0866 | 0.0694 | 0.0129 | 0.0489 | 0.0479 | 0.0647 | 0.0501 | 0.0849 | 0.0623 | 0.0518 | 0.0091 | 0.0185 | |
| 177 | N | 0.0986 | 0.011 | 0.0856 | 0.0798 | 0.0292 | 0.0846 | 0.0356 | 0.0203 | 0.0852 | 0.0315 | 0.0104 | 0.0746 | 0.0475 | 0.0441 | 0.0346 | 0.0954 | 0.0695 | 0.0336 | 0.0027 | 0.0299 | |
| 178 | N | 0.0881 | 0.0067 | 0.0969 | 0.0732 | 0.0171 | 0.2135 | 0.0283 | 0.014 | 0.06 | 0.03 | 0.0051 | 0.0666 | 0.0468 | 0.0328 | 0.0236 | 0.0757 | 0.046 | 0.02393 | 0.0195 | 0.0285 | |
| 179 | N | 0.0821 | 0.0091 | 0.0591 | 0.0594 | 0.0226 | 0.0625 | 0.0412 | 0.0308 | 0.1095 | 0.0747 | 0.015 | 0.0587 | 0.0356 | 0.051 | 0.0735 | 0.0732 | 0.0682 | 0.0507 | 0.0033 | 0.0233 | |
| 180 | R | 0.0586 | 0.0106 | 0.0203 | 0.0239 | 0.0217 | 0.0338 | 0.0348 | 0.0771 | 0.1127 | 0.1198 | 0.026 | 0.0278 | 0.0306 | 0.0331 | 0.1377 | 0.0524 | 0.0522 | 0.1143 | 0.0054 | 0.0094 | |
| 181 | A | 0.1794 | 0.0218 | 0.037 | 0.0408 | 0.017 | 0.0973 | 0.0174 | 0.0217 | 0.0423 | 0.0352 | 0.0074 | 0.0373 | 0.1284 | 0.0255 | 0.0219 | 0.1159 | 0.0803 | 0.0511 | 0.0024 | 0.0252 | |
| 182 | S | 0.0875 | 0.0153 | 0.0549 | 0.0482 | 0.0454 | 0.0689 | 0.0239 | 0.0179 | 0.0529 | 0.0385 | 0.0067 | 0.0443 | 0.0582 | 0.0259 | 0.0438 | 0.1031 | 0.0653 | 0.0337 | 0.1118 | 0.0563 | |
| 183 | F | 0.0235 | 0.0111 | 0.0065 | 0.0073 | 0.3931 | 0.0134 | 0.0198 | 0.0307 | 0.012 | 0.0798 | 0.0077 | 0.015 | 0.0105 | 0.0063 | 0.0109 | 0.0236 | 0.0175 | 0.0253 | 0.0388 | 0.2531 | |
| 184 | S | 0.1258 | 0.0236 | 0.0468 | 0.0406 | 0.0155 | 0.104 | 0.0179 | 0.0197 | 0.0587 | 0.0528 | 0.0091 | 0.0531 | 0.0598 | 0.0235 | 0.0281 | 0.1715 | 0.1001 | 0.0383 | 0.0047 | 0.0102 | |
| 185 | Q | 0.0942 | 0.012 | 0.0938 | 0.1014 | 0.0112 | 0.0828 | 0.0389 | 0.0172 | 0.0781 | 0.0291 | 0.0066 | 0.0901 | 0.041 | 0.0536 | 0.0264 | 0.1111 | 0.0722 | 0.0292 | 0.0024 | 0.0117 | |
| 186 | Y | 0.0638 | 0.0144 | 0.0289 | 0.0359 | 0.0882 | 0.0411 | 0.0333 | 0.0256 | 0.0817 | 0.044 | 0.0098 | 0.0305 | 0.0304 | 0.0286 | 0.0828 | 0.0555 | 0.0447 | 0.0471 | 0.048 | 0.1637 | |
| 187 | G | 0.09 | 0.1875 | 0.0387 | 0.0319 | 0.0105 | 0.3251 | 0.0116 | 0.0117 | 0.0287 | 0.0151 | 0.003 | 0.0364 | 0.026 | 0.0142 | 0.0101 | 0.0782 | 0.038 | 0.0288 | 0.0009 | 0.0142 | |
| 188 | A | 0.1211 | 0.0278 | 0.0425 | 0.0416 | 0.013 | 0.0912 | 0.0248 | 0.0195 | 0.068 | 0.0373 | 0.0094 | 0.0468 | 0.1222 | 0.0293 | 0.0483 | 0.1218 | 0.083 | 0.0423 | 0.0036 | 0.0128 | |
| 189 | G | 0.0967 | 0.0868 | 0.0538 | 0.0636 | 0.019 | 0.0961 | 0.0219 | 0.0307 | 0.0561 | 0.0428 | 0.0087 | 0.0436 | 0.0382 | 0.0377 | 0.0356 | 0.0947 | 0.0665 | 0.0692 | 0.0205 | 0.0202 | |
| 190 | L | 0.0872 | 0.0124 | 0.0207 | 0.0248 | 0.0286 | 0.0428 | 0.0145 | 0.0841 | 0.0372 | 0.1508 | 0.021 | 0.0241 | 0.0862 | 0.0216 | 0.0189 | 0.0589 | 0.0813 | 0.1654 | 0.0071 | 0.0197 | |
| 191 | D | 0.0718 | 0.0275 | 0.1459 | 0.1181 | 0.0232 | 0.0659 | 0.0271 | 0.0255 | 0.0525 | 0.1215 | 0.0159 | 0.0631 | 0.0261 | 0.0436 | 0.0161 | 0.0575 | 0.0442 | 0.0381 | 0.0069 | 0.0115 | |
| 192 | I | 0.1003 | 0.0172 | 0.0203 | 0.0256 | 0.0433 | 0.0464 | 0.0108 | 0.1316 | 0.0309 | 0.1616 | 0.0222 | 0.0205 | 0.0297 | 0.0169 | 0.0161 | 0.0468 | 0.0594 | 0.185 | 0.0011 | 0.0195 | |
| 193 | V | 0.105 | 0.0197 | 0.0248 | 0.026 | 0.1206 | 0.0595 | 0.0145 | 0.0529 | 0.0421 | 0.0833 | 0.0271 | 0.0298 | 0.0363 | 0.0172 | 0.0187 | 0.0843 | 0.0869 | 0.107 | 0.0036 | 0.0474 | |
| 194 | A | 0.2119 | 0.0126 | 0.0409 | 0.0444 | 0.0202 | 0.1126 | 0.0149 | 0.0254 | 0.0445 | 0.0341 | 0.0086 | 0.0384 | 0.0593 | 0.0236 | 0.0172 | 0.1067 | 0.1181 | 0.0594 | 0.0015 | 0.0114 | |
| 195 | P | 0.0917 | 0.0141 | 0.0198 | 0.0263 | 0.0672 | 0.0407 | 0.0238 | 0.0168 | 0.0329 | 0.036 | 0.0048 | 0.024 | 0.3087 | 0.0285 | 0.0245 | 0.0701 | 0.0433 | 0.032 | 0.003 | 0.1033 | |
| 196 | G | 0.1194 | 0.0102 | 0.0447 | 0.0388 | 0.011 | 0.3768 | 0.0113 | 0.0114 | 0.0382 | 0.0186 | 0.0043 | 0.0431 | 0.0379 | 0.0179 | 0.014 | 0.1098 | 0.0558 | 0.0312 | 0.0019 | 0.0057 | |
| 197 | V | 0.1115 | 0.0149 | 0.0475 | 0.0544 | 0.0148 | 0.1253 | 0.0207 | 0.043 | 0.0496 | 0.0585 | 0.0114 | 0.0436 | 0.0416 | 0.0387 | 0.0216 | 0.1013 | 0.0814 | 0.108 | 0.0022 | 0.0133 | |
| 198 | N | 0.0903 | 0.0093 | 0.0854 | 0.0764 | 0.0207 | 0.1212 | 0.033 | 0.017 | 0.0924 | 0.0405 | 0.0108 | 0.0698 | 0.0352 | 0.0493 | 0.0357 | 0.0841 | 0.0542 | 0.0304 | 0.0083 | 0.0379 | |
| 199 | V | 0.0647 | 0.0121 | 0.0159 | 0.0212 | 0.0431 | 0.0285 | 0.009 | 0.2056 | 0.0337 | 0.1344 | 0.0274 | 0.018 | 0.0197 | 0.0143 | 0.0187 | 0.0334 | 0.0543 | 0.227 | 0.0069 | 0.0186 | |
| 200 | Q | 0.0756 | 0.0096 | 0.0229 | 0.0283 | 0.0533 | 0.0419 | 0.021 | 0.0578 | 0.0494 | 0.2161 | 0.0225 | 0.026 | 0.0368 | 0.0294 | 0.0259 | 0.0479 | 0.058 | 0.1007 | 0.0199 | 0.0598 | |
| 201 | S | 0.1339 | 0.0224 | 0.0409 | 0.0375 | 0.0137 | 0.1097 | 0.0174 | 0.0216 | 0.0611 | 0.0271 | 0.0083 | 0.0515 | 0.0562 | 0.0239 | 0.0272 | 0.167 | 0.1287 | 0.0437 | 0.0042 | 0.01 | |
| 202 | T | 0.1468 | 0.0201 | 0.0356 | 0.036 | 0.0141 | 0.074 | 0.0149 | 0.0363 | 0.0585 | 0.0517 | 0.0132 | 0.0413 | 0.0474 | 0.0219 | 0.0205 | 0.1169 | 0.1776 | 0.0713 | 0.018 | 0.0102 | |
| 203 | Y | 0.0546 | 0.0087 | 0.0711 | 0.0525 | 0.0439 | 0.0464 | 0.0234 | 0.0374 | 0.0411 | 0.0787 | 0.0094 | 0.0381 | 0.0291 | 0.0255 | 0.0387 | 0.0475 | 0.0365 | 0.0625 | 0.1805 | 0.0744 | |
| 204 | P | 0.0779 | 0.0101 | 0.0314 | 0.0326 | 0.0395 | 0.0492 | 0.023 | 0.069 | 0.0457 | 0.1697 | 0.0192 | 0.0347 | 0.1066 | 0.0283 | 0.0246 | 0.06 | 0.0559 | 0.088 | 0.0077 | 0.0334 | |
| 205 | G | 0.0864 | 0.0141 | 0.0697 | 0.0597 | 0.0147 | 0.1655 | 0.0503 | 0.0194 | 0.0676 | 0.0409 | 0.0086 | 0.0631 | 0.0516 | 0.0456 | 0.0425 | 0.0804 | 0.0592 | 0.0372 | 0.0081 | 0.0179 | |
| 206 | S | 0.0956 | 0.011 | 0.0677 | 0.0588 | 0.0151 | 0.1439 | 0.0291 | 0.021 | 0.0906 | 0.0273 | 0.0075 | 0.0643 | 0.0524 | 0.0392 | 0.0478 | 0.0976 | 0.075 | 0.0365 | 0.0029 | 0.0209 | |
| 207 | T | 0.0928 | 0.169 | 0.0398 | 0.04 | 0.0184 | 0.1341 | 0.0218 | 0.0179 | 0.068 | 0.0263 | 0.0063 | 0.0372 | 0.0335 | 0.0265 | 0.0475 | 0.0819 | 0.0599 | 0.036 | 0.0087 | 0.0363 | |
| 208 | Y | 0.0744 | 0.0228 | 0.0216 | 0.022 | 0.1117 | 0.0367 | 0.0211 | 0.0294 | 0.0408 | 0.0581 | 0.0105 | 0.0318 | 0.0285 | 0.0178 | 0.0152 | 0.0694 | 0..1141 | 0.0451 | 0.011 | 0.2193 | |
| 209 | A | 0.1101 | 0.0141 | 0.0713 | 0.066 | 0.0133 | 0.1056 | 0.0337 | 0.0276 | 0.0771 | 0.0633 | 0.0131 | 0.0646 | 0.0388 | 0.0462 | 0.0397 | 0.0815 | 0.0752 | 0.0503 | 0.0018 0.0101 | | |
| 210 | S | 0.0994 | 0.0144 | 0.0399 | 0.0427 | 0.0361 | 0.0719 | 0.0196 | 0.0434 | 0.0812 | 0.0728 | 0.0203 | 0.0436 | 0.0386 | 0.0297 | 0.0296 | 0.1003 | 0.1095 | 0.065 | 0.0029 | 0.0448 | |
| 211 | L | 0.0469 | 0.0084 | 0.0249 | 0.0286 | 0.0762 | 0.033 | 0.0877 | 0.0814 | 0.0555 | 0.1689 | 0.0429 | 0.032 | 0.024 | 0.0398 | 0.03 | 0.0355 | 0.0361 | 0.0883 | 0.0081 | 0.0528 | |
| 212 | N | 0.1162 | 0.0188 | 0.0544 | 0.0455 | 0.0189 | 0.1087 | 0.025 | 0.0217 | 0.0696 | 0.0292 | 0.0095 | 0.0646 | 0.0496 | 0.0316 | 0.0277 | 0.1467 | 0.1096 | 0.0385 | 0.0037 | 0.0156 | |
| 213 | G | 0.1131 | 0.005 | 0.0446 | 0.0384 | 0.0099 | 0.47 | 0.0086 | 0.0093 | 0.0302 | 0.0167 | 0.0032 | 0.0382 | 0.0299 | 0.016 | 0.0086 | 0.0846 | 0.0405 | 0.0295 | 0.0008 | 0.0041 | |
| 214 | T | 0.1296 | 0.0136 | 0.035 | 0.0318 | 0.0131 | 0.0615 | 0.0158 | 0.0347 | 0.0695 | 0.0366 | 0.0116 | 0.0461 | 0.0426 | 0.0211 | 0.021 | 0.1209 | 0.2378 | 0.0608 | 0.0015 | 0.0102 | |
| 215 | S | 0.1278 | 0.0272 | 0.0429 | 0.0378 | 0.0143 | 0.1094 | 0.0182 | 0.0167 | 0.0622 | 0.0228 | 0.0073 | 0.0563 | 0.0613 | 0.0227 | 0.0309 | 0.194 | 0.1023 | 0.0338 | 0.0055 | 0.0103 | |
| 216 | M | 0.0936 | 0.0065 | 0.0203 | 0.0269 | 0.0429 | 0.0448 | 0.0114 | 0.0535 | 0.0736 | 0.1737 | 0.1448 | 0.0207 | 0.0301 | 0.0254 | 0.0261 | 0.0502 | 0.0513 | 0.0896 | 0.0013 | 0.0126 | |
| 217 | A | 0.2237 | 0.0155 | 0.0429 | 0.047 | 0.0121 | 0.1133 | 0.0152 | 0.022 | 0.0426 | 0.0309 | 0.0079 | 0.0398 | 0.0656 | 0.0246 | 0.0192 | 0.1216 | 0.0916 | 0.0562 | 0.0021 | 0.0094 | |
| 218 | T | 0.1645 | 0.0549 | 0.0367 | 0.0371 | 0.0126 | 0.0887 | 0.015 | 0.0273 | 0.0523 | 0.0359 | 0.0093 | 0.0408 | 0.0524 | 0.0216 | 0.0202 | 0.1223 | 0.1462 | 0.057 | 0.002 | 0.0106 | |
| 219 | P | 0.1142 | 0.0125 | 0.0242 | 0.032 | 0.0051 | 0.0547 | 0.0237 | 0.0105 | 0.0394 | 0.0291 | 0.0043 | 0.0263 | 0.4001 | 0.0353 | 0.0299 | 0.0877 | 0.0517 | 0.0313 | 0.0012 | 0.0039 | |
| 220 | H | 0.0431 | 0.0082 | 0.0378 | 0.0414 | 0.0456 | 0.0323 | 0.2111 | 0.0282 | 0.0497 | 0.1391 | 0.0172 | 0.0522 | 0.0313 | 0.0648 | 0.0404 | 0.036 | 0.028 | 0.0476 | 0.0017 | 0.041 | |
| 221 | V | 0.1329 | 0.0143 | 0.0246 | 0.03 | 0.0215 | 0.0638 | 0.0113 | 0.0975 | 0.0301 | 0.0957 | 0.0183 | 0.0227 | 0.0368 | 0.0176 | 0.0148 | 0.0573 | 0.0679 | 0.2344 | 0.0008 | 0.0113 | |
| 222 | A | 0.2051 | 0.0207 | 0.0398 | 0.0434 | 0.0137 | 0.102 | 0.0148 | 0.029 | 0.0435 | 0.0362 | 0.009 | 0.0381 | 0.0599 | 0.0233 | 0.0185 | 0.1132 | 0.1051 | 0.0732 | 0.0019 | 0.0138 | |
| 223 | G | 0.1221 | 0.0056 | 0.0444 | 0.0391 | 0.01 | 0.4435 | 0.009 | 0.0104 | 0.031 | 0.0179 | 0.0036 | 0.0381 | 0.0324 | 0.0166 | 0.0092 | 0.0861 | 0.0453 | 0.0319 | 0.0008 | 0.0045 | |
| 224 | A | 0.1088 | 0.0174 | 0.0228 | 0.0272 | 0.0274 | 0.0599 | 0.0117 | 0.0972 | 0.033 | 0.1775 | 0.0262 | 0.0231 | 0.0331 | 0.0184 | 0.016 | 0.016 | 0.0549 | 0.0604 | 0.1718 | 0.001 | 0.0157 |
| 225 | A | 0.1433 | 0.0229 | 0.0291 | 0.0348 | 0.0657 | 0.0792 | 0.0142 | 0.0608 | 0.0347 | 0.1137 | 0.0179 | 0.027 | 0.0422 | 0.0218 | 0.0188 | 0.0709 | 0.0658 | 0.1055 | 0.0021 | 0.0335 | |
| 226 | A | 0.2137 | 0.0119 | 0.0394 | 0.0444 | 0.0141 | 0.1199 | 0.0139 | 0.0295 | 0.0387 | 0.0654 | 0.0119 | 0.0344 | 0.059 | 0.0237 | 0.0163 | 0.0974 | 0.0903 | 0.0692 | 0.0013 | 0.0094 | |
| 227 | L | 0.0421 | 0.0132 | 0.0099 | 0.0146 | 0.0738 | 0.0202 | 0.0138 | 0.0552 | 0.0277 | 0.3992 | 0.0303 | 0.0144 | 0.0187 | 0.0174 | 0.0132 | 0.0239 | 0.0288 | 0.0826 | 0.0023 | 0.0982 | |
| 228 | V | 0.086 | 0.0099 | 0.0185 | 0.0243 | 0.0584 | 0.0403 | 0.0123 | 0.0906 | 0.0312 | 0.2369 | 0.0331 | 0.0193 | 0.0275 | 0.0182 | 0.0152 | 0.0418 | 0.0512 | 0.01368 | 0.0017 | 0.0493 | |
| 229 | K | 0.055 | 0.0055 | 0.0187 | 0.0253 | 0.0396 | 0.0324 | 0.0175 | 0.0653 | 0.1002 | 0.278 | 0.0386 | 0.0226 | 0.0251 | 0.0339 | 0.0476 | 0.0375 | 0.0395 | 0.083 | 0.0257 | 0.0131 | |
| 230 | Q | 0.113 | 0.0127 | 0.0791 | 0.1113 | 0.01 | 0.0968 | 0.0312 | 0.0185 | 0.0633 | 0.0316 | 0.0074 | 0.0499 | 0.0509 | 0.0797 | 0.0282 | 0.1057 | 0.0682 | 0.0346 | 0.0024 | 0.0083 | |
| 231 | K | 0.11 | 0.0093 | 0.0348 | 0.0437 | 0.0269 | 0.0765 | 0.0255 | 0.0364 | 0.0986 | 0.1226 | 0.017 | 0.0327 | 0.0388 | 0.0457 | 0.049 | 0.0631 | 0.0584 | 0.0679 | 0.0084 | 0.0379 | |
| 232 | N | 0.0742 | 0.0088 | 0.0667 | 0.0623 | 0.0575 | 0.0827 | 0.0707 | 0.0225 | 0.0726 | 0.0596 | 0.0082 | 0.0748 | 0.0375 | 0.0475 | 0.0394 | 0.067 | 0.0475 | 0.0368 | 0.0029 | 0.0619 | |
| 233 | P | 0.0934 | 0.01 | 0.0444 | 0.0506 | 0.0079 | 0.076 | 0.0284 | 0.0172 | 0.0881 | 0.0426 | 0.0076 | 0.0415 | 0.2241 | 0.0442 | 0.0596 | 0.0776 | 0.0492 | 0.0403 | 0.0023 | 0.0057 | |
| 234 | S | 0.0861 | 0.017 | 0.0722 | 0.0673 | 0.0328 | 0.0712 | 0.0354 | 0.0243 | 0.0881 | 0.0807 | 0.011 | 0.064 | 0.0352 | 0.0429 | 0.0306 | 0.0855 | 0.0741 | 0.0423 | 0.0025 | 0.0405 | |
| 235 | W | 0.0677 | 0.0189 | 0.0231 | 0.0278 | 0.0382 | 0.0391 | 0.0144 | 0.0433 | 0.0355 | 0.2765 | 0.0287 | 0.0215 | 0.0446 | 0.0214 | 0.0242 | 0.0457 | 0.0412 | 0.066 | 0.0895 | 0.0346 | |
| 236 | S | 0.1182 | 0.0164 | 0.0492 | 0.0439 | 0.0131 | 0.0859 | 0.0196 | 0.0259 | 0.0747 | 0.0381 | 0.0099 | 0.0525 | 0.0493 | 0.0271 | 0.0335 | 0.1335 | 0.159 | 0.0465 | 0.003 | 0.0097 | |
| 237 | N | 0.0975 | 0.0092 | 0.0366 | 0.0397 | 0.0141 | 0.0631 | 0.0225 | 0.0197 | 0.0428 | 0.0395 | 0.006 | 0.0392 | 0.1578 | 0.0271 | 0.0345 | 0.0739 | 0.0502 | 0.0456 | 0.1776 | 0.0107 | |
| 238 | V | 0.1128 | 0.0127 | 0.0544 | 0.0555 | 0.0143 | 0.072 | 0.0311 | 0.0286 | 0.0907 | 0.0379 | 0.0095 | 0.0441 | 0.0537 | 0.0434 | 0.1032 | 0.0888 | 0.0691 | 0.0592 | 0.0049 | 0.0164 | |
| 239 | Q | 0.0967 | 0.0064 | 0.1125 | 0.1197 | 0.0182 | 0.0775 | 0.0367 | 0.0225 | 0.0659 | 0.0487 | 0.0147 | 0.0554 | 0.0384 | 0.0904 | 0.0288 | 0.0673 | 0.0549 | 0.0369 | 0.0013 | 0.0097 | |
| 240 | I | 0.0783 | 0.0106 | 0.0184 | 0.0231 | 0.031 | 0.0377 | 0.0103 | 0.1249 | 0.0354 | 0.2085 | 0.0437 | 0.0183 | 0.0279 | 0.0176 | 0.0172 | 0.0385 | 0.0507 | 0.1988 | 0.0008 | 0.0118 | |
| 241 | R | 0.0635 | 0.0482 | 0.0388 | 0.0574 | 0.0257 | 0.0399 | 0.0412 | 0.0238 | 0.1353 | 0.0496 | 0.0112 | 0.0337 | 0.0376 | 0.0854 | 0.1437 | 0.0583 | 0.0468 | 0.0374 | 0.0057 | 0.0188 | |
| 242 | N | 0.0949 | 0.0106 | 0.0761 | 0.0737 | 0.0199 | 0.0711 | 0.0857 | 0.0185 | 0.0866 | 0.0308 | 0.0089 | 0.0608 | 0.0403 | 0.061 | 0.0517 | 0.0779 | 0.0597 | 0.0336 | 0.0027 | 0.0364 | |
| 243 | H | 0.0848 | 0.0095 | 0.0302 | 0.0396 | 0.0366 | 0.0444 | 0.0342 | 0.0698 | 0.0708 | 0.1634 | 0.0204 | 0.0281 | 0.0335 | 0.0384 | 0.069 | 0.0522 | 0.0517 | 0.0972 | 0.0032 | 0.0254 | |
| 244 | L | 0.0613 | 0.0081 | 0.0156 | 0.0211 | 0.0462 | 0.0296 | 0.0107 | 0.113 | 0.034 | 0.3015 | 0.0397 | 0.0178 | 0.023 | 0.0184 | 0.0168 | 0.0375 | 0.053 | 0.1409 | 0.0011 | 0.0151 | |
| 245 | K | 0.0744 | 0.0095 | 0.0411 | 0.0548 | 0.0238 | 0.0453 | 0.0208 | 0.0694 | 0.1158 | 0.1109 | 0.0334 | 0.0352 | 0.0297 | 0.0424 | 0.0445 | 0.0556 | 0.0592 | 0.1234 | 0.0018 | 0.0142 | |
| 246 | N | 0.0792 | 0.0103 | 0.0551 | 0.059 | 0.0262 | 0.0592 | 0.0437 | 0.0222 | 0.1074 | 0.0639 | 0.0153 | 0.0562 | 0.0365 | 0.054 | 0.0705 | 0.0795 | 0.0699 | 0.0377 | 0.0215 | 0.0359 | |
| 247 | T | 0.1069 | 0.0131 | 0.0358 | 0.0331 | 0.0242 | 0.0654 | 0.0226 | 0.0328 | 0.0799 | 0.0769 | 0.0144 | 0.0466 | 0.0398 | 0.0246 | 0.0449 | 0.1059 | 0.1618 | 0.0538 | 0.0029 | 0.0238 | |
| 248 | A | 0.2046 | 0.0209 | 0.0442 | 0.046 | 0.0124 | 0.1157 | 0.0158 | 0.0247 | 0.0452 | 0.0318 | 0.0081 | 0.0421 | 0.0611 | 0.0242 | 0.0194 | 0.1199 | 0.0984 | 0.0577 | 0.0021 | 0.0096 | |
| 249 | T | 0.086 | 0.0113 | 0.0545 | 0.0551 | 0.022 | 0.0536 | 0.0277 | 0.0423 | 0.1036 | 0.0582 | 0.0125 | 0.0466 | 0.0384 | 0.0404 | 0.066 | 0.0826 | 0.1078 | 0.0722 | 0.0032 | 0.0225 | |
| 250 | S | 0.0817 | 0.0106 | 0.061 | 0.0582 | 0.023 | 0.0606 | 0.0305 | 0.0215 | 0.1095 | 0.0452 | 0.0121 | 0.0469 | 0.1066 | 0.0415 | 0.0751 | 0.0763 | 0.0568 | 0.0396 | 0.0156 | 0.0332 | |
| 251 | L | 0.0768 | 0.0068 | 0.0472 | 0.0413 | 0.0524 | 0.0996 | 0.0183 | 0.0606 | 0.0504 | 0.2007 | 0.0271 | 0.0399 | 0.0267 | 0.024 | 0.0227 | 0.0528 | 0.0481 | 0.0851 | 0.0016 | 0.0214 | |
| 252 | G | 0.0942 | 0.0095 | 0.0702 | 0.062 | 0.0152 | 0.1536 | 0.0348 | 0.0271 | 0.0659 | 0.0378 | 0.0076 | 0.0587 | 0.0731 | 0.0389 | 0.0337 | 0.0792 | 0.0528 | 0.0601 | 0.0079 | 0.0212 | |
| 253 | S | 0.0903 | 0.0248 | 0.0502 | 0.0447 | 0.0565 | 0.0589 | 0.0274 | 0.0592 | 0.0581 | 0.0656 | 0.0122 | 0.0472 | 0.0515 | 0.026 | 0.0323 | 0.0785 | 0.0704 | 0.1078 | 0.0029 | 0.0401 | |
| 254 | T | 0.1014 | 0.0134 | 0.0661 | 0.0564 | 0.0128 | 0.106 | 0.0296 | 0.0293 | 0.0713 | 0.0506 | 0.0105 | 0.0571 | 0.0947 | 0.0334 | 0.0347 | 0.1044 | 0.0683 | 0.054 | 0.0025 | 0.009 | |
| 255 | N | 0.0684 | 0.0106 | 0.041 | 0.0389 | 0.0921 | 0.0548 | 0.0822 | 0.0269 | 0.0786 | 0.0715 | 0.01 | 0.0507 | 0.0431 | 0.0424 | 0.0591 | 0.0719 | 0.0558 | 0.0402 | 0.0222 | 0.0433 | |
| 256 | L | 0.0825 | 0.0101 | 0.0519 | 0.0507 | 0.0653 | 0.12 | 0.0339 | 0.0275 | 0.0652 | 0.0921 | 0.0141 | 0.0401 | 0.0325 | 0.0388 | 0.0463 | 0.0688 | 0.0495 | 0.0447 | 0.0037 | 0.0638 | |
| 257 | Y | 0.0611 | 0.0255 | 0.0252 | 0.0336 | 0.1682 | 0.0378 | 0.033 | 0.0306 | 0.0434 | 0.0695 | 0.011 | 0.0292 | 0.0276 | 0.0464 | 0.0219 | 0.06 | 0.0565 | 0.043 | 0.0115 | 0.1673 | |
| 258 | G | 0.1102 | 0.0067 | 0.0394 | 0.0368 | 0.0103 | 0.3629 | 0.0119 | 0.0179 | 0.0394 | 0.0333 | 0.0055 | 0.035 | 0.0798 | 0.0212 | 0.0166 | 0.0802 | 0.0433 | 0.0477 | 0.001 | 0.0047 | |
| 259 | S | 0.0742 | 0.0121 | 0.0471 | 0.0365 | 0.1086 | 0.0535 | 0.0521 | 0.02 | 0.0696 | 0.0398 | 0.0068 | 0.0641 | 0.0293 | 0.0277 | 0.0277 | 0.0712 | 0.0509 | 0.0307 | 0.0584 | 0.1209 | |
| 260 | G | 0.1008 | 0.0058 | 0.0403 | 0.0374 | 0.0168 | 0.3765 | 0.0112 | 0.0187 | 0.0489 | 0.0565 | 0.0072 | 0.0357 | 0.0288 | 0.0199 | 0.0189 | 0.0746 | 0.0401 | 0.0441 | 0.0013 | 0.0178 | |
| 261 | L | 0.0544 | 0.007 | 0.0222 | 0.0277 | 0.0434 | 0.0297 | 0.0294 | 0.0671 | 0.0644 | 0.3004 | 0.0328 | 0.0223 | 0.025 | 0.0274 | 0.0514 | 0.0356 | 0.0365 | 0.1003 | 0.0023 | 0.0225 | |
| 262 | V | 0.0845 | 0.0097 | 0.0237 | 0.0266 | 0.0304 | 0.0414 | 0.0141 | 0.1078 | 0.0351 | 0.2251 | 0.0405 | 0.0211 | 0.0269 | 0.0195 | 0.0168 | 0.0403 | 0.0499 | 0.1766 | 0.0008 | 0.0119 | |
| 263 | N | 0.0635 | 0.0089 | 0.1214 | 0.0858 | 0.059 | 0.0601 | 0.0417 | 0.0182 | 0.0814 | 0.0283 | 0.0058 | 0.0838 | 0.0254 | 0.0443 | 0.0353 | 0.0636 | 0.0466 | 0.0249 | 0.0033 | 0.0988 | |
| 264 | A | 0.1526 | 0.0117 | 0.0429 | 0.0412 | 0.0195 | 0.0831 | 0.0211 | 0.0456 | 0.0468 | 0.0941 | 0.016 | 0.0478 | 0.0667 | 0.0247 | 0.0188 | 0.0834 | 0.0722 | 0.095 | 0.0014 | 0.0191 | |
| 265 | E | 0.0931 | 0.009 | 0.0575 | 0.064 | 0.0334 | 0.1589 | 0.0236 | 0.0239 | 0.0742 | 0.07 | 0.0127 | 0.0462 | 0.0357 | 0.0343 | 0.0309 | 0.0751 | 0.0567 | 0.0448 | 0.0144 | 0.044 | |
| 266 | A | 0.1303 | 0.0103 | 0.0552 | 0.0564 | 0.0213 | 0.0944 | 0.0262 | 0.0218 | 0.1088 | 0.0428 | 0.0096 | 0.0515 | 0.0441 | 0.0364 | 0.067 | 0.0838 | 0.0682 | 0.0453 | 0.0033 | 0.0267 | |
| 267 | A | 0.1519 | 0.0096 | 0.0393 | 0.0425 | 0.0358 | 0.0958 | 0.016 | 0.0374 | 0.0501 | 0.1056 | 0.0423 | 0.0316 | 0.0518 | 0.031 | 0.0211 | 0.0776 | 0.0674 | 0.0684 | 0.0141 | 0.0136 | |
| 268 | T | 0.0949 | 0.0132 | 0.0325 | 0.0387 | 0.0307 | 0.0733 | 0.0151 | 0.0864 | 0.0413 | 0.108 | 0.0176 | 0.0274 | 0.03 | 0.0258 | 0.0179 | 0.0558 | 0.0622 | 0.1901 | 0.008 | 0.0341 | |
| 269 | R | 0.0808 | 0.0077 | 0.0851 | 0.0863 | 0.0216 | 0.067 | 0.0414 | 0.0231 | 0.1114 | 0.0453 | 0.0133 | 0.0628 | 0.0418 | 0.066 | 0.0518 | 0.0715 | 0.0658 | 0.0437 | 0.0022 | 0.016 | |

TABLE 2

| GG36 | A | C | D | E | F | G | H | I | K | L | M | N | P | Q | R | S | T | V | W | Y |
|---|---|---|---|---|---|---|---|---|---|---|---|---|---|---|---|---|---|---|---|---|
| A | 0.98 | 0.11 | 0.60 | 0.49 | 0.22 | 0.75 | 0.35 | 0.19 | 0.85 | 0.39 | 0.10 | 0.73 | 0.60 | 0.34 | 0.41 | 1.00 | 0.76 | 0.33 | 0.24 | 0.13 |
| Q | 0.56 | 0.04 | 1.00 | 0.80 | 0.06 | 0.72 | 0.31 | 0.11 | 0.51 | 0.25 | 0.06 | 0.49 | 0.36 | 0.54 | 0.29 | 0.46 | 0.30 | 0.21 | 0.01 | 0.05 |
| S | 0.81 | 0.08 | 0.26 | 0.26 | 0.11 | 0.44 | 0.13 | 0.16 | 0.28 | 0.33 | 0.05 | 0.23 | 1.00 | 0.20 | 0.14 | 0.57 | 0.54 | 0.33 | 0.01 | 0.08 |
| V | 0.63 | 0.09 | 0.28 | 0.33 | 0.49 | 0.42 | 0.25 | 0.37 | 0.74 | 1.00 | 0.17 | 0.29 | 0.33 | 0.29 | 0.40 | 0.51 | 0.51 | 0.72 | 0.10 | 0.40 |
| P | 0.56 | 0.05 | 0.29 | 0.43 | 0.06 | 0.35 | 0.25 | 0.14 | 0.46 | 0.29 | 0.12 | 0.23 | 1.00 | 0.63 | 0.25 | 0.43 | 0.39 | 0.27 | 0.01 | 0.06 |
| W | 0.06 | 0.01 | 0.03 | 0.03 | 0.08 | 0.04 | 0.03 | 0.02 | 0.06 | 0.07 | 0.01 | 0.04 | 0.05 | 0.03 | 0.10 | 0.08 | 0.05 | 0.03 | 1.00 | 0.12 |
| G | 0.37 | 0.05 | 0.22 | 0.19 | 0.20 | 1.00 | 0.20 | 0.07 | 0.19 | 0.17 | 0.02 | 0.19 | 0.16 | 0.12 | 0.11 | 0.29 | 0.18 | 0.15 | 0.01 | 0.48 |
| I | 0.17 | 0.03 | 0.05 | 0.06 | 0.23 | 0.08 | 0.04 | 0.26 | 0.09 | 1.00 | 0.12 | 0.05 | 0.12 | 0.06 | 0.05 | 0.10 | 0.13 | 0.34 | 0.01 | 0.18 |
| S | 0.80 | 0.07 | 0.56 | 0.58 | 0.30 | 1.00 | 0.45 | 0.21 | 0.84 | 0.63 | 0.09 | 0.48 | 0.54 | 0.47 | 0.42 | 0.65 | 0.47 | 0.36 | 0.02 | 0.20 |
| R | 0.75 | 0.19 | 0.45 | 0.44 | 0.19 | 0.75 | 0.36 | 0.24 | 0.90 | 0.49 | 0.16 | 0.43 | 0.39 | 0.43 | 1.00 | 0.64 | 0.48 | 0.39 | 0.04 | 0.20 |
| V | 0.49 | 0.07 | 0.16 | 0.18 | 0.21 | 0.46 | 0.08 | 0.80 | 0.25 | 0.76 | 0.12 | 0.16 | 0.19 | 0.12 | 0.16 | 0.33 | 0.44 | 1.00 | 0.01 | 0.10 |
| Q | 0.92 | 0.12 | 0.71 | 0.70 | 0.24 | 1.00 | 0.45 | 0.21 | 0.94 | 0.39 | 0.09 | 0.63 | 0.44 | 0.64 | 0.057 | 0.95 | 0.66 | 0.35 | 0.04 | 0.45 |
| A | 1.00 | 0.14 | 0.31 | 0.32 | 0.33 | 0.66 | 0.14 | 0.43 | 0.36 | 0.66 | 0.13 | 0.31 | 0.31 | 0.18 | 0.16 | 0.61 | 0.69 | 0.99 | 0.06 | 0.16 |
| P | 0.86 | 0.09 | 0.82 | 0.73 | 0.20 | 0.72 | 0.29 | 0.34 | 1.00 | 0.74 | 0.16 | 0.54 | 0.80 | 0.42 | 0.45 | 0.75 | 0.69 | 0.54 | 0.20 | 0.14 |
| A | 0.73 | 0.16 | 0.42 | 0.54 | 0.48 | 0.63 | 0.27 | 0.22 | 0.65 | 0.32 | 0.08 | 0.31 | 0.52 | 0.43 | 0.50 | 0.56 | 0.43 | 0.39 | 0.20 | 1.00 |
| A | 1.00 | 0.09 | 0.30 | 0.34 | 0.18 | 0.52 | 0.16 | 0.45 | 0.40 | 0.98 | 0.14 | 0.27 | 0.34 | 0.23 | 0.23 | 0.54 | 0.51 | 0.89 | 0.01 | 0.18 |
| H | 0.09 | 0.05 | 0.06 | 0.06 | 0.24 | 0.08 | 0.16 | 0.04 | 0.08 | 0.14 | 0.01 | 0.07 | 0.05 | 0.08 | 0.12 | 0.10 | 0.06 | 0.06 | 1.00 | 0.51 |
| N | 0.88 | 0.13 | 1.00 | 0.89 | 0.09 | 0.75 | 0.27 | 0.17 | 0.94 | 0.31 | 0.07 | 0.55 | 0.48 | 0.49 | 0.52 | 0.68 | 0.55 | 0.34 | 0.02 | 0.11 |
| R | 0.74 | 0.08 | 0.50 | 0.55 | 0.23 | 0.49 | 0.29 | 0.29 | 1.00 | 0.75 | 0.28 | 0.36 | 0.35 | 0.53 | 0.70 | 0.59 | 0.54 | 0.49 | 0.03 | 0.17 |
| G | 0.46 | 0.05 | 0.24 | 0.23 | 0.06 | 1.00 | 0.08 | 0.08 | 0.27 | 0.11 | 0.03 | 0.22 | 0.23 | 0.11 | 0.13 | 0.43 | 0.30 | 0.16 | 0.01 | 0.07 |
| L | 0.30 | 0.08 | 0.10 | 0.11 | 0.60 | 0.20 | 0.11 | 0.29 | 0.20 | 0.48 | 0.06 | 0.13 | 0.13 | 0.12 | 0.13 | 0.22 | 0.20 | 0.41 | 0.03 | 1.00 |
| T | 0.97 | 0.09 | 0.41 | 0.38 | 0.25 | 0.77 | 0.16 | 0.21 | 0.66 | 0.36 | 0.08 | 0.35 | 0.36 | 0.24 | 0.34 | 0.78 | 1.00 | 0.41 | 0.02 | 0.17 |
| G | 0.26 | 0.01 | 0.11 | 0.09 | 0.03 | 1.00 | 0.02 | 0.02 | 0.08 | 0.04 | 0.01 | 0.09 | 0.08 | 0.04 | 0.04 | 0.21 | 0.10 | 0.07 | 0.00 | 0.02 |
| S | 0.54 | 0.06 | 0.34 | 0.40 | 0.09 | 0.41 | 0.21 | 0.12 | 1.00 | 0.17 | 0.06 | 0.32 | 0.26 | 0.33 | 0.57 | 0.51 | 0.41 | 0.20 | 0.06 | 0.12 |
| G | 0.29 | 0.02 | 0.18 | 0.14 | 0.03 | 1.00 | 0.06 | 0.03 | 0.16 | 0.05 | 0.01 | 0.17 | 0.09 | 0.07 | 0.06 | 0.24 | 0.14 | 0.08 | 0.00 | 0.02 |
| V | 0.41 | 0.06 | 0.11 | 0.13 | 0.09 | 0.21 | 0.06 | 0.52 | 0.17 | 0.42 | 0.09 | 0.11 | 0.13 | 0.12 | 0.08 | 0.25 | 0.39 | 1.00 | 0.00 | 0.05 |
| K | 0.54 | 0.38 | 0.20 | 0.20 | 0.20 | 0.36 | 0.17 | 0.46 | 0.63 | 0.52 | 0.11 | 0.24 | 0.21 | 0.17 | 0.53 | 0.46 | 0.57 | 1.00 | 0.03 | 0.26 |
| V | 0.43 | 0.06 | 0.09 | 0.11 | 0.10 | 0.21 | 0.04 | 0.53 | 0.13 | 0.49 | 0.09 | 0.09 | 0.13 | 0.07 | 0.07 | 0.21 | 0.27 | 1.00 | 0.00 | 0.05 |
| A | 1.00 | 0.17 | 0.22 | 0.23 | 0.30 | 0.82 | 0.11 | 0.15 | 0.23 | 0.23 | 0.05 | 0.22 | 0.29 | 0.12 | 0.10 | 0.59 | 0.52 | 0.34 | 0.02 | 0.72 |
| V | 0.28 | 0.05 | 0.06 | 0.08 | 0.14 | 0.14 | 0.04 | 0.55 | 0.11 | 0.48 | 0.09 | 0.06 | 0.08 | 0.05 | 0.06 | 0.13 | 0.20 | 1.00 | 0.00 | 0.05 |
| L | 0.21 | 0.03 | 0.05 | 0.07 | 0.19 | 0.10 | 0.04 | 0.46 | 0.11 | 1.00 | 0.14 | 0.06 | 0.08 | 0.06 | 0.06 | 0.11 | 0.16 | 0.62 | 0.00 | 0.06 |
| D | 0.31 | 0.01 | 1.00 | 0.67 | 0.02 | 0.34 | 0.13 | 0.05 | 0.26 | 0.06 | 0.01 | 0.38 | 0.10 | 0.21 | 0.06 | 0.26 | 0.17 | 0.09 | 0.00 | 0.03 |
| T | 0.72 | 0.08 | 0.56 | 0.44 | 0.07 | 0.46 | 0.13 | 0.17 | 0.42 | 0.19 | 0.06 | 0.37 | 0.26 | 0.22 | 0.14 | 0.72 | 1.00 | 0.29 | 0.01 | 0.06 |
| G | 0.25 | 0.01 | 0.10 | 0.08 | 0.02 | 1.00 | 0.02 | 0.02 | 0.07 | 0.04 | 0.01 | 0.09 | 0.08 | 0.04 | 0.02 | 0.19 | 0.09 | 0.07 | 0.00 | 0.01 |
| I | 0.29 | 0.21 | 0.07 | 0.09 | 0.21 | 0.14 | 0.05 | 0.75 | 0.13 | 0.80 | 0.11 | 0.08 | 0.09 | 0.07 | 0.07 | 0.15 | 0.23 | 1.00 | 0.01 | 0.13 |
| S | 0.61 | 0.05 | 1.00 | 0.95 | 0.14 | 0.51 | 0.25 | 0.15 | 0.53 | 0.46 | 0.08 | 0.52 | 0.24 | 0.45 | 0.27 | 0.51 | 0.37 | 0.25 | 0.20 | 0.14 |
| T | 0.93 | 0.11 | 0.87 | 0.73 | 0.11 | 0.78 | 0.34 | 0.20 | 0.82 | 0.42 | 0.09 | 0.71 | 0.46 | 0.36 | 0.42 | 1.00 | 0.87 | 0.34 | 0.03 | 0.10 |
| H | 0.16 | 0.03 | 0.14 | 0.13 | 0.06 | 0.11 | 1.00 | 0.05 | 0.16 | 0.15 | 0.02 | 0.22 | 0.13 | 0.27 | 0.17 | 0.15 | 0.10 | 0.10 | 0.00 | 0.08 |
| P | 0.44 | 0.09 | 0.19 | 0.25 | 0.04 | 0.23 | 0.11 | 0.09 | 0.24 | 0.15 | 0.03 | 0.15 | 1.00 | 0.19 | 0.17 | 0.33 | 0.25 | 0.20 | 0.01 | 0.04 |
| D | 0.44 | 0.03 | 1.00 | 0.86 | 0.13 | 0.48 | 0.17 | 0.08 | 0.32 | 0.11 | 0.02 | 0.41 | 0.16 | 0.31 | 0.10 | 0.36 | 0.23 | 0.13 | 0.01 | 0.08 |
| L | 0.10 | 0.01 | 0.02 | 0.04 | 0.56 | 0.05 | 0.05 | 0.19 | 0.06 | 1.00 | 0.09 | 0.04 | 0.05 | 0.05 | 0.04 | 0.06 | 0.07 | 0.21 | 0.03 | 0.16 |
| N | 0.37 | 0.16 | 0.51 | 0.39 | 0.12 | 0.35 | 0.29 | 0.16 | 0.96 | 0.19 | 0.06 | 0.38 | 0.23 | 0.30 | 1.00 | 0.41 | 0.28 | 0.25 | 0.04 | 0.06 |
| I | 0.98 | 0.10 | 0.28 | 0.35 | 0.21 | 0.52 | 0.14 | 0.59 | 0.34 | 0.64 | 0.12 | 0.25 | 0.72 | 0.23 | 0.18 | 0.55 | 0.49 | 1.00 | 0.01 | 0.12 |
| R | 0.83 | 0.10 | 0.48 | 0.57 | 0.30 | 0.77 | 0.19 | 0.63 | 0.87 | 0.81 | 0.14 | 0.37 | 0.37 | 0.33 | 0.46 | 0.63 | 0.56 | 1.00 | 0.03 | 0.27 |
| G | 0.62 | 0.04 | 0.30 | 0.29 | 0.15 | 1.00 | 0.12 | 0.10 | 0.32 | 0.30 | 0.06 | 0.22 | 0.20 | 0.21 | 0.15 | 0.40 | 0.31 | 0.22 | 0.20 | 0.16 |
| G | 0.53 | 0.14 | 0.19 | 0.18 | 0.09 | 1.00 | 0.07 | 0.14 | 0.22 | 0.22 | 0.04 | 0.18 | 0.20 | 0.09 | 0.12 | 0.48 | 0.28 | 0.23 | 0.12 | 0.12 |
| A | 0.34 | 0.11 | 0.14 | 0.14 | 0.58 | 0.19 | 0.19 | 0.19 | 0.47 | 0.24 | 0.05 | 0.17 | 0.15 | 0.13 | 0.36 | 0.27 | 0.25 | 0.26 | 0.19 | 1.00 |
| S | 0.64 | 0.11 | 1.00 | 0.68 | 0.23 | 0.59 | 0.25 | 0.14 | 0.51 | 0.22 | 0.05 | 0.61 | 0.26 | 0.27 | 0.17 | 0.67 | 0.54 | 0.25 | 0.06 | 0.19 |
| F | 0.27 | 0.09 | 0.09 | 0.09 | 1.00 | 0.16 | 0.09 | 0.22 | 0.12 | 0.44 | 0.06 | 0.11 | 0.10 | 0.07 | 0.08 | 0.18 | 0.18 | 0.33 | 0.02 | 0.46 |
| V | 0.91 | 0.11 | 0.44 | 0.38 | 0.24 | 0.65 | 0.28 | 0.47 | 0.51 | 0.56 | 0.11 | 0.53 | 0.43 | 0.26 | 0.23 | 0.76 | 0.71 | 1.00 | 0.24 | 0.20 |
| P | 0.68 | 0.06 | 0.72 | 0.56 | 0.11 | 1.00 | 0.17 | 0.14 | 0.42 | 0.22 | 0.04 | 0.40 | 0.34 | 0.23 | 0.20 | 0.59 | 0.44 | 0.27 | 0.01 | 0.11 |
| G | 0.52 | 0.26 | 0.46 | 0.37 | 0.09 | 1.00 | 0.27 | 0.11 | 0.47 | 0.16 | 0.05 | 0.45 | 0.25 | 0.22 | 0.21 | 0.51 | 0.34 | 0.21 | 0.01 | 0.14 |
| E | 0.70 | 0.05 | 1.00 | 0.88 | 0.09 | 1.00 | 0.24 | 0.17 | 0.50 | 0.22 | 0.05 | 0.51 | 0.25 | 0.43 | 0.19 | 0.58 | 0.51 | 0.26 | 0.01 | 0.12 |
| P | 0.81 | 0.14 | 0.65 | 0.61 | 0.26 | 1.00 | 0.40 | 0.20 | 0.52 | 0.40 | 0.08 | 0.50 | 0.83 | 0.36 | 0.28 | 0.71 | 0.49 | 0.34 | 0.07 | 0.23 |
| S | 0.77 | 0.09 | 1.00 | 0.72 | 0.40 | 0.82 | 0.26 | 0.27 | 0.48 | 0.44 | 0.07 | 0.57 | 0.27 | 0.31 | 0.19 | 0.69 | 0.55 | 0.45 | 0.15 | 0.45 |
| T | 0.53 | 0.06 | 0.23 | 0.22 | 0.17 | 0.32 | 0.11 | 0.15 | 0.26 | 0.27 | 0.04 | 0.17 | 1.00 | 0.16 | 0.12 | 0.37 | 0.30 | 0.26 | 0.01 | 0.24 |
| Q | 1.00 | 0.12 | 0.60 | 0.59 | 0.22 | 0.93 | 0.34 | 0.36 | 0.79 | 0.81 | 0.19 | 0.49 | 0.44 | 0.51 | 0.59 | 0.92 | 0.89 | 0.70 | 0.16 | 0.15 |
| D | 0.49 | 0.08 | 1.00 | 0.68 | 0.09 | 0.49 | 0.20 | 0.10 | 0.41 | 0.15 | 0.05 | 0.50 | 0.19 | 0.25 | 0.15 | 0.46 | 0.32 | 0.18 | 0.15 | 0.08 |
| G | 0.57 | 0.24 | 0.52 | 0.50 | 0.29 | 1.00 | 0.14 | 0.13 | 0.35 | 0.33 | 0.05 | 0.32 | 0.21 | 0.23 | 0.20 | 0.52 | 0.34 | 0.24 | 0.02 | 0.25 |
| N | 0.78 | 0.09 | 0.91 | 0.70 | 0.19 | 0.83 | 0.53 | 0.26 | 0.82 | 0.57 | 0.10 | 1.00 | 0.36 | 0.46 | 0.26 | 0.87 | 0.61 | 0.43 | 0.02 | 0.18 |
| G | 0.34 | 0.07 | 0.15 | 0.14 | 0.06 | 1.00 | 0.08 | 0.05 | 0.23 | 0.09 | 0.02 | 0.15 | 0.14 | 0.10 | 0.23 | 0.29 | 0.16 | 0.11 | 0.12 | 0.08 |
| H | 0.10 | 0.03 | 0.12 | 0.12 | 0.05 | 0.08 | 1.00 | 0.03 | 0.14 | 0.10 | 0.01 | 0.20 | 0.11 | 0.27 | 0.16 | 0.11 | 0.07 | 0.07 | 0.00 | 0.08 |
| G | 0.24 | 0.01 | 0.09 | 0.08 | 0.02 | 1.00 | 0.02 | 0.02 | 0.06 | 0.04 | 0.01 | 0.08 | 0.06 | 0.03 | 0.02 | 0.18 | 0.09 | 0.06 | 0.00 | 0.01 |
| T | 0.57 | 0.06 | 0.15 | 0.14 | 0.06 | 0.28 | 0.07 | 0.15 | 0.30 | 0.17 | 0.06 | 0.19 | 0.19 | 0.10 | 0.09 | 0.53 | 1.00 | 0.27 | 0.01 | 0.04 |
| H | 0.17 | 0.04 | 0.15 | 0.16 | 0.09 | 0.14 | 1.00 | 0.06 | 0.34 | 0.15 | 0.03 | 0.23 | 0.16 | 0.33 | 0.51 | 0.19 | 0.12 | 0.11 | 0.02 | 0.10 |
| V | 0.29 | 1.00 | 0.06 | 0.07 | 0.05 | 0.15 | 0.04 | 0.33 | 0.09 | 0.30 | 0.07 | 0.06 | 0.09 | 0.05 | 0.05 | 0.20 | 0.22 | 0.90 | 0.00 | 0.06 |
| A | 1.00 | 0.16 | 0.18 | 0.20 | 0.05 | 0.49 | 0.06 | 0.11 | 0.17 | 0.14 | 0.03 | 0.16 | 0.28 | 0.10 | 0.07 | 0.47 | 0.39 | 0.26 | 0.01 | 0.04 |
| G | 0.27 | 0.02 | 0.10 | 0.09 | 0.02 | 1.00 | 0.02 | 0.02 | 0.080 | 0.04 | 0.01 | 0.09 | 0.08 | 0.04 | 0.02 | 0.21 | 0.11 | 0.07 | 0.00 | 0.01 |
| T | 0.74 | 0.08 | 0.41 | 0.58 | 0.12 | 0.41 | 0.13 | 0.50 | 0.41 | 0.44 | 0.10 | 0.29 | 0.28 | 0.28 | 0.15 | 0.62 | 1.00 | 0.66 | 0.01 | 0.07 |
| I | 0.48 | 0.06 | 0.10 | 0.13 | 0.14 | 0.22 | 0.05 | 0.74 | 0.16 | 0.59 | 0.12 | 0.10 | 0.15 | 0.08 | 0.08 | 0.23 | 0.29 | 1.00 | 0.00 | 0.06 |
| A | 1.00 | 0.09 | 0.21 | 0.22 | 0.06 | 0.94 | 0.07 | 0.12 | 0.19 | 0.20 | 0.04 | 0.18 | 0.29 | 0.11 | 0.08 | 0.52 | 0.40 | 0.31 | 0.01 | 0.04 |
| A | 0.86 | 0.05 | 0.19 | 0.20 | 0.05 | 1.00 | 0.06 | 0.09 | 0.18 | 0.15 | 0.06 | 0.17 | 0.24 | 0.11 | 0.07 | 0.47 | 0.34 | 0.23 | 0.01 | 0.04 |
| L | 0.78 | 0.14 | 0.38 | 0.40 | 0.20 | 0.57 | 0.25 | 0.40 | 1.00 | 0.93 | 0.15 | 0.42 | 0.31 | 0.34 | 0.50 | 0.63 | 0.64 | 0.77 | 0.02 | 0.13 |
| N | 0.68 | 0.06 | 0.82 | 0.62 | 0.13 | 1.00 | 0.37 | 0.14 | 0.63 | 0.24 | 0.08 | 0.78 | 0.39 | 0.30 | 0.18 | 0.66 | 0.45 | 0.25 | 0.01 | 0.10 |
| N | 0.66 | 0.07 | 0.86 | 0.60 | 0.16 | 0.67 | 0.40 | 0.18 | 0.73 | 0.38 | 0.06 | 1.00 | 0.26 | 0.30 | 0.20 | 0.76 | 0.57 | 0.30 | 0.08 | 0.26 |
| S | 0.55 | 0.05 | 0.33 | 0.28 | 0.08 | 1.00 | 0.15 | 0.08 | 0.29 | 0.12 | 0.03 | 0.32 | 0.24 | 0.14 | 0.14 | 0.53 | 0.37 | 0.16 | 0.01 | 0.05 |
| I | 0.70 | 0.11 | 0.34 | 0.36 | 0.66 | 0.57 | 0.22 | 0.69 | 0.58 | 0.75 | 0.13 | 0.33 | 0.33 | 0.24 | 0.39 | 0.54 | 0.62 | 1.00 | 0.03 | 0.55 |
| G | 0.33 | 0.87 | 0.14 | 0.13 | 0.04 | 1.00 | 0.05 | 0.05 | 0.16 | 0.08 | 0.02 | 0.12 | 0.11 | 0.07 | 0.07 | 0.31 | 0.16 | 0.12 | 0.00 | 0.04 |
| V | 0.66 | 0.08 | 0.18 | 0.19 | 0.21 | 0.90 | 0.11 | 0.49 | 0.28 | 0.54 | 0.17 | 0.19 | 0.21 | 0.14 | 0.16 | 0.45 | 0.49 | 1.00 | 0.01 | 0.23 |
| L | 0.67 | 0.12 | 0.25 | 0.28 | 0.48 | 0.42 | 0.23 | 0.48 | 0.38 | 0.87 | 0.12 | 0.25 | 0.23 | 0.21 | 0.27 | 0.44 | 0.44 | 1.00 | 0.03 | 0.97 |
| G | 0.25 | 0.01 | 0.10 | 0.08 | 0.02 | 1.00 | 0.02 | 0.02 | 0.07 | 0.04 | 0.01 | 0.08 | 0.07 | 0.03 | 0.02 | 0.18 | 0.09 | 0.06 | 0.00 | 0.01 |
| V | 0.31 | 0.05 | 0.06 | 0.08 | 0.11 | 0.18 | 0.04 | 0.46 | 0.11 | 0.51 | 0.11 | 0.07 | 0.09 | 0.06 | 0.06 | 0.15 | 0.21 | 1.00 | 0.00 | 0.05 |
| A | 1.00 | 0.06 | 0.20 | 0.22 | 0.06 | 0.50 | 0.07 | 0.12 | 0.19 | 0.25 | 0.05 | 0.17 | 0.28 | 0.11 | 0.09 | 0.46 | 0.38 | 0.29 | 0.01 | 0.04 |
| P | 0.39 | 0.07 | 0.11 | 0.13 | 0.45 | 0.19 | 0.14 | 0.09 | 0.46 | 0.19 | 0.04 | 0.15 | 1.00 | 0.15 | 0.33 | 0.33 | 0.23 | 0.15 | 0.03 | 0.63 |
| S | 0.54 | 0.05 | 0.61 | 0.49 | 0.10 | 1.00 | 0.26 | 0.11 | 0.90 | 0.19 | 0.05 | 0.55 | 0.22 | 0.27 | 0.39 | 0.56 | 0.36 | 0.18 | 0.02 | 0.07 |
| A | 1.00 | 0.13 | 0.20 | 0.23 | 0.07 | 0.51 | 0.09 | 0.21 | 0.21 | 0.25 | 0.05 | 0.18 | 0.29 | 0.13 | 0.09 | 0.51 | 0.49 | 0.51 | 0.01 | 0.05 |
| E | 0.41 | 0.05 | 0.42 | 0.42 | 0.05 | 0.38 | 0.26 | 0.10 | 1.00 | 0.16 | 0.06 | 0.37 | 0.22 | 0.39 | 0.66 | 0.48 | 0.35 | 0.16 | 0.06 | 0.05 |
| L | 0.18 | 0.03 | 0.04 | 0.06 | 0.13 | 0.08 | 0.03 | 0.45 | 0.09 | 1.00 | 0.10 | 0.05 | 0.07 | 0.05 | 0.05 | 0.09 | 0.14 | 0.57 | 0.02 | 0.04 |
| Y | 0.61 | 0.08 | 0.17 | 0.19 | 0.50 | 0.75 | 0.18 | 0.42 | 0.23 | 1.00 | 0.16 | 0.20 | 0.19 | 0.15 | 0.11 | 0.36 | 0.32 | 0.69 | 0.10 | 0.61 |
| A | 0.65 | 0.05 | 0.19 | 0.19 | 0.11 | 1.00 | 0.06 | 0.16 | 0.17 | 0.21 | 0.04 | 0.17 | 0.35 | 0.10 | 0.08 | 0.43 | 0.29 | 0.32 | 0.01 | 0.06 |
| V | 0.31 | 0.06 | 0.07 | 0.09 | 0.25 | 0.18 | 0.05 | 0.48 | 0.12 | 0.50 | 0.11 | 0.08 | 0.09 | 0.06 | 0.06 | 0.15 | 0.21 | 1.00 | 0.01 | 0.34 |
| K | 0.16 | 0.03 | 0.13 | 0.13 | 0.03 | 0.15 | 0.14 | 0.06 | 1.00 | 0.09 | 0.05 | 0.16 | 0.12 | 0.17 | 0.87 | 0.22 | 0.16 | 0.09 | 0.03 | 0.02 |
| V | 0.38 | 0.05 | 0.08 | 0.10 | 0.11 | 0.19 | 0.05 | 0.42 | 0.19 | 0.57 | 0.24 | 0.09 | 0.12 | 0.08 | 0.08 | 0.19 | 0.24 | 1.00 | 0.00 | 0.05 |
| L | 0.12 | 0.07 | 0.04 | 0.05 | 0.28 | 0.12 | 0.03 | 0.12 | 0.06 | 1.00 | 0.08 | 0.04 | 0.05 | 0.05 | 0.03 | 0.08 | 0.08 | 0.18 | 0.02 | 0.08 |
| G | 0.62 | 0.06 | 0.86 | 0.62 | 0.10 | 1.00 | 0.19 | 0.12 | 0.44 | 0.19 | 0.04 | 0.48 | 0.25 | 0.28 | 0.25 | 0.60 | 0.37 | 0.23 | 0.14 | 0.14 |
| A | 0.92 | 0.40 | 0.73 | 0.68 | 0.16 | 1.00 | 0.31 | 0.17 | 0.82 | 0.41 | 0.08 | 0.54 | 0.52 | 0.47 | 0.47 | 0.82 | 0.54 | 0.34 | 0.02 | 0.10 |
| S | 0.57 | 0.07 | 0.49 | 0.38 | 0.22 | 1.00 | 0.19 | 0.11 | 0.42 | 0.17 | 0.04 | 0.44 | 0.23 | 0.24 | 0.17 | 0.63 | 0.38 | 0.19 | 0.02 | 0.17 |
| G | 0.33 | 0.11 | 0.17 | 0.17 | 0.05 | 1.00 | 0.05 | 0.05 | 0.14 | 0.11 | 0.02 | 0.13 | 0.14 | 0.08 | 0.05 | 0.26 | 0.15 | 0.11 | 0.00 | 0.04 |
| S | 0.78 | 0.11 | 0.44 | 0.44 | 0.32 | 1.00 | 0.17 | 0.20 | 0.49 | 0.38 | 0.10 | 0.35 | 0.39 | 0.28 | 0.22 | 0.85 | 0.50 | 0.35 | 0.12 | 0.18 |
| G | 0.40 | 0.13 | 0.15 | 0.15 | 0.08 | 1.00 | 0.05 | 0.17 | 0.15 | 0.26 | 0.06 | 0.13 | 0.12 | 0.08 | 0.08 | 0.31 | 0.22 | 0.35 | 0.01 | 0.04 |
| S | 0.86 | 0.13 | 0.40 | 0.39 | 0.34 | 0.59 | 0.19 | 0.20 | 0.51 | 0.33 | 0.07 | 0.38 | 0.40 | 0.28 | 0.21 | 0.88 | 1.00 | 0.36 | 0.03 | 0.59 |
| V | 0.98 | 0.11 | 0.92 | 0.71 | 0.36 | 0.86 | 0.26 | 0.36 | 0.60 | 1.00 | 0.28 | 0.54 | 0.40 | 0.34 | 0.26 | 0.80 | 0.88 | 0.64 | 0.09 | 0.64 |
| S | 1.00 | 0.13 | 0.45 | 0.48 | 0.12 | 0.69 | 0.15 | 0.25 | 0.50 | 0.43 | 0.09 | 0.36 | 0.38 | 0.27 | 0.24 | 0.94 | 0.65 | 0.44 | 0.07 | 0.11 |
| S | 0.82 | 0.09 | 0.83 | 0.63 | 0.15 | 1.00 | 0.18 | 0.49 | 0.42 | 0.57 | 0.11 | 0.46 | 0.27 | 0.28 | 0.19 | 0.64 | 0.52 | 0.91 | 0.61 | 0.09 |
| I | 0.41 | 0.06 | 0.29 | 0.44 | 0.20 | 0.25 | 0.08 | 0.76 | 0.23 | 0.69 | 0.14 | 0.16 | 0.14 | 0.19 | 0.11 | 0.23 | 0.29 | 1.00 | 0.11 | 0.15 |
| A | 0.98 | 0.08 | 0.21 | 0.24 | 0.18 | 0.49 | 0.10 | 0.58 | 0.28 | 1.00 | 0.13 | 0.21 | 0.29 | 0.16 | 0.14 | 0.48 | 0.48 | 0.83 | 0.01 | 0.11 |
| Q | 1.00 | 0.14 | 0.72 | 0.73 | 0.10 | 0.72 | 0.30 | 0.16 | 0.85 | 0.32 | 0.08 | 0.50 | 0.39 | 0.58 | 0.53 | 0.79 | 0.55 | 0.33 | 0.03 | 0.11 |
| G | 0.52 | 0.04 | 0.15 | 0.14 | 0.04 | 1.00 | 0.04 | 0.05 | 0.13 | 0.10 | 0.02 | 0.14 | 0.16 | 0.07 | 0.05 | 0.37 | 0.22 | 0.14 | 0.01 | 0.02 |
| L | 0.23 | 0.04 | 0.06 | 0.08 | 0.49 | 0.12 | 0.05 | 0.57 | 0.17 | 1.00 | 0.24 | 0.07 | 0.08 | 0.07 | 0.09 | 0.13 | 0.18 | 0.64 | 0.06 | 0.21 |
| E | 0.74 | 0.13 | 0.91 | 0.90 | 0.16 | 1.00 | 0.28 | 0.32 | 0.72 | 0.32 | 0.09 | 0.58 | 0.29 | 0.48 | 0.43 | 0.68 | 0.50 | 0.44 | 0.02 | 0.23 |
| W | 0.17 | 0.04 | 0.13 | 0.13 | 0.51 | 0.10 | 0.20 | 0.11 | 0.19 | 0.37 | 0.05 | 0.12 | 0.10 | 0.11 | 0.20 | 0.16 | 0.11 | 0.13 | 1.00 | 0.54 |
| A | 1.00 | 0.18 | 0.28 | 0.29 | 0.09 | 0.61 | 0.15 | 0.23 | 0.32 | 0.41 | 0.10 | 0.29 | 0.30 | 0.17 | 0.18 | 0.54 | 0.46 | 0.56 | 0.01 | 0.07 |
| G | 1.00 | 0.10 | 0.22 | 0.26 | 0.22 | 0.59 | 0.12 | 0.60 | 0.37 | 0.71 | 0.15 | 0.22 | 0.61 | 0.20 | 0.24 | 0.57 | 0.59 | 0.93 | 0.01 | 0.18 |
| N | 0.89 | 0.08 | 1.00 | 0.94 | 0.10 | 0.88 | 0.46 | 0.18 | 0.86 | 0.31 | 0.07 | 0.72 | 0.38 | 0.76 | 0.50 | 0.80 | 0.59 | 0.31 | 0.02 | 0.09 |
| N | 0.73 | 0.06 | 1.00 | 0.77 | 0.11 | 0.80 | 0.58 | 0.17 | 0.73 | 0.43 | 0.07 | 0.74 | 0.34 | 0.51 | 0.32 | 0.66 | 0.44 | 0.27 | 0.22 | 0.10 |
| G | 0.44 | 0.05 | 0.27 | 0.24 | 0.09 | 1.00 | 0.39 | 0.10 | 0.53 | 0.20 | 0.04 | 0.31 | 0.28 | 0.23 | 0.37 | 0.40 | 0.27 | 0.21 | 0.02 | 0.12 |
| M | 0.85 | 0.12 | 0.24 | 0.25 | 0.25 | 0.69 | 0.13 | 0.62 | 0.53 | 0.67 | 0.26 | 0.26 | 0.38 | 0.19 | 0.28 | 0.48 | 0.46 | 1.00 | 0.01 | 0.10 |
| H | 0.64 | 0.11 | 1.00 | 0.74 | 0.10 | 0.55 | 0.46 | 0.17 | 0.63 | 0.25 | 0.06 | 0.51 | 0.28 | 0.39 | 0.46 | 0.54 | 0.38 | 0.36 | 0.02 | 0.08 |
| V | 0.34 | 0.05 | 0.08 | 0.10 | 0.10 | 0.16 | 0.04 | 0.61 | 0.12 | 0.47 | 0.09 | 0.07 | 0.10 | 0.06 | 0.06 | 0.16 | 0.23 | 1.00 | 0.00 | 0.05 |
| A | 0.58 | 0.10 | 0.13 | 0.17 | 0.50 | 0.28 | 0.14 | 0.94 | 0.22 | 0.87 | 0.14 | 0.16 | 0.17 | 0.12 | 0.12 | 0.30 | 0.37 | 1.00 | 0.02 | 0.93 |
| N | 0.72 | 0.16 | 0.55 | 0.38 | 0.10 | 0.69 | 0.29 | 0.16 | 0.62 | 0.20 | 0.05 | 0.76 | 0.32 | 0.22 | 0.23 | 1.00 | 0.61 | 0.24 | 0.02 | 0.10 |
| L | 0.45 | 0.21 | 0.15 | 0.15 | 0.19 | 0.23 | 0.08 | 0.30 | 0.34 | 1.00 | 0.56 | 0.16 | 0.15 | 0.12 | 0.12 | 0.26 | 0.26 | 0.44 | 0.01 | 0.09 |
| S | 0.67 | 0.17 | 0.22 | 0.20 | 0.07 | 0.56 | 0.09 | 0.09 | 0.32 | 0.12 | 0.04 | 0.29 | 0.33 | 0.12 | 0.16 | 1.00 | 0.54 | 0.18 | 0.03 | 0.05 |
| L | 0.10 | 0.01 | 0.03 | 0.04 | 0.17 | 0.06 | 0.04 | 0.14 | 0.07 | 1.00 | 0.08 | 0.04 | 0.05 | 0.05 | 0.08 | 0.08 | 0.07 | 0.20 | 0.71 | 0.10 |
| G | 0.25 | 0.01 | 0.10 | 0.09 | 0.03 | 1.00 | 0.02 | 0.02 | 0.07 | 0.04 | 0.01 | 0.09 | 0.07 | 0.04 | 0.02 | 0.19 | 0.10 | 0.07 | 0.00 | 0.02 |
| S | 0.46 | 0.07 | 0.17 | 0.18 | 0.07 | 1.00 | 0.07 | 0.08 | 0.17 | 0.12 | 0.02 | 0.16 | 0.53 | 0.11 | 0.08 | 0.40 | 0.24 | 0.18 | 0.01 | 0.05 |
| P | 0.62 | 0.06 | 0.39 | 0.35 | 0.13 | 1.00 | 0.13 | 0.13 | 0.31 | 0.17 | 0.04 | 0.29 | 0.54 | 0.17 | 0.16 | 0.53 | 0.40 | 0.26 | 0.01 | 0.13 |
| S | 0.82 | 0.16 | 0.77 | 0.62 | 0.27 | 1.00 | 0.21 | 0.25 | 0.63 | 0.43 | 0.09 | 0.47 | 0.36 | 0.32 | 0.31 | 0.75 | 0.55 | 0.43 | 0.03 | 0.55 |
| P | 0.69 | 0.08 | 0.74 | 0.58 | 0.16 | 1.00 | 0.22 | 0.17 | 0.39 | 0.27 | 0.06 | 0.44 | 0.39 | 0.26 | 0.15 | 0.71 | 0.43 | 0.32 | 0.06 | 0.14 |
| S | 0.71 | 0.09 | 0.37 | 0.35 | 0.10 | 1.00 | 0.20 | 0.15 | 0.39 | 0.24 | 0.08 | 0.32 | 0.35 | 0.20 | 0.27 | 0.71 | 0.46 | 0.35 | 0.02 | 0.12 |
| A | 0.99 | 0.12 | 0.75 | 0.73 | 0.21 | 0.73 | 0.35 | 0.23 | 1.00 | 0.45 | 0.09 | 0.52 | 0.78 | 0.51 | 0.65 | 0.90 | 0.77 | 0.46 | 0.10 | 0.27 |
| T | 0.87 | 0.07 | 0.27 | 0.32 | 0.17 | 0.52 | 0.12 | 0.30 | 0.37 | 1.00 | 0.14 | 0.24 | 0.47 | 0.19 | 0.18 | 0.56 | 0.61 | 0.53 | 0.05 | 0.08 |
| L | 0.45 | 0.07 | 0.13 | 0.18 | 0.32 | 0.29 | 0.07 | 0.32 | 0.17 | 1.00 | 0.14 | 0.12 | 0.16 | 0.12 | 0.08 | 0.26 | 0.32 | 0.51 | 0.04 | 0.14 |
| E | 0.64 | 0.06 | 0.59 | 0.67 | 0.11 | 0.49 | 0.42 | 0.17 | 1.00 | 0.41 | 0.12 | 0.50 | 0.30 | 0.60 | 0.64 | 0.57 | 0.45 | 0.30 | 0.03 | 0.14 |
| Q | 0.75 | 0.08 | 0.71 | 0.76 | 0.21 | 0.54 | 0.37 | 0.35 | 1.00 | 0.68 | 0.16 | 0.53 | 0.34 | 0.68 | 0.59 | 0.66 | 0.57 | 0.51 | 0.02 | 0.10 |
| A | 1.00 | 0.05 | 0.20 | 0.23 | 0.07 | 0.59 | 0.07 | 0.15 | 0.19 | 0.23 | 0.07 | 0.16 | 0.27 | 0.12 | 0.09 | 0.43 | 0.38 | 0.33 | 0.01 0.06 | |
| V | 0.50 | 0.13 | 0.13 | 0.14 | 0.56 | 0.27 | 0.08 | 0.53 | 0.20 | 0.77 | 0.16 | 0.13 | 0.16 | 0.10 | 0.13 | 0.30 | 0.31 | 1.00 | 0.05 | 0.17 |
| N | 0.90 | 0.09 | 1.00 | 0.94 | 0.56 | 0.66 | 0.35 | 0.43 | 0.98 | 0.59 | 0.16 | 0.71 | 0.34 | 0.50 | 0.51 | 0.70 | 0.54 | 0.59 | 0.10 | 0.46 |
| S | 0.73 | 0.11 | 0.56 | 0.55 | 0.45 | 0.57 | 0.47 | 0.20 | 1.00 | 0.58 | 0.09 | 0.54 | 0.32 | 0.43 | 0.91 | 0.63 | 0.46 | 0.33 | 0.17 | 0.94 |
| A | 1.00 | 0.06 | 0.21 | 0.23 | 0.15 | 0.99 | 0.07 | 0.17 | 0.19 | 0.45 | 0.07 | 0.18 | 0.28 | 0.12 | 0.08 | 0.48 | 0.39 | 0.35 | 0.04 | 0.11 |
| T | 0.54 | 0.10 | 0.18 | 0.21 | 0.49 | 0.28 | 0.14 | 0.56 | 0.39 | 0.60 | 0.14 | 0.21 | 0.19 | 0.18 | 0.24 | 0.38 | 0.52 | 1.00 | 0.19 | 0.65 |
| S | 0.87 | 0.08 | 0.73 | 0.77 | 0.11 | 0.65 | 0.34 | 0.19 | 1.00 | 0.32 | 0.08 | 0.61 | 0.36 | 0.51 | 0.59 | 0.77 | 0.64 | 0.31 | 0.03 | 0.12 |
| R | 0.73 | 0.09 | 0.45 | 0.55 | 0.10 | 0.50 | 0.35 | 0.15 | 1.00 | 0.35 | 0.10 | 0.42 | 0.39 | 0.50 | 0.86 | 0.68 | 0.45 | 0.28 | 0.09 | 0.10 |
| G | 0.26 | 0.01 | 0.13 | 0.11 | 0.02 | 1.00 | 0.04 | 0.02 | 0.09 | 0.04 | 0.01 | 0.11 | 0.07 | 0.05 | 0.03 | 0.20 | 0.10 | 0.07 | 0.00 | 0.01 |
| V | 0.56 | 0.12 | 0.15 | 0.15 | 0.16 | 0.33 | 0.07 | 0.54 | 0.24 | 0.51 | 0.10 | 0.17 | 0.19 | 0.10 | 0.12 | 0.44 | 0.40 | 1.00 | 0.01 | 0.07 |
| L | 0.26 | 0.04 | 0.07 | 0.08 | 0.24 | 0.13 | 0.08 | 0.46 | 0.13 | 1.00 | 0.12 | 0.08 | 0.10 | 0.07 | 0.07 | 0.17 | 0.23 | 0.66 | 0.01 | 0.08 |
| V | 0.40 | 0.08 | 0.11 | 0.12 | 1.00 | 0.22 | 0.10 | 0.57 | 0.18 | 0.82 | 0.13 | 0.14 | 0.16 | 0.10 | 0.10 | 0.28 | 0.31 | 0.95 | 0.06 | 0.55 |
| V | 0.42 | 0.10 | 0.09 | 0.11 | 0.08 | 0.21 | 0.04 | 0.43 | 0.13 | 0.48 | 0.09 | 0.08 | 0.12 | 0.07 | 0.07 | 0.20 | 0.24 | 1.00 | 0.00 | 0.04 |
| A | 0.71 | 0.38 | 0.15 | 0.17 | 0.50 | 0.40 | 0.10 | 0.39 | 0.21 | 0.46 | 0.08 | 0.16 | 0.22 | 0.12 | 0.20 | 0.42 | 0.37 | 0.76 | 1.00 | 0.18 |
| A | 1.00 | 0.06 | 0.19 | 0.21 | 0.05 | 0.49 | 0.07 | 0.10 | 0.18 | 0.14 | 0.03 | 0.17 | 0.29 | 0.11 | 0.08 | 0.51 | 0.40 | 0.25 | 0.01 | 0.04 |
| S | 1.00 | 0.08 | 0.20 | 0.22 | 0.06 | 0.52 | 0.07 | 0.13 | 0.21 | 0.18 | 0.04 | 0.20 | 0.30 | 0.12 | 0.10 | 0.59 | 0.45 | 0.28 | 0.01 | 0.05 |
| G | 0.24 | 0.01 | 0.09 | 0.08 | 0.02 | 1.00 | 0.02 | 0.02 | 0.07 | 0.04 | 0.01 | 0.08 | 0.06 | 0.03 | 0.02 | 0.18 | 0.09 | 0.06 | 0.00 | 0.01 |
| N | 0.51 | 0.05 | 0.75 | 0.47 | 0.08 | 0.55 | 0.40 | 0.12 | 0.70 | 0.18 | 0.04 | 1.00 | 0.21 | 0.25 | 0.18 | 0.64 | 0.43 | 0.17 | 0.01 | 0.11 |
| S | 0.54 | 0.05 | 0.64 | 0.63 | 0.09 | 1.00 | 0.15 | 0.08 | 0.30 | 0.11 | 0.03 | 0.35 | 0.19 | 0.25 | 0.10 | 0.49 | 0.30 | 0.15 | 0.01 | 0.09 |
| G | 0.32 | 0.02 | 0.19 | 0.15 | 0.04 | 1.00 | 0.06 | 0.04 | 0.15 | 0.07 | 0.01 | 0.19 | 0.10 | 0.07 | 0.04 | 0.27 | 0.15 | 0.10 | 0.00 | 0.05 |
| A | 0.99 | 0.12 | 0.57 | 0.59 | 0.15 | 0.75 | 0.35 | 0.27 | 0.85 | 0.69 | 0.12 | 0.48 | 1.00 | 0.54 | 0.87 | 0.93 | 0.66 | 0.47 | 0.04 | 0.13 |
| G | 0.76 | 0.16 | 0.57 | 0.55 | 0.43 | 1.00 | 0.31 | 0.18 | 0.53 | 0.40 | 0.07 | 0.48 | 0.34 | 0.39 | 0.36 | 0.74 | 0.51 | 0.31 | 0.03 | 0.44 |
| S | 1.00 | 0.21 | 0.39 | 0.45 | 0.28 | 0.70 | 0.16 | 0.29 | 0.56 | 0.34 | 0.11 | 0.39 | 0.50 | 0.23 | 0.20 | 0.92 | 0.98 | 0.52 | 0.02 | 0.24 |
| I | 0.37 | 1.00 | 0.13 | 0.13 | 0.40 | 0.31 | 0.14 | 0.38 | 0.20 | 0.57 | 0.10 | 0.15 | 0.18 | 0.11 | 0.16 | 0.31 | 0.25 | 0.66 | 0.02 | 0.35 |
| S | 0.69 | 0.08 | 0.67 | 0.49 | 0.28 | 1.00 | 0.19 | 0.15 | 0.37 | 0.29 | 0.06 | 0.41 | 0.24 | 0.20 | 0.14 | 0.61 | 0.50 | 0.28 | 0.02 | 0.40 |
| Y | 0.59 | 0.14 | 0.21 | 0.20 | 0.45 | 1.00 | 0.12 | 0.18 | 0.25 | 0.31 | 0.06 | 0.25 0.25 | 0.11 | 0.11 | 0.59 | 0.40 | 0.32 | 0.11 | 0.89 | |
| P | 0.32 | 0.05 | 0.07 | 0.10 | 0.18 | 0.16 | 0.10 | 0.05 | 0.12 | 0.13 | 0.02 | 0.09 | 1.00 | 0.11 | 0.09 | 0.25 | 0.15 | 0.11 | 0.01 | 0.47 |
| A | 1.00 | 0.06 | 0.27 | 0.27 | 0.06 | 0.82 | 0.09 | 0.12 | 0.28 | 0.16 | 0.04 | 0.25 | 0.30 | 0.14 | 0.10 | 0.57 | 0.58 | 0.28 | 0.01 | 0.05 |
| R | 1.00 | 0.18 | 0.54 | 0.45 | 0.19 | 0.86 | 0.28 | 0.25 | 0.91 | 0.39 | 0.10 | 0.60 | 0.40 | 0.32 | 0.64 | 0.98 | 0.72 | 0.48 | 0.04 | 0.18 |
| Y | 0.78 | 0.34 | 0.27 | 0.32 | 0.48 | 0.51 | 0.14 | 0.23 | 0.28 | 0.47 | 0.07 | 0.27 | 0.28 | 0.17 | 0.13 | 0.68 | 0.48 | 0.39 | 0.03 | 1.00 |
| A | 0.79 | 0.09 | 0.47 | 0.48 | 0.16 | 0.49 | 0.18 | 0.50 | 0.51 | 0.49 | 0.15 | 0.35 | 1.00 | 0.27 | 0.32 | 0.62 | 0.50 | 0.60 | 0.02 | 0.08 |
| N | 0.72 | 0.17 | 0.61 | 0.56 | 0.58 | 0.81 | 0.31 | 0.17 | 0.74 | 0.39 | 0.07 | 0.61 | 0.30 | 0.30 | 0.34 | 0.83 | 0.54 | 0.26 | 1.00 | 0.84 |
| A | 0.76 | 0.12 | 0.16 | 0.18 | 0.10 | 0.40 | 0.08 | 0.44 | 0.23 | 0.47 | 0.09 | 0.17 | 0.23 | 0.11 | 0.10 | 0.46 | 0.60 | 1.00 | 0.08 | 0.09 |
| M | 0.26 | 0.22 | 0.06 | 0.08 | 0.24 | 0.12 | 0.04 | 0.67 | 0.15 | 1.00 | 0.19 | 0.07 | 0.09 | 0.07 | 0.08 | 0.15 | 0.21 | 0.73 | 0.00 | 0.08 |
| A | 1.00 | 0.11 | 0.26 | 0.26 | 0.09 | 0.65 | 0.11 | 0.18 | 0.40 | 0.21 | 0.06 | 0.30 | 0.35 | 0.17 | 0.15 | 0.88 | 0.98 | 0.36 | 0.02 | 0.06 |
| V | 0.28 | 0.05 | 0.06 | 0.07 | 0.07 | 0.14 | 0.03 | 0.47 | 0.09 | 0.38 | 0.07 | 0.06 | 0.08 | 0.05 | 0.05 | 0.13 | 0.19 | 1.00 | 0.00 | 0.04 |
| G | 0.49 | 0.04 | 0.15 | 0.14 | 0.04 | 1.00 | 0.05 | 0.05 | 0.14 | 0.08 | 0.02 | 0.15 | 0.15 | 0.07 | 0.06 | 0.36 | 0.22 | 0.14 | 0.01 | 0.02 |
| A | 1.00 | 0.11 | 0.22 | 0.23 | 0.06 | 0.59 | 0.08 | 0.10 | 0.23 | 0.14 | 0.04 | 0.21 | 0.31 | 0.12 | 0.10 | 0.65 | 0.45 | 0.25 | 0.01 | 0.05 |
| T | 0.94 | 0.16 | 0.23 | 0.25 | 0.27 | 0.59 | 0.13 | 0.58 | 0.34 | 0.78 | 0.12 | 0.25 | 0.30 | 0.16 | 0.14 | 0.67 | 0.85 | 1.00 | 0.02 | 0.24 |
| D | 0.89 | 0.09 | 100 | 0.78 | 0.10 | 0.77 | 0.24 | 0.26 | 0.61 | 0.30 | 0.07 | 0.60 | 0.32 | 0.38 | 0.24 | 0.82 | 0.86 | 0.43 | 0.01 | 0.08 |
| Q | 0.97 | 0.13 | 0.70 | 0.83 | 0.42 | 0.74 | 0.40 | 0.40 | 1.00 | 0.80 | 0.15 | 0.56 | 0.55 | 0.75 | 0.58 | 0.98 | 0.72 | 0.60 | 0.11 | 0.21 |
| N | 1.00 | 0.11 | 0.87 | 0.81 | 0.30 | 0.86 | 0.36 | 0.21 | 0.86 | 0.32 | 0.11 | 0.76 | 0.48 | 0.45 | 0.35 | 0.97 | 0.70 | 0.34 | 0.03 | 0.30 |
| N | 0.41 | 0.03 | 0.45 | 0.34 | 0.08 | 1.00 | 0.13 | 0.07 | 0.28 | 0.14 | 0.02 | 0.31 | 0.22 | 0.15 | 0.11 | 0.35 | 0.22 | 0.14 | 0.09 | 0.13 |
| N | 0.75 | 0.08 | 0.54 | 0.54 | 0.21 | 0.57 | 0.38 | 0.28 | 1.00 | 0.68 | 0.14 | 0.54 | 0.33 | 0.47 | 0.67 | 0.67 | 0.62 | 0.46 | 0.03 | 0.21 |
| R | 0.43 | 0.08 | 0.15 | 0.17 | 0.16 | 0.25 | 0.25 | 0.56 | 0.82 | 0.87 | 0.19 | 0.20 | 0.22 | 0.24 | 1.00 | 0.38 | 0.38 | 0.83 | 0.04 | 0.07 |
| A | 1.00 | 0.12 | 0.21 | 0.23 | 0.09 | 0.54 | 0.10 | 0.12 | 0.24 | 0.20 | 0.04 | 0.21 | 0.72 | 0.14 | 0.12 | 0.65 | 0.45 | 0.28 | 0.01 | 0.14 |
| S | 0.78 | 0.14 | 0.49 | 0.43 | 0.41 | 0.62 | 0.21 | 0.16 | 0.47 | 0.34 | 0.06 | 0.40 | 0.52 | 0.23 | 0.39 | 0.92 | 0.58 | 0.30 | 1.00 | 0.50 |
| F | 0.06 | 0.03 | 0.02 | 0.02 | 1.00 | 0.03 | 0.05 | 0.08 | 0.03 | 0.20 | 0.02 | 0.04 | 0.03 | 0.02 | 0.03 | 0.06 | 0.04 | 0.06 | 0.10 | 0.64 |
| S | 0.73 | 0.14 | 0.27 | 0.24 | 0.09 | 0.61 | 0.10 | 0.11 | 0.34 | 0.31 | 0.05 | 0.31 | 0.35 | 0.14 | 0.16 | 1.00 | 0.58 | 0.22 | 0.03 | 0.06 |
| Q | 0.85 | 0.11 | 0.84 | 0.91 | 0.10 | 0.75 | 0.35 | 0.15 | 0.70 | 0.26 | 0.06 | 0.81 | 0.37 | 0.48 | 0.24 | 1.00 | 0.65 | 0.26 | 0.02 | 0.11 |
| Y | 0.39 | 0.09 | 0.18 | 0.22 | 0.54 | 0.25 | 0.20 | 0.16 | 0.50 | 0.27 | 0.06 | 0.19 | 0.19 | 0.17 | 0.51 | 0.34 | 0.27 | 0.29 | 0.29 | 1.00 |
| G | 0.28 | 0.58 | 0.12 | 0.10 | 0.03 | 1.00 | 0.04 | 0.04 | 0.09 | 0.05 | 0.01 | 0.11 | 0.08 | 0.04 | 0.03 | 0.24 | 0.12 | 0.09 | 0.00 | 0.04 |
| A | 0.99 | 0.23 | 0.35 | 0.34 | 0.11 | 0.75 | 0.20 | 0.16 | 0.56 | 0.31 | 0.08 | 0.38 | 1.00 | 0.24 | 0.40 | 1.00 | 0.68 | 0.35 | 0.03 | 0.10 |
| G | 1.00 | 0.90 | 0.56 | 0.66 | 0.20 | 0.99 | 0.23 | 0.32 | 0.58 | 0.44 | 0.09 | 0.45 | 0.40 | 0.39 | 0.37 | 0.98 | 0.69 | 0.72 | 0.21 | 0.21 |
| L | 0.53 | 0.07 | 0.13 | 0.15 | 0.17 | 0.26 | 0.09 | 0.51 | 0.22 | 0.91 | 0.13 | 0.15 | 0.52 | 0.13 | 0.11 | 0.36 | 0.49 | 1.00 | 0.04 | 0.12 |
| D | 0.49 | 0.19 | 1.00 | 0.81 | 0.16 | 0.45 | 0.19 | 0.17 | 0.36 | 0.83 | 0.11 | 0.43 | 0.18 | 0.30 | 0.11 | 0.39 | 0.30 | 0.26 | 0.05 | 0.08 |
| I | 0.54 | 0.09 | 0.11 | 0.14 | 0.23 | 0.25 | 0.06 | 0.71 | 0.17 | 0.87 | 0.12 | 0.11 | 0.16 | 0.09 | 0.09 | 0.25 | 0.32 | 1.00 | 0.01 | 0.11 |
| V | 0.87 | 0.16 | 0.21 | 0.22 | 1.00 | 0.49 | 0.12 | 0.44 | 0.35 | 0.69 | 0.22 | 0.25 | 0.30 | 0.14 | 0.16 | 0.70 | 0.72 | 0.89 | 0.03 | 0.39 |
| A | 1.00 | 0.06 | 0.19 | 0.21 | 0.10 | 0.53 | 0.07 | 0.12 | 0.21 | 0.16 | 0.04 | 0.18 | 0.28 | 0.11 | 0.08 | 0.50 | 0.56 | 0.28 | 0.01 | 0.05 |
| P | 0.30 | 0.05 | 0.06 | 0.09 | 0.22 | 0.13 | 0.08 | 0.05 | 0.11 | 0.12 | 0.02 | 0.08 | 1.00 | 0.09 | 0.08 | 0.23 | 0.14 | 0.10 | 0.01 | 0.33 |
| G | 0.32 | 0.03 | 0.12 | 0.10 | 0.03 | 1.00 | 0.03 | 0.03 | 0.10 | 0.05 | 0.01 | 0.11 | 0.10 | 0.05 | 0.04 | 0.29 | 0.15 | 0.08 | 0.01 | 0.02 |
| V | 0.89 | 0.12 | 0.38 | 0.43 | 0.12 | 1.00 | 0.17 | 0.34 | 0.40 | 0.47 | 0.09 | 0.35 | 0.33 | 0.31 | 0.17 | 0.81 | 0.65 | 0.86 | 0.02 | 0.11 |
| N | 0.75 | 0.08 | 0.70 | 0.63 | 0.17 | 1.00 | 0.27 | 0.14 | 0.76 | 0.33 | 0.09 | 0.58 | 0.29 | 0.41 | 0.29 | 0.69 | 0.45 | 0.25 | 0.07 | 0.31 |
| V | 0.29 | 0.05 | 0.07 | 0.09 | 0.19 | 0.13 | 0.04 | 0.91 | 0.15 | 0.59 | 0.12 | 0.08 | 0.09 | 0.06 | 0.08 | 0.15 | 0.24 | 1.00 | 0.03 | 0.08 |
| Q | 0.35 | 0.04 | 0.11 | 0.13 | 0.25 | 0.19 | 0.10 | 0.27 | 0.23 | 1.00 | 0.10 | 0.12 | 0.17 | 0.14 | 0.12 | 0.22 | 0.27 | 0.47 | 0.09 | 0.28 |
| S | 0.80 | 0.13 | 0.24 | 0.22 | 0.08 | 0.66 | 0.10 | 0.13 | 0.37 | 0.16 | 0.05 | 0.31 | 0.34 | 0.14 | 0.16 | 1.00 | 0.77 | 0.26 | 0.03 | 0.06 |
| T | 0.83 | 0.11 | 0.20 | 0.20 | 0.08 | 0.42 | 0.08 | 0.20 | 0.33 | 0.29 | 0.07 | 0.23 | 0.27 | 0.12 | 0.12 | 0.66 | 1.00 | 0.40 | 0.01 | 0.06 |
| Y | 0.30 | 0.05 | 0.39 | 0.29 | 0.24 | 0.26 | 0.13 | 0.21 | 0.23 | 0.44 | 0.05 | 0.21 | 0.16 | 0.14 | 0.21 | 0.26 | 0.20 | 0.35 | 1.00 | 0.41 |
| P | 0.46 | 0.06 | 0.19 | 0.19 | 0.23 | 0.29 | 0.14 | 0.41 | 0.27 | 1.00 | 0.11 | 0.20 | 0.63 | 0.17 | 0.14 | 0.35 | 0.33 | 0.52 | 0.05 | 0.20 |
| G | 0.52 | 0.09 | 0.42 | 0.36 | 0.09 | 1.00 | 0.30 | 0.12 | 0.41 | 0.25 | 0.05 | 0.38 | 0.31 | 0.28 | 0.26 | 0.49 | 0.36 | 0.22 | 0.05 | 0.11 |
| S | 0.66 | 0.08 | 0.47 | 0.41 | 0.10 | 1.00 | 0.20 | 0.15 | 0.63 | 0.19 | 0.05 | 0.45 | 0.36 | 0.27 | 0.33 | 0.68 | 0.52 | 0.25 | 0.02 | 0.15 |
| T | 0.55 | 1.00 | 0.24 | 0.24 | 0.11 | 0.79 | 0.13 | 0.11 | 0.40 | 0.16 | 0.04 | 0.22 | 0.20 | 0.16 | 0.28 | 0.48 | 0.35 | 0.21 | 0.05 | 0.21 |
| Y | 0.34 | 0.10 | 0.10 | 0.10 | 0.51 | 0.17 | 0.10 | 0.13 | 0.19 | 0.26 | 0.05 | 0.15 | 0.13 | 0.08 | 0.07 | 0.32 | 0.52 | 0.21 | 0.05 | 1.00 |
| A | 1.00 | 0.13 | 0.65 | 0.60 | 0.12 | 0.96 | 0.31 | 0.25 | 0.70 | 0.57 | 0.12 | 0.59 | 0.35 | 0.42 | 0.36 | 0.74 | 0.68 | 0.46 | 0.02 | 0.09 |
| S | 0.91 | 0.13 | 0.36 | 0.39 | 0.33 | 0.66 | 0.18 | 0.40 | 0.74 | 0.66 | 0.19 | 0.40 | 0.35 | 0.27 | 0.92 | 1.00 | 0.59 | 0.03 | 0.41 | |
| L | 0.28 | 0.05 | 0.15 | 0.17 | 0.45 | 0.20 | 0.52 | 0.48 | 0.33 | 1.00 | 0.25 | 0.19 | 0.14 | 0.24 | 0.18 | 0.21 | 0.21 | 0.52 | 0.05 | 0.31 |
| N | 0.79 | 0.13 | 0.37 | 0.31 | 0.13 | 0.74 | 0.17 | 0.15 | 0.47 | 0.20 | 0.06 | 0.44 | 0.34 | 0.22 | 0.19 | 1.00 | 0.75 | 0.26 | 0.03 | 0.11 |
| G | 0.24 | 0.01 | 0.09 | 0.08 | 0.02 | 1.00 | 0.02 | 0.02 | 0.06 | 0.04 | 0.01 | 0.08 | 0.06 | 0.03 | 0.02 | 0.18 | 0.09 | 0.06 | 0.00 | 0.01 |
| T | 0.54 | 0.06 | 0.15 | 0.13 | 0.06 | 0.26 | 0.07 | 0.15 | 0.29 | 0.15 | 0.05 | 0.19 | 0.18 | 0.09 | 0.09 | 0.51 | 1.00 | 0.26 | 0.01 | 0.04 |
| S | 0.66 | 0.14 | 0.22 | 0.19 | 0.07 | 0.56 | 0.09 | 0.09 | 0.32 | 0.12 | 0.04 | 0.29 | 0.32 | 0.12 | 0.16 | 1.00 | 0.53 | 0.17 | 0.03 | 0.05 |
| M | 0.54 | 0.04 | 0.12 | 0.15 | 0.25 | 0.26 | 0.07 | 0.31 | 0.42 | 1.00 | 0.83 | 0.12 | 0.17 | 0.15 | 0.15 | 0.29 | 0.30 | 0.52 | 0.01 | 0.07 |
| A | 1.00 | 0.07 | 0.19 | 0.21 | 0.05 | 0.51 | 0.07 | 0.10 | 0.19 | 0.14 | 0.04 | 0.18 | 0.29 | 0.11 | 0.09 | 0.54 | 0.41 | 0.25 | 0.01 | 0.04 |
| T | 1.00 | 0.33 | 0.22 | 0.23 | 0.08 | 0.54 | 0.09 | 0.17 | 0.32 | 0.22 | 0.06 | 0.25 | 0.32 | 0.13 | 0.12 | 0.74 | 0.89 | 0.35 | 0.01 | 0.06 |
| P | 0.29 | 0.03 | 0.06 | 0.08 | 0.01 | 0.14 | 0.06 | 0.03 | 0.10 | 0.07 | 0.01 | 0.07 | 1.00 | 0.09 | 0.07 | 0.22 | 0.13 | 0.08 | 0.00 | 0.01 |
| H | 0.20 | 0.04 | 0.18 | 0.20 | 0.22 | 0.15 | 1.00 | 0.13 | 0.24 | 0.66 | 0.08 | 0.25 | 0.15 | 0.31 | 0.19 | 0.17 | 0.13 | 0.23 | 0.01 | 0.19 |
| V | 0.57 | 0.06 | 0.10 | 0.13 | 0.09 | 0.27 | 0.05 | 0.42 | 0.13 | 0.41 | 0.08 | 0.10 | 0.16 | 0.08 | 0.06 | 0.24 | 0.29 | 1.00 | 0.00 | 0.05 |
| A | 1.00 | 0.10 | 0.19 | 0.21 | 0.07 | 0.50 | 0.07 | 0.14 | 0.21 | 0.18 | 0.04 | 0.19 | 0.29 | 0.11 | 0.09 | 0.55 | 0.51 | 0.36 | 0.01 | 0.07 |
| G | 0.28 | 0.01 | 0.10 | 0.09 | 0.02 | 1.00 | 0.02 | 0.02 | 0.07 | 0.04 | 0.01 | 0.09 | 0.07 | 0.04 | 0.02 | 0.19 | 0.10 | 0.07 | 0.00 | 0.01 |
| A | 0.61 | 0.10 | 0.13 | 0.15 | 0.15 | 0.34 | 0.07 | 0.55 | 0.19 | 1.00 | 0.15 | 0.13 | 0.19 | 0.10 | 0.09 | 0.31 | 0.34 | 0.97 | 0.01 | 0.09 |
| A | 1.00 | 0.16 | 0.20 | 0.24 | 0.46 | 0.55 | 0.10 | 0.42 | 0.24 | 0.79 | 0.12 | 0.19 | 0.29 | 0.15 | 0.13 | 0.49 | 0.46 | 0.74 | 0.01 | 0.23 |
| A | 1.00 | 0.06 | 0.18 | 0.21 | 0.07 | 0.56 | 0.07 | 0.14 | 0.18 | 0.31 | 0.06 | 0.16 | 0.28 | 0.11 | 0.08 | 0.46 | 0.42 | 0.32 | 0.01 | 0.04 |
| L | 0.11 | 0.03 | 0.02 | 0.04 | 0.18 | 0.05 | 0.03 | 0.14 | 0.07 | 1.00 | 0.08 | 0.04 | 0.05 | 0.04 | 0.03 | 0.06 | 0.07 | 0.21 | 0.01 | 0.25 |
| V | 0.36 | 0.04 | 0.08 | 0.10 | 0.25 | 0.17 | 0.05 | 0.38 | 0.13 | 1.00 | 0.14 | 0.08 | 0.12 | 0.08 | 0.06 | 0.18 | 0.22 | 0.58 | 0.01 | 0.21 |
| K | 0.20 | 0.02 | 0.07 | 0.09 | 0.14 | 0.12 | 0.06 | 0.23 | 0.36 | 1.00 | 0.14 | 0.08 | 0.09 | 0.12 | 0.17 | 0.13 | 0.14 | 0.30 | 0.09 | 0.05 |
| Q | 1.00 | 0.11 | 0.70 | 0.98 | 0.09 | 0.86 | 0.28 | 0.16 | 0.56 | 0.28 | 0.07 | 0.44 | 0.45 | 0.71 | 0.25 | 0.94 | 0.60 | 0.31 | 0.02 | 0.07 |
| K | 0.90 | 0.08 | 0.28 | 0.36 | 0.22 | 0.62 | 0.21 | 0.30 | 0.80 | 1.00 | 0.14 | 0.27 | 0.32 | 0.37 | 0.40 | 0.51 | 0.48 | 0.55 | 0.07 | 0.31 |
| N | 0.90 | 0.11 | 0.81 | 0.75 | 0.70 | 1.00 | 0.85 | 0.27 | 0.88 | 0.72 | 0.10 | 0.90 | 0.45 | 0.57 | 0.48 | 0.81 | 0.57 | 0.44 | 0.04 | 0.75 |
| P | 0.42 | 0.04 | 0.20 | 0.23 | 0.04 | 0.34 | 0.13 | 0.08 | 0.39 | 0.19 | 0.03 | 0.19 | 1.00 | 0.20 | 0.27 | 0.35 | 0.22 | 0.18 | 0.01 | 0.03 |
| S | 0.98 | 0.19 | 0.82 | 0.76 | 0.37 | 0.81 | 0.40 | 0.28 | 1.00 | 0.92 | 0.12 | 0.73 | 0.40 | 0.49 | 0.35 | 0.97 | 0.84 | 0.48 | 0.03 | 0.46 |
| W | 0.24 | 0.07 | 0.08 | 0.10 | 0.14 | 0.14 | 0.05 | 0.16 | 0.13 | 1.00 | 0.10 | 0.08 | 0.16 | 0.08 | 0.09 | 0.17 | 40.15 | 0.24 | 0.32 | 0.13 |
| S | 0.74 | 0.10 | 0.31 | 0.28 | 0.08 | 0.54 | 0.12 | 0.16 | 0.47 | 0.24 | 0.06 | 0.33 | 0.31 | 0.17 | 0.21 | 0.84 | 1.00 | 0.29 | 0.02 | 0.06 |
| N | 0.55 | 0.05 | 0.21 | 0.22 | 0.08 | 0.36 | 0.13 | 0.11 | 0.24 | 0.22 | 0.03 | 0.22 | 0.89 | 0.15 | 0.19 | 0.42 | 0.28 | 0.26 | 1.00 | 0.06 |
| V | 1.00 | 0.11 | 0.48 | 0.49 | 0.13 | 0.64 | 0.28 | 0.25 | 0.80 | 0.34 | 0.08 | 0.39 | 0.48 | 0.38 | 0.91 | 0.79 | 0.61 | 0.52 | 0.04 | 0.15 |
| Q | 0.81 | 0.05 | 0.94 | 1.00 | 0.15 | 0.65 | 0.31 | 0.19 | 0.55 | 0.41 | 0.12 | 0.46 | 0.32 | 0.76 | 0.24 | 0.56 | 0.46 | 0.31 | 0.01 | 0.08 |
| I | 0.38 | 0.05 | 0.09 | 0.11 | 0.15 | 0.18 | 0.05 | 0.60 | 0.17 | 1.00 | 0.21 | 0.09 | 0.13 | 0.08 | 0.08 | 0.18 | 0.24 | 0.95 | 0.00 | 0.06 |
| R | 0.44 | 0.34 | 0.27 | 0.40 | 0.18 | 0.28 | 0.29 | 0.17 | 0.94 | 0.35 | 0.08 | 0.23 | 0.26 | 0.59 | 1.00 | 0.41 | 0.33 | 0.26 | 0.04 | 0.13 |
| N | 1.00 | 0.11 | 0.80 | 0.78 | 0.21 | 0.75 | 0.90 | 0.19 | 0.91 | 0.32 | 0.09 | 0.64 | 0.42 | 0.64 | 0.54 | 0.82 | 0.63 | 0.35 | 0.03 | 0.38 |
| H | 0.52 | 0.06 | 0.18 | 0.24 | 0.22 | 0.27 | 0.21 | 0.43 | 0.43 | 1.00 | 0.12 | 0.17 | 0.21 | 0.24 | 0.42 | 0.32 | 0.32 | 0.59 | 0.02 | 0.16 |
| L | 0.20 | 0.03 | 0.05 | 0.07 | 0.15 | 0.10 | 0.04 | 0.37 | 0.11 | 1.00 | 0.13 | 0.06 | 0.08 | 0.06 | 0.06 | 0.12 | 0.18 | 0.47 | 0.00 | 0.05 |
| K | 0.60 | 0.08 | 0.33 | 0.44 | 0.19 | 0.37 | 0.17 | 0.56 | 0.94 | 0.90 | 0.27 | 0.29 | 0.24 | 0.34 | 0.36 | 0.45 | 0.48 | 1.00 | 0.01 | 0.12 |
| N | 0.74 | 0.10 | 0.51 | 0.55 | 0.24 | 0.55 | 0.41 | 0.21 | 1.00 | 0.59 | 0.14 | 0.52 | 0.34 | 0.50 | 0.66 | 0.74 | 0.65 | 0.35 | 0.20 | 0.33 |
| T | 0.66 | 0.08 | 0.22 | 0.20 | 0.15 | 0.40 | 0.14 | 0.20 | 0.49 | 0.48 | 0.09 | 0.29 | 0.25 | 0.15 | 0.28 | 0.65 | 1.00 | 0.33 | 0.02 | 0.15 |
| A | 1.00 | 0.10 | 0.22 | 0.22 | 0.06 | 0.57 | 0.08 | 0.12 | 0.22 | 0.16 | 0.04 | 0.21 | 0.30 | 0.12 | 0.09 | 0.59 | 0.48 | 0.28 | 0.01 | 0.05 |
| T | 0.80 | 0.10 | 0.51 | 0.51 | 0.20 | 0.50 | 0.26 | 0.39 | 0.96 | 0.54 | 0.12 | 0.43 | 0.36 | 0.37 | 0.61 | 0.77 | 1.00 | 0.67 | 0.03 | 0.21 |
| S | 0.75 | 0.10 | 0.56 | 0.53 | 0.21 | 0.55 | 0.28 | 0.20 | 1.00 | 0.41 | 0.11 | 0.43 | 0.97 | 0.38 | 0.69 | 0.70 | 0.52 | 0.36 | 0.14 | 0.30 |
| L | 0.38 | 0.03 | 0.24 | 0.21 | 0.26 | 0.50 | 0.09 | 0.30 | 0.25 | 1.00 | 0.14 | 0.20 | 0.13 | 0.12 | 0.11 | 0.26 | 0.24 | 0.42 | 0.01 | 0.11 |
| G | 0.61 | 0.06 | 0.46 | 0.40 | 0.10 | 1.00 | 0.23 | 0.18 | 0.43 | 0.25 | 0.05 | 0.38 | 0.48 | 0.25 | 0.22 | 0.52 | 0.34 | 0.39 | 0.05 | 0.14 |
| S | 0.84 | 0.23 | 0.47 | 0.41 | 0.52 | 0.55 | 0.25 | 0.55 | 0.54 | 0.61 | 0.11 | 0.44 | 0.48 | 0.24 | 0.30 | 0.73 | 0.65 | 1.00 | 0.03 | 0.37 |
| T | 0.96 | 0.13 | 0.62 | 0.53 | 0.12 | 1.00 | 0.28 | 0.28 | 0.67 | 0.48 | 0.10 | 0.54 | 0.89 | 0.32 | 0.33 | 0.98 | 0.64 | 0.51 | 0.02 | 0.08 |
| N | 0.74 | 0.12 | 0.45 | 0.42 | 1.00 | 0.60 | 0.89 | 0.29 | 0.85 | 0.78 | 0.11 | 0.55 | 0.47 | 0.46 | 0.64 | 0.78 | 0.61 | 0.44 | 0.24 | 0.47 |
| L | 0.69 | 0.08 | 0.43 | 0.42 | 0.54 | 1.00 | 0.28 | 0.23 | 0.54 | 0.77 | 0.12 | 0.33 | 0.27 | 0.32 | 0.39 | 0.57 | 0.41 | 0.37 | 0.03 | 0.53 |
| Y | 0.36 | 0.15 | 0.15 | 0.20 | 1.00 | 0.22 | 0.20 | 0.18 | 0.26 | 0.41 | 0.07 | 0.17 | 0.16 | 0.28 | 0.13 | 0.36 | 0.34 | 0.26 | 0.07 | 0.99 |
| G | 0.30 | 0.02 | 0.11 | 0.10 | 0.03 | 1.00 | 0.03 | 0.05 | 0.11 | 0.09 | 0.02 | 0.10 | 0.22 | 0.06 | 0.05 | 0.22 | 0.12 | 0.13 | 0.00 | 0.01 |
| S | 0.61 | 0.10 | 0.39 | 0.30 | 0.90 | 0.44 | 0.43 | 0.17 | 0.58 | 0.33 | 0.06 | 0.53 | 0.24 | 0.23 | 0.23 | 0.59 | 0.42 | 0.25 | 0.48 | 1.00 |
| G | 0.27 | 0.02 | 0.11 | 0.10 | 0.04 | 1.00 | 0.03 | 0.05 | 0.13 | 0.15 | 0.02 | 0.09 | 0.08 | 0.05 | 0.05 | 0.20 | 0.11 | 0.12 | 0.00 | 0.05 |
| L | 0.18 | 0.02 | 0.07 | 0.09 | 0.14 | 0.10 | 0.10 | 0.22 | 0.21 | 1.00 | 0.11 | 0.07 | 0.08 | 0.09 | 0.17 | 0.12 | 0.12 | 0.33 | 0.01 | 0.07 |
| V | 0.38 | 0.04 | 0.11 | 0.12 | 0.14 | 0.18 | 0.06 | 0.48 | 0.16 | 1.00 | 0.18 | 0.09 | 0.12 | 0.09 | 0.07 | 0.18 | 0.22 | 0.78 | 0.00 | 0.05 |
| N | 0.52 | 0.07 | 1.00 | 0.71 | 0.49 | 0.50 | 0.34 | 0.15 | 0.67 | 0.23 | 0.05 | 0.69 | 0.21 | 0.36 | 0.29 | 0.52 | 0.38 | 0.21 | 0.03 | 0.81 |
| A | 1.00 | 0.08 | 0.28 | 0.27 | 0.13 | 0.54 | 0.14 | 0.30 | 0.31 | 0.62 | 0.10 | 0.31 | 0.44 | 0.16 | 0.12 | 0.55 | 0.47 | 0.62 | 0.01 | 0.13 |
| E | 0.59 | 0.06 | 0.36 | 0.40 | 0.21 | 1.00 | 0.15 | 0.15 | 0.47 | 0.44 | 0.08 | 0.29 | 0.22 | 0.22 | 0.19 | 0.47 | 0.36 | 0.28 | 0.09 | 0.28 |
| A | 1.00 | 0.08 | 0.42 | 0.43 | 0.16 | 0.72 | 0.20 | 0.17 | 0.83 | 0.33 | 0.07 | 0.40 | 0.34 | 0.28 | 0.51 | 0.64 | 0.52 | 0.35 | 0.03 | 0.20 |
| A | 1.00 | 0.06 | 0.26 | 0.28 | 0.24 | 0.63 | 0.11 | 0.25 | 0.33 | 0.70 | 0.28 | 0.21 | 0.34 | 0.20 | 0.14 | 0.51 | 0.44 | 0.45 | 0.09 | 0.09 |
| T | 0.50 | 0.07 | 0.17 | 0.20 | 0.16 | 0.39 | 0.08 | 0.45 | 0.22 | 0.57 | 0.09 | 0.14 | 0.16 | 0.14 | 0.09 | 0.29 | 0.33 | 1.00 | 0.04 | 0.18 |
| R | 0.73 | 0.07 | 0.76 | 0.77 | 0.19 | 0.60 | 0.37 | 0.21 | 1.00 | 0.41 | 0.12 | 0.56 | 0.38 | 0.59 | 0.46 | 0.64 | 0.59 | 0.39 | 0.02 | 0.14 |

TABLE 3

| | GG36 | CB min | Cbmin*m + b | | | | | | | | | | | |
|---|---|---|---|---|---|---|---|---|---|---|---|---|---|---|
| 1 | A | 29.79 | 4.218 | | | | | | | | | | | |
| 2 | Q | 26.66 | 3.732 | | | | | | | | | | | |
| 3 | S | 23.77 | 3.284 | | | | | | | | | | | |
| 4 | V | 23.79 | 3.287 | | | | | | | | | | | |
| 5 | P | 18.20 | 2.421 | | | | | | | | | | | |
| 6 | W | 19.66 | 2.647 | | | | | | | | | | | |
| 7 | G | 16.88 | 2.216 | | | | | | | | | | | |
| 8 | I | 19.39 | 2.605 | | | | | | | | | | | |
| 9 | S | 22.76 | 3.127 | | | | | | | | | | | |
| 10 | R | 19.29 | 2.590 | | | | | | | | | | | |
| 11 | V | 18.36 | 2.445 | | | | | | | | | | | |
| 12 | Q | 23.87 | 3.300 | | | | | | | | | | | |
| 13 | A | 21.38 | 2.915 | | | | | | | | | | | |
| 14 | P | 25.94 | 3.621 | | | | | | | | | | | |
| 15 | A | 27.85 | 3.916 | | | | | | | | | | | |
| 16 | A | 25.91 | 3.617 | | | | | | | | | | | |
| 17 | H | 27.07 | 3.796 | | | | | | | | | | | |
| 18 | N | 31.40 | 4.467 | | | | | | | | | | | |
| 19 | R | 31.41 | 4.469 | | | | | | | | | | | |
| 20 | G | 31.23 | 4.441 | | | | | | | | | | | |
| 21 | L | 27.66 | 3.887 | | | | | | | | | | | |
| 22 | T | 26.48 | 3.704 | | | | | | | | | | | |
| 23 | G | 23.93 | 3.309 | | | | | | | | | | | |
| 24 | S | 28.47 | 4.013 | | | | | | | | | | | |
| 25 | G | 27.13 | 3.806 | | | | | | | | | | | |
| 26 | V | 23.32 | 3.215 | | | | | | | | | | | |
| 27 | K | 22.40 | 3.072 | | | | | | | | | | | |
| 28 | V | 17.70 | 2.343 | | | | | | | | | | | |
| 29 | A | 15.74 | 2.040 | | | | | | | | | | | |
| 30 | V | 11.71 | 1.415 | | | | | | | | | | | |
| 31 | L | 9.83 | 1.124 | | | | | | | | | | | |
| 32 | D | 7.03 | 0.690 | | | D | | | | | | | | |
| 33 | T | 6.58 | 0.621 | A | | | S | T | | | | | | |
| 34 | G | 10.71 | 1.260 | | | | | | | | | | | |
| 35 | I | 13.43 | 1.682 | | | | | | | | | | | |
| 36 | S | 15.03 | 1.929 | | | | | | | | | | | |
| 37 | T | 19.87 | 2.680 | | | | | | | | | | | |
| 38 | H | 18.22 | 2.424 | | | | | | | | | | | |
| 39 | P | 23.54 | 3.249 | | | | | | | | | | | |
| 40 | D | 21.01 | 2.856 | | | | | | | | | | | |
| 41 | L | 18.25 | 2.429 | | | | | | | | | | | |
| 42 | N | 22.75 | 3.127 | | | | | | | | | | | |
| 43 | I | 18.66 | 2.493 | | | | | | | | | | | |
| 44 | R | 22.22 | 3.044 | | | | | | | | | | | |
| 45 | G | 20.58 | 2.790 | | | | | | | | | | | |
| 46 | G | 18.02 | 2.393 | | | | | | | | | | | |
| 47 | A | 17.27 | 2.277 | | | | | | | | | | | |
| 48 | S | 15.44 | 1.993 | | | | | | | | | | | |
| 49 | F | 12.05 | 1.467 | | | | | | | | | | | |
| 50 | V | 11.61 | 1.399 | | | | | | | | | | | |
| 51 | P | 14.93 | 1.913 | | | | | | | | | | | |
| 52 | G | 17.28 | 2.279 | | | | | | | | | | | |
| 53 | E | 14.46 | 1.841 | | | | | | | | | | | |
| 54 | P | 19.76 | 2.663 | | | | | | | | | | | |
| 55 | S | 17.59 | 2.327 | | | | | | | | | | | |
| 56 | T | 15.89 | 2.062 | | | | | | | | | | | |
| 57 | Q | 15.95 | 2.072 | | | | | | | | | | | |
| 58 | D | 11.11 | 1.322 | | | | | | | | | | | |
| 59 | G | 11.86 | 1.438 | | | | | | | | | | | |
| 60 | N | 7.41 | 0.749 | A | | D | G | K | N | S | | | | |
| 61 | G | 9.19 | 1.024 | | | | | | | | | | | |
| 62 | H | 4.56 | 0.307 | | | | | | | H | | | | |
| 63 | G | 7.83 | 0.813 | | | | G | | | | | | | |
| 64 | T | 11.86 | 1.438 | | | | | | | | | | | |
| 65 | H | 9.84 | 1.126 | | | | | | | | | | | |
| 66 | V | 8.55 | 0.926 | | C | | | | | | | | | |
| 67 | A | 12.95 | 1.607 | | | | | | | | | | | |
| 68 | G | 15.05 | 1.933 | | | | | | | | | | | |
| 69 | T | 13.08 | 1.627 | | | | | | | | | | | |
| 70 | I | 15.30 | 1.972 | | | | | | | | | | | |
| 71 | A | 18.53 | 2.473 | | | | | | | | | | | |
| 72 | A | 18.96 | 2.539 | | | | | | | | | | | |
| 73 | L | 23.52 | 3.245 | | | | | | | | | | | |
| 74 | N | 26.48 | 3.704 | | | | | | | | | | | |
| 75 | N | 27.50 | 3.862 | | | | | | | | | | | |
| 76 | S | 30.50 | 4.328 | | | | | | | | | | | |
| 77 | I | 25.89 | 3.614 | | | | | | | | | | | |
| 78 | G | 22.63 | 3.108 | | | | | | | | | | | |
| 79 | V | 17.36 | 2.292 | | | | | | | | | | | |
| 80 | L | 20.84 | 2.830 | | | | | | | | | | | |
| 81 | G | 18.07 | 2.401 | | | | | | | | | | | |
| 82 | V | 18.08 | 2.403 | | | | | | | | | | | |
| 83 | A | 20.47 | 2.773 | | | | | | | | | | | |
| 84 | P | 22.98 | 3.161 | | | | | | | | | | | |
| 85 | S | 26.02 | 3.633 | | | | | | | | | | | |
| 86 | A | 20.70 | 2.808 | | | | | | | | | | | |
| 87 | E | 22.82 | 3.137 | | | | | | | | | | | |
| 88 | L | 17.99 | 2.388 | | | | | | | | | | | |
| 89 | Y | 17.79 | 2.358 | | | | | | | | | | | |
| 90 | A | 14.48 | 1.844 | | | | | | | | | | | |
| 91 | V | 13.45 | 1.685 | | | | | | | | | | | |
| 92 | K | 11.89 | 1.443 | | | | | | | | | | | |
| 93 | V | 7.87 | 0.819 | V | | | | | | | | | | |
| 94 | L | 5.94 | 0.520 | | | | | | | | | | | L |
| 95 | G | 9.34 | 1.048 | | | | | | | | | | | |
| 96 | A | 10.83 | 1.278 | | | | | | | | | | | |
| 97 | S | 8.91 | 0.981 | | | C | | | | | | | | |
| 98 | G | 4.98 | 0.371 | | | G | | | | | | | | |
| 99 | S | 5.48 | 0.450 | A | G | K | S | T | | | | | | |
| 100 | G | 5.14 | 0.397 | A | G | | | | | | | | | |
| 101 | S | 7.34 | 0.737 | A | S | T | | | | | | | | |
| 102 | V | 6.71 | 0.640 | A | | D | E | G | L | S | T | V | Y | |
| 103 | S | 10.41 | 1.214 | | | | | | | | | | | |
| 104 | S | 8.74 | 0.954 | G | | | | | | | | | | |
| 105 | I | 5.63 | 0.473 | I | L | V | | | | | | | | |
| 106 | A | 10.33 | 1.202 | | | | | | | | | | | |
| 107 | Q | 12.52 | 1.541 | | | | | | | | | | | |
| 108 | G | 11.68 | 1.411 | | | | | | | | | | | |
| 109 | L | 11.87 | 1.440 | | | | | | | | | | | |
| 110 | E | 15.52 | 2.006 | | | | | | | | | | | |
| 111 | W | 16.01 | 2.082 | | | | | | | | | | | |
| 112 | A | 15.72 | 2.036 | | | | | | | | | | | |
| 113 | G | 18.84 | 2.520 | | | | | | | | | | | |
| 114 | N | 20.61 | 2.794 | | | | | | | | | | | |
| 115 | N | 21.16 | 2.879 | | | | | | | | | | | |
| 116 | G | 22.85 | 3.142 | | | | | | | | | | | |
| 117 | M | 18.86 | 2.523 | | | | | | | | | | | |
| 118 | H | 22.17 | 3.036 | | | | | | | | | | | |
| 119 | V | 17.56 | 2.322 | | | | | | | | | | | |
| 120 | A | 14.02 | 1.772 | | | | | | | | | | | |
| 121 | N | 11.59 | 1.396 | | | | | | | | | | | |
| 122 | L | 8.78 | 0.960 | L | | | | | | | | | | |
| 123 | S | 5.62 | 0.471 | A | G | S | T | | | | | | | |
| 124 | L | 5.04 | 0.381 | L | W | | | | | | | | | |
| 125 | G | 4.70 | 0.328 | G | | | | | | | | | | |
| 126 | S | 4.80 | 0.345 | A | G | P | S | | | | | | | |
| 127 | P | 9.44 | 1.063 | | | | | | | | | | | |
| 128 | S | 9.95 | 1.142 | | | | | | | | | | | |
| 129 | P | 11.67 | 1.409 | | | | | | | | | | | |
| 130 | S | 8.65 | 0.940 | G | | | | | | | | | | |
| 131 | A | 14.35 | 1.824 | | | | | | | | | | | |
| 132 | T | 11.20 | 1.336 | | | | | | | | | | | |
| 133 | L | 8.21 | 0.873 | L | | | | | | | | | | |
| 134 | E | 13.16 | 1.640 | | | | | | | | | | | |
| 135 | Q | 14.88 | 1.906 | | | | | | | | | | | |
| 136 | A | 12.02 | 1.464 | | | | | | | | | | | |
| 137 | V | 12.55 | 1.545 | | | | | | | | | | | |
| 138 | N | 17.07 | 2.245 | | | | | | | | | | | |
| 139 | S | 17.36 | 2.290 | | | | | | | | | | | |
| 140 | A | 15.61 | 2.019 | | | | | | | | | | | |
| 141 | T | 18.34 | 2.443 | | | | | | | | | | | |
| 142 | S | 21.93 | 2.999 | | | | | | | | | | | |
| 143 | R | 21.23 | 2.891 | | | | | | | | | | | |
| 144 | G | 22.33 | 3.060 | | | | | | | | | | | |
| 145 | V | 17.90 | 2.374 | | | | | | | | | | | |
| 146 | L | 18.43 | 2.457 | | | | | | | | | | | |
| 147 | V | 13.94 | 1.761 | | | | | | | | | | | |
| 148 | V | 12.28 | 1.503 | | | | | | | | | | | |
| 149 | A | 9.22 | 1.030 | | | | | | | | | | | |
| 150 | A | 4.22 | 0.254 | A | G | P | S | T | | | | | | |
| 151 | S | 8.11 | 0.857 | A | | | | | | | | | | |
| 152 | G | 4.68 | 0.326 | G | | | | | | | | | | |
| 153 | N | 5.10 | 0.391 | A | D | E | G | H | K | N | S | T | | |
| 154 | S | 9.44 | 1.064 | | | | | | | | | | | |
| 155 | G | 11.06 | 1.314 | | | | | | | | | | | |
| 156 | A | 12.87 | 1.595 | | | | | | | | | | | |
| 157 | G | 15.00 | 1.925 | | | | | | | | | | | |
| 158 | S | 14.51 | 1.849 | | | | | | | | | | | |
| 159 | I | 10.79 | 1.272 | | | | | | | | | | | |
| 160 | S | 7.50 | 0.762 | G | | | | | | | | | | |
| 161 | Y | 8.29 | 0.886 | G | Y | | | | | | | | | |
| 162 | P | 8.39 | 0.901 | P | | | | | | | | | | |
| 163 | A | 9.26 | 1.035 | | | | | | | | | | | |
| 164 | R | 13.23 | 1.650 | | | | | | | | | | | |
| 165 | Y | 13.44 | 1.684 | | | | | | | | | | | |
| 166 | A | 18.83 | 2.519 | | | | | | | | | | | |
| 167 | N | 17.11 | 2.252 | | | | | | | | | | | |
| 168 | A | 12.96 | 1.609 | | | | | | | | | | | |
| 169 | M | 14.54 | 1.854 | | | | | | | | | | | |
| 170 | A | 11.64 | 1.404 | | | | | | | | | | | |
| 171 | V | 9.74 | 1.109 | | | | | | | | | | | |
| 172 | G | 9.49 | 1.071 | | | | | | | | | | | |
| 173 | A | 8.90 | 0.980 | A | | | | | | | | | | |
| 174 | T | 14.72 | 1.882 | | | | | | | | | | | |
| 175 | D | 13.81 | 1.741 | | | | | | | | | | | |
| 176 | Q | 16.46 | 2.151 | | | | | | | | | | | |
| 177 | N | 19.02 | 2.548 | | | | | | | | | | | |
| 178 | N | 19.77 | 2.664 | | | | | | | | | | | |
| 179 | N | 18.15 | 2.413 | | | | | | | | | | | |
| 180 | R | 14.74 | 1.884 | | | | | | | | | | | |
| 181 | A | 10.60 | 1.243 | | | | | | | | | | | |
| 182 | S | 11.70 | 1.414 | | | | | | | | | | | |
| 183 | F | 8.20 | 0.871 | F | | | | | | | | | | |
| 184 | S | 9.57 | 1.084 | | | | | | | | | | | |
| 185 | Q | 8.97 | 0.990 | S | | | | | | | | | | |
| 186 | Y | 13.55 | 1.700 | | | | | | | | | | | |
| 187 | G | 15.66 | 2.027 | | | | | | | | | | | |
| 188 | A | 18.16 | 2.414 | | | | | | | | | | | |
| 189 | G | 15.92 | 2.067 | | | | | | | | | | | |
| 190 | L | 13.68 | 1.720 | | | | | | | | | | | |
| 191 | D | 15.47 | 1.998 | | | | | | | | | | | |
| 192 | I | 15.26 | 1.965 | | | | | | | | | | | |
| 193 | V | 13.46 | 1.686 | | | | | | | | | | | |
| 194 | A | 12.78 | 1.581 | | | | | | | | | | | |
| 195 | P | 13.36 | 1.671 | | | | | | | | | | | |
| 196 | G | 8.92 | 0.983 | G | | | | | | | | | | |
| 197 | V | 10.25 | 1.189 | | | | | | | | | | | |
| 198 | N | 11.15 | 1.328 | | | | | | | | | | | |
| 199 | V | 9.53 | 1.078 | | | | | | | | | | | |
| 200 | Q | 13.84 | 1.746 | | | | | | | | | | | |
| 201 | S | 11.68 | 1.411 | | | | | | | | | | | |
| 202 | T | 15.27 | 1.967 | | | | | | | | | | | |
| 203 | Y | 13.09 | 1.629 | | | | | | | | | | | |
| 204 | P | 14.14 | 1.792 | | | | | | | | | | | |
| 205 | G | 18.52 | 2.470 | | | | | | | | | | | |
| 206 | S | 21.32 | 2.904 | | | | | | | | | | | |
| 207 | T | 17.78 | 2.356 | | | | | | | | | | | |
| 208 | Y | 16.00 | 2.080 | | | | | | | | | | | |
| 209 | A | 11.20 | 1.335 | | | | | | | | | | | |
| 210 | S | 10.13 | 1.170 | | | | | | | | | | | |
| 211 | L | 5.56 | 0.462 | H | I | L | V | | | | | | | |
| 212 | N | 5.22 | 0.409 | A | G | K | N | S | T | | | | | |
| 213 | G | 3.99 | 0.218 | A | G | | | | | | | | | |
| 214 | T | 4.71 | 0.329 | A | S | T | | | | | | | | |
| 215 | S | 4.12 | 0.239 | A | G | K | N | P | S | T | | | | |
| 216 | M | 6.72 | 0.642 | L | M | | | | | | | | | |
| 217 | A | 8.65 | 0.941 | A | T | | | | | | | | | |
| 218 | T | 7.95 | 0.832 | A | T | | | | | | | | | |
| 219 | P | 11.11 | 1.322 | | | | | | | | | | | |
| 220 | H | 13.46 | 1.687 | | | | | | | | | | | |
| 221 | V | 13.27 | 1.656 | | | | | | | | | | | |
| 222 | A | 13.63 | 1.712 | | | | | | | | | | | |
| 223 | G | 16.90 | 2.219 | | | | | | | | | | | |
| 224 | A | 18.41 | 2.454 | | | | | | | | | | | |
| 225 | A | 18.33 | 2.441 | | | | | | | | | | | |
| 226 | A | 20.39 | 2.760 | | | | | | | | | | | |
| 227 | L | 23.16 | 3.189 | | | | | | | | | | | |
| 228 | V | 23.41 | 3.228 | | | | | | | | | | | |
| 229 | K | 24.02 | 3.323 | | | | | | | | | | | |
| 230 | Q | 26.78 | 3.750 | | | | | | | | | | | |
| 231 | K | 28.47 | 4.012 | | | | | | | | | | | |
| 232 | N | 28.93 | 4.084 | | | | | | | | | | | |
| 233 | P | 30.42 | 4.315 | | | | | | | | | | | |
| 234 | S | 32.37 | 4.617 | | | | | | | | | | | |
| 235 | W | 27.42 | 3.849 | | | | | | | | | | | |
| 236 | S | 26.75 | 3.746 | | | | | | | | | | | |
| 237 | N | 21.75 | 2.971 | | | | | | | | | | | |
| 238 | V | 24.15 | 3.343 | | | | | | | | | | | |
| 239 | Q | 26.53 | 3.712 | | | | | | | | | | | |
| 240 | I | 22.19 | 3.040 | | | | | | | | | | | |
| 241 | R | 19.82 | 2.672 | | | | | | | | | | | |
| 242 | N | 24.09 | 3.334 | | | | | | | | | | | |
| 243 | H | 24.69 | 3.427 | | | | | | | | | | | |
| 244 | L | 19.49 | 2.620 | | | | | | | | | | | |
| 245 | K | 20.48 | 2.775 | | | | | | | | | | | |
| 246 | N | 25.57 | 3.564 | | | | | | | | | | | |
| 247 | T | 24.77 | 3.439 | | | | | | | | | | | |
| 248 | A | 20.35 | 2.755 | | | | | | | | | | | |
| 249 | T | 22.55 | 3.096 | | | | | | | | | | | |
| 250 | S | 24.27 | 3.362 | | | | | | | | | | | |
| 251 | L | 20.21 | 2.733 | | | | | | | | | | | |
| 252 | G | 23.36 | 3.220 | | | | | | | | | | | |
| 253 | S | 21.15 | 2.878 | | | | | | | | | | | |
| 254 | T | 21.46 | 2.926 | | | | | | | | | | | |
| 255 | N | 19.57 | 2.633 | | | | | | | | | | | |
| 256 | L | 17.37 | 2.292 | | | | | | | | | | | |
| 257 | Y | 17.33 | 2.287 | | | | | | | | | | | |
| 258 | G | 17.43 | 2.302 | | | | | | | | | | | |
| 259 | S | 20.10 | 2.715 | | | | | | | | | | | |
| 260 | G | 18.89 | 2.528 | | | | | | | | | | | |
| 261 | L | 18.35 | 2.444 | | | | | | | | | | | |
| 262 | V | 16.86 | 2.213 | | | | | | | | | | | |
| 263 | N | 22.33 | 3.062 | | | | | | | | | | | |
| 264 | A | 21.22 | 2.890 | | | | | | | | | | | |
| 265 | E | 26.13 | 3.650 | | | | | | | | | | | |
| 266 | A | 25.91 | 3.617 | | | | | | | | | | | |
| 267 | A | 23.20 | 3.196 | | | | | | | | | | | |
| 268 | T | 25.85 | 3.607 | | | | | | | | | | | |
| 269 | R | 30.12 | 4.269 | | | | | | | | | | | |

TABLE 4

| amino acid # | 61 | 118 | 119 | 120 | 122 | 151 | 203 | 210 | 219 | 220 | 288 | 291 | 292 | 315 | 321 | 342 | 345 | 348 |
|---|---|---|---|---|---|---|---|---|---|---|---|---|---|---|---|---|---|---|
| E. cloacae | L | L | Q | V | D | N | R | V | A | Y | S | A | L | T | F | S | N | R |
| A. sobria | I | L | Q | F | D | N | S | V | A | Y | P | S | L | T | F | N | I | R |
| E. coli | L | L | Q | I | D | N | R | V | A | Y | S | A | L | T | F | S | N | R |
| O. anthropi | I | L | Q | F | D | N | S | V | A | Y | S | A | L | T | F | N | I | R |
| P. aeroginosa | I | L | Q | F | D | N | G | V | G | Y | T | A | L | T | F | N | N | R |
| S. enteriditis | L | L | Q | V | D | N | K | V | S | Y | N | A | L | T | F | N | N | R |
| Y. enterolitica | L | L | Q | L | D | N | K | V | A | Y | N | A | L | T | F | N | N | R |
| IRL1.8.1 | | | | | | | | | P | | | | | | | | | |
| IRL1.8.4 | M | | | | | | | A | | | | | | | | | | |
| IRL1.8.5 | M | | | | | | | | P | | | | | | | | | |
| IRL1.8.10 | | | | | E | | | | | | | | | | | N | | P |
| IRL1.8.11 | | | | | | | | | | N | | | | | | | I | |
| IRL1.8.14 | | | | | | | | | | N | | | | | | | | |
| IRL1.8.23 | | | K | | | | | | | | | | | | | | | |
| IRL1.8.24 | | | | | | | | | | N | A | | | | | | | |
| IRL1.8.25 | | | | | | | | | | N | | | | | | | | |
| IRL1.6.1 | | | | | | | | | | | T | | | | | | | K |
| IRL2.8.1 | | | | | | | | | | | | | | | | | | |
| IRL2.8.3 | | | | | | | | | | | T | | | | | | I | |
| IRL2.8.4 | | | | F | | | | | G | | N | | | | | | | |
| IRL2.8.6 | | | | | | | | | | | | | | | | | | |
| IRL2.8.7 | | | | | | | | | | | | | | | | | | |
| IRL2.8.8 | | | | F | | | | | | | N | | | | | | I | |
| IRL2.8.9 | | | | F | | | | | | | | | | | | | | |
| IRL2.8.12 | | | | | | | | | | | | | | | | | | |
| IRL2.8.13 | | | | F | | | | | | | | | | | | | I | |
| IRL2.8.14 | | | | | | | | | | | | | | | | | | |
| IRL2.8.17 | | | | | | | | | | | | | | | | | | I |
| IRL2.8.29 | | | | | | | K | | | | | | | | | | I | |
| IRL2.3.4 | | | | | | | | | | | | | | | | | | |
| IRL2.3.5 | | | | I | | | K | | | | | | | | | | | |
| IRL2.3.6 | | | | F | | | | | | | | | | | | | | |

What is claimed is:

1. A method of creating a library of DNA sequences, said method comprising:
a) providing a DNA sequence that encodes a protein of interest;
b) providing a probability matrix for the protein;
c) providing a constraint vector for the protein;
d) applying the constraint vector to the probability matrix to produce a substitution scheme recommending substitutions at at least two residues in the protein; and
e) creating a library of DNA sequences incorporating changes in the DNA sequence that produce the recommended substitutions.

2. The method of claim 1, wherein said protein is selected from the group consisting of an esterase, dehydrogenase and hydrolase.

3. The method of claim 2, wherein said protein is selected from the group consisting of a protease, cellulase, lipase, hemicellulase, laccase, and amylase.

4. The method of claim 1, wherein said protein is selected from the group consisting of a transcription factor, growth factor, antibody, interleukin, antigen, and receptor.

5. The method of claim 1, wherein the probability matrix is based on structural characteristics selected from the group consisting of conservative residues, sequence alignments, three dimensional structure, residue environment, solvent accessibility, residue chemistry, propensity for a particular secondary structure, and combinations thereof.

6. The method of claim 1, wherein the constraint vector is based on structural characteristics known to affect protein function selected from the group consisting of proximity to the site of functionality, distance of α or β carbons, contact with residues of interest, and contact with residues that contact the residue of interest.

7. The library of claim 1, wherein said library is a phage library.

8. A method for screening a library for a protein with an increase in a property of interest, comprising:
a) providing a probability matrix for a protein of interest;
b) providing a constraint vector for the protein;
c) applying the constraint vector to the probability matrix to produce a substitution scheme recommending substitutions at at least two residues in the protein; and
d) creating a library of DNA sequences incorporating changes in the DNA sequence that produce the recommended substitutions; and
e) screening the library for a protein with an increase in the property of interest.

9. The method of claim 8, further comprising identifying a protein having an increase in the property of interest.

10. A protein produced by the method of claim 9.

11. A system for creating libraries of nucleic acid sequences that encode variants of a protein, said system comprising:
a) an initial nucleic acid sequence that encodes a desired protein;
b) a probability matrix; and
c) a constraint vector.

12. A method for improving a desired parameter of a protein of interest, comprising:
a) providing a probability matrix for the desired protein;
b) providing a constraint vector for the desired protein;
c) applying the constraint vector to the probability matrix to produce a substitution scheme recommending substitutions at at least two residues in the protein; and
d) creating a library of DNA sequences incorporating changes in the DNA sequence that produce the recommended substitutions; and
e) measuring the parameter of interest for at least two members of said library;
f) determining the sequence for at least two members of said library; and
g) using sequence comparison and correlation analysis to determine the contribution of mutations or combination of mutations on the parameter measured in step e).

13. The method of claim 12, wherein the contribution of mutations determined in step g) is used to generate a second library.

14. The method of claim 1, wherein a library comprising at least 25 unique DNA sequences is produced.

15. The method of claim 14, wherein a library comprising at least 100 unique DNA sequences is produced.

16. The method of claim 15, wherein a library comprising at least 250 unique DNA sequences is produced.

17. The method of claim 16, wherein a library comprising at least 1000 unique DNA sequences is produced.

18. The method of claim 17, wherein a library comprising at least 2500 unique DNA sequences is produced.

19. The method of claim 18, wherein a library comprising at least 10,000 unique DNA sequences is produced.

20. The method of claim 1, wherein a library of less than 10⁹ unique DNA sequences is produced.

21. The method of claim 20, wherein a library of less than 10⁶ unique DNA sequences is produced.

22. The method of claim 21, wherein a library of less than 10⁵ unique DNA sequences is produced.

23. The method of claim 1, wherein the probability matrix is an algorithm.

24. The method of claim 1, wherein the probability matrix is generated by a computer.

25. The method of claim 1, wherein the constraint vector is an algorithm.

26. The method of claim 1, wherein the constraint vector is generated by a computer.

27. The method of claim 1, wherein the constraint vector is applied to the probability matrix using a computer.

28. The method of claim 1, wherein the probability matrix is normalized.

29. The method of claim 1, wherein the DNA sequence is generated from DNA shuffling.

30. The method of claim 9, further comprising using a DNA sequence encoding the protein having an increase in the property of interest in a DNA shuffling process.

31. A method of creating a library of DNA sequences, said method comprising:
a) providing a substitution scheme produced by applying a constraint vector to a probability matrix wherein the substitution scheme recommends substitutions at at least two residues in a protein of interest; and
b) creating a library of DNA sequences incorporating substitutions in a DNA sequence encoding the protein of interest to create a library comprising the recommended substitutions.
